SEQUENCE LISTING (35 sequences)

SEQ ID NO: 1 - protein, 184 aa, synthetic construct
  REGION 1..184: note = recombinant sequence
MSRTAYTVGALLLLLGTLLPAAEGKKKGSQGAIPPPDKAQHNDSEQTQSPQQPGSRNRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 2 - protein, 139 aa, synthetic construct
  REGION 1..139: note = recombinant sequence
MGSSHHHHHHSSGENLYFQGSAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 3 - protein, 10 aa, synthetic construct
  REGION 1..10: note = recombinant sequence
GYTFTDYYMH

SEQ ID NO: 4 - protein, 5 aa, synthetic construct
  REGION 1..5: note = recombinant sequence
DYYMH

SEQ ID NO: 5 - protein, 17 aa, synthetic construct
  REGION 1..17: note = recombinant sequence
LVDPEDGETIYAEKFQG

SEQ ID NO: 6 - protein, 15 aa, synthetic construct
  REGION 1..15: note = recombinant sequence
DARGSGSYYPNHFDY

SEQ ID NO: 7 - protein, 17 aa, synthetic construct
  REGION 1..17: note = recombinant sequence
KSSQSVLYSSNNKNYLA

SEQ ID NO: 8 - protein, 7 aa, synthetic construct
  REGION 1..7: note = recombinant sequence
WASTRES

SEQ ID NO: 9 - protein, 8 aa, synthetic construct
  REGION 1..8: note = recombinant sequence
QQYYDTPT

SEQ ID NO: 10 - protein, 124 aa, synthetic construct
  REGION 1..124: note = recombinant sequence
QVQLVESGAEVKKPGATVKISCKVSGYTFTDYYMHWVQQAPGKGLEWMGLVDPEDGETIYAEKFQGRVTITADTSTDTAYMELSSLRSEDTAVYYCATDARGSGSYYPNHFDYWGQGTLVTVSS

SEQ ID NO: 11 - protein, 112 aa, synthetic construct
  REGION 1..112: note = recombinant sequence
DIVMTQSPDSLAVSLGERATINCKSSQSVLYSSNNKNYLAWYQQKPGQPPKLLIYWASTRESGVPDRFSGSGSGTDFTLTINSLQAEDVAVYFCQQYYDTPTFGQGTRLEIK

SEQ ID NO: 12 - protein, 124 aa, synthetic construct
  REGION 1..124: note = recombinant sequence
QVQLVQSGAEVKKPGATVKISCKVSGYTFTDYYMHWVQQAPGKGLEWMGLVDPEDGETIYAEKFQGRVTITADTSTDTAYMELSSLRSEDTAVYYCATDARGSGSYYPNHFDYWGQGTLVTVSS

SEQ ID NO: 13 - protein, 112 aa, synthetic construct
  REGION 1..112: note = recombinant sequence
DIVMTQTPDSLAVSLGERATINCKSSQSVLYSSNNKNYLAWYQQKPGQPPKLLIYWASTRESGVPDRFSGSGSGTDFTLTINSLQAEDVAVYFCQQYYDTPTFGQGTRLEIK

SEQ ID NO: 14 - protein, 448 aa, synthetic construct
  REGION 1..448: note = recombinant sequence
QVQLVESGAEVKKPGATVKISCKVSGYTFTDYYMHWVQQAPGKGLEWMGLVDPEDGETIYAEKFQGRVTITADTSTDTAYMELSSLRSEDTAVYYCATDARGSGSYYPNHFDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 15 - protein, 219 aa, synthetic construct
  REGION 1..219: note = recombinant sequence
DIVMTQSPDSLAVSLGERATINCKSSQSVLYSSNNKNYLAWYQQKPGQPPKLLIYWASTRESGVPDRFSGSGSGTDFTLTINSLQAEDVAVYFCQQYYDTPTFGQGTRLEIKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 16 - protein, 454 aa, synthetic construct
  REGION 1..454: note = recombinant sequence
QVQLVQSGAEVKKPGATVKISCKVSGYTFTDYYMHWVQQAPGKGLEWMGLVDPEDGETIYAEKFQGRVTITADTSTDTAYMELSSLRSEDTAVYYCATDARGSGSYYPNHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 219 aa, synthetic construct
  REGION 1..219: note = recombinant sequence
DIVMTQTPDSLAVSLGERATINCKSSQSVLYSSNNKNYLAWYQQKPGQPPKLLIYWASTRESGVPDRFSGSGSGTDFTLTINSLQAEDVAVYFCQQYYDTPTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 227 aa, synthetic construct
  REGION 1..227: note = recombinant sequence
QVQLVESGAEVKKPGATVKISCKVSGYTFTDYYMHWVQQAPGKGLEWMGLVDPEDGETIYAEKFQGRVTITADTSTDTAYMELSSLRSEDTAVYYCATDARGSGSYYPNHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 19 - protein, 219 aa, synthetic construct
  REGION 1..219: note = recombinant sequence
DIVMTQSPDSLAVSLGERATINCKSSQSVLYSSNNKNYLAWYQQKPGQPPKLLIYWASTRESGVPDRFSGSGSGTDFTLTINSLQAEDVAVYFCQQYYDTPTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 118 aa, synthetic construct
  REGION 1..118: note = recombinant sequence
AMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 21 - protein, 160 aa, synthetic construct
  REGION 1..160: note = recombinant sequence
KKKGSQGAIPPPDKAQHNDSEQTQSPQQPGSRNRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 22 - protein, 451 aa, synthetic construct
  REGION 1..451: note = recombinant sequence
QVQLVESGAEVKKPGATVKISCKVSGYTFTDYYMHWVQQAPGKGLEWMGLVDPEDGETIYAEKFQGRVTITADTSTDTAYMELSSLRSEDTAVYYCATDARGSGSYYPNHFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 219 aa, synthetic construct; identical to SEQ ID NO: 19
  REGION 1..219: note = recombinant sequence

SEQ ID NO: 24 - DNA, 1362 bp, synthetic construct
  misc_feature 1..1362: note = recombinant sequence
  source 1..1362: mol_type = other DNA
caagtgcaactggtggaatccggggccgaagtgaaaaagcccggagccactgtgaagatctcttgcaaagtgtccggctacaccttcaccgactattacatgcactgggtccagcaggcacctgggaagggccttgagtggatgggtctggtcgatcccgaggacggcgaaactatctacgccgagaagttccagggtcgcgtcaccatcaccgccgacacttccaccgacaccgcgtacatggagctgtccagcttgaggtccgaggacacagccgtgtactactgcgccacggatgctcggggaagcggcagctactacccgaaccacttcgactactggggacagggcactctcgtgactgtctcgagcgcttctacaaagggcccctccgtgttcccgctcgctccatcatcgaagtctaccagcggaggcactgcggctctcggttgcctcgtgaaggactacttcccggagccggtgaccgtgtcgtggaacagcggagccctgaccagcggggtgcacacctttccggccgtcttgcagtcaagcggcctttactccctgtcatcagtggtgactgtcccgtccagctcattgggaacccaaacctacatctgcaatgtgaatcacaaacctagcaacaccaaggttgacaagaaagtcgagcccaaatcgtgtgacaagactcacacttgtccgccgtgcccggcacccgaactgctgggaggtcccagcgtctttctgttccctccaaagccgaaagacacgctgatgatctcccgcaccccggaggtcacttgcgtggtcgtggacgtgtcacatgaggacccagaggtgaagttcaattggtacgtggatggcgtcgaagtccacaatgccaaaactaagcccagagaagaacagtacaattcgacctaccgcgtcgtgtccgtgctcacggtgttgcatcaggattggctgaacgggaaggaatacaagtgcaaagtgtccaacaaggcgctgccggcaccgatcgagaaaactatctccaaagcgaagggacagcctagggaacctcaagtctacacgctgccaccatcacgggatgaactgactaagaatcaagtctcactgacttgtctggtgaaggggttttaccctagcgacattgccgtggagtgggaatccaacggccagccagagaacaactacaagactacccctccagtgctcgactcggatggatcgttcttcctttactcgaagctcaccgtggataagtcccggtggcagcagggaaacgtgttctcctgctcggtgatgcatgaagccctccataaccactatacccaaaagtcgctgtccctgtcgccgggaaag

SEQ ID NO: 25 - DNA, 657 bp, synthetic construct
  misc_feature 1..657: note = recombinant sequence
  source 1..657: mol_type = other DNA
gacattgtgatgacccagtcccccgattcgcttgcggtgtccctgggagaacgggccaccattaactgcaagagctcacagtccgtcctgtattcatcgaacaacaagaattacctcgcatggtatcagcagaagcctggacagcctcccaagctgctcatctactgggctagcacccgcgaatccggggtgccggatagattctccggatcgggttcgggcactgacttcactctgactatcaactcactgcaagccgaggatgtcgcggtgtacttctgtcagcagtactacgacaccccgacctttggacaaggcaccagactggagattaagcgtacggtggccgctccctccgtgttcatcttcccaccctccgacgagcagctgaagtccggcaccgcctccgtcgtgtgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgc

SEQ ID NO: 26 - DNA, 1353 bp, synthetic construct
  misc_feature 1..1353: note = recombinant sequence
  source 1..1353: mol_type = other DNA
caagtgcaactggtggaatccggggccgaagtgaaaaagcccggagccactgtgaagatctcttgcaaagtgtccggctacaccttcaccgactattacatgcactgggtccagcaggcacctgggaagggccttgagtggatgggtctggtcgatcccgaggacggcgaaactatctacgccgagaagttccagggtcgcgtcaccatcaccgccgacacttccaccgacaccgcgtacatggagctgtccagcttgaggtccgaggacacagccgtgtactactgcgccacggatgctcggggaagcggcagctactacccgaaccacttcgactactggggacagggcactctcgtgactgtctcgagcgcttctacaaagggcccctccgtgttccctctggccccttgctcccggtccacctccgagtctaccgccgctctgggctgcctggtcaaggactacttccccgagcccgtgacagtgtcctggaactctggcgccctgacctccggcgtgcacaccttccctgccgtgctgcagtcctccggcctgtactccctgtcctccgtcgtgaccgtgccctcctccagcctgggcaccaagacctacacctgtaacgtggaccacaagccctccaacaccaaggtggacaagcgggtggaatctaagtacggccctccctgccccccctgccctgcccctgaatttctgggcggaccttccgtgttcctgttccccccaaagcccaaggacaccctgatgatctcccggacccccgaagtgacctgcgtggtggtggacgtgtcccaggaagatcccgaggtccagttcaattggtacgtggacggcgtggaagtgcacaatgccaagaccaagcccagagaggaacagttcaactccacctaccgggtggtgtccgtgctgaccgtgctgcaccaggactggctgaacggcaaagagtacaagtgcaaggtgtccaacaagggcctgccctccagcatcgaaaagaccatctccaaggccaagggccagccccgcgagccccaggtgtacaccctgccccctagccaggaagagatgaccaagaaccaggtgtccctgacctgtctggtcaagggcttctacccctccgacattgccgtggaatgggagtccaacggccagcccgagaacaactacaagaccaccccccctgtgctggacagcgacggctccttcttcctgtactctcggctgaccgtggacaagtcccggtggcaggaaggcaacgtcttctcctgctccgtgatgcacgaggccctgcacaaccactacacccagaagtccctgtccctgagcctgggcaag

SEQ ID NO: 27 - DNA, 657 bp, synthetic construct; identical to SEQ ID NO: 25
  misc_feature 1..657: note = recombinant sequence
  source 1..657: mol_type = other DNA

SEQ ID NO: 28 - protein, 448 aa, synthetic construct
  REGION 1..448: note = recombinant sequence
QVQLVQSGAEVKKPGATVKISCKVSGYTFTDYYMHWVQQAPGKGLEWMGLVDPEDGETIYAEKFQGRVTITADTSTDTAYMELSSLRSEDTAVYYCATDARGSGSYYPNHFDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 29 - protein, 219 aa, synthetic construct
  REGION 1..219: note = recombinant sequence
DIVMTQTPDSLAVSLGERATINCKSSQSVLYSSNNKNYLAWYQQKPGQPPKLLIYWASTRESGVPDRFSGSGSGTDFTLTINSLQAEDVAVYFCQQYYDTPTFGQGTRLEIKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 30 - protein, 454 aa, synthetic construct
  REGION 1..454: note = recombinant sequence
QVQLVESGAEVKKPGATVKISCKVSGYTFTDYYMHWVQQAPGKGLEWMGLVDPEDGETIYAEKFQGRVTITADTSTDTAYMELSSLRSEDTAVYYCATDARGSGSYYPNHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 219 aa, synthetic construct; identical to SEQ ID NO: 19
  REGION 1..219: note = recombinant sequence

SEQ ID NO: 32 - protein, 227 aa, synthetic construct
  REGION 1..227: note = recombinant sequence
QVQLVQSGAEVKKPGATVKISCKVSGYTFTDYYMHWVQQAPGKGLEWMGLVDPEDGETIYAEKFQGRVTITADTSTDTAYMELSSLRSEDTAVYYCATDARGSGSYYPNHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 33 - protein, 219 aa, synthetic construct; identical to SEQ ID NO: 17
  REGION 1..219: note = recombinant sequence

SEQ ID NO: 34 - protein, 451 aa, synthetic construct
  REGION 1..451: note = recombinant sequence
QVQLVQSGAEVKKPGATVKISCKVSGYTFTDYYMHWVQQAPGKGLEWMGLVDPEDGETIYAEKFQGRVTITADTSTDTAYMELSSLRSEDTAVYYCATDARGSGSYYPNHFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 219 aa, synthetic construct; identical to SEQ ID NO: 17
  REGION 1..219: note = recombinant sequence

(19) United States
(12) Patent Application Publication
(10) Pub. No.: US 2024/0076365 A1
(43) Pub. Date: Mar. 7, 2024

(54) GREMLIN-1 CRYSTAL STRUCTURE AND INHIBITORY ANTIBODY

(71) Applicant: UCB BIOPHARMA SRL, BRUSSELS (BE)

(72) Inventors: NEESHA DEDI, SLOUGH (GB); BREDA TWOMEY, SLOUGH (GB); MICHAEL JOHN WRIGHT, SLOUGH (GB); GARETH CHARLES GLYNDWR DAVIES, SLOUGH (GB); DAVID JAMES MCMILLAN, SLOUGH (GB)

(21) Appl. No.: 18/502,188

(22) Filed: Nov. 6, 2023

Related U.S. Application Data
(62) Division of application No. 17/172,109, filed on Feb. 10, 2021, now Pat. No. 11,807,680.
Application No. 17/172,109 is a division of application No. 16/470,996, filed on Jun. 19, 2019, now Pat. No. 10,947,304.

(30) Foreign Application Priority Data
Dec. 19, 2016 (GB) 1621635.0

(51) Int. Cl. C07K 16/22; A61P 9/12; C07K 14/51; G01N 33/68

(52) U.S. Cl. CPC C07K 16/22; A61P 9/12; C07K 14/51; G01N 33/6854; A61K 2039/505

(57) ABSTRACT

This invention relates to crystals of the human Gremlin-1 protein, and the human Gremlin-1 protein in complex with an inhibitory antibody. The invention also relates to the structure of human Gremlin-1 (on its own, or in complex with the antibody) and uses of these structures in screening for agents which modulate Gremlin-1 activity. The invention further provides antibodies which bind an allosteric inhibitory site on Gremlin-1, together with pharmaceutical compositions and medical uses of such antibodies and agents identified by the screening methods.

### CROSS-REFERENCE TO RELATED APPLICATIONS

This application is a divisional of U.S. application Ser. No. 17/172,109, filed Feb. 10, 2021, now U.S. Pat. No. 11,807,680, which is a divisional of U.S. application Ser. No. 16/470,996, filed Jun. 19, 2019, now U.S. Pat. No. 10,947,304, which is the U.S. national stage application of International Patent Application No. PCT/EP2017/083650, filed Dec. 19, 2017.

The Sequence Listing for this application is labeled “Seq-List.xml” which was created on Oct. 19, 2023 and is 54,527 bytes. The entire content of the sequence listing is incorporated herein by reference in its entirety.

### FIELD OF THE INVENTION

This invention relates to crystals of the human Gremlin-1 protein, and the human Gremlin-1 protein in complex with an inhibitory antibody. The invention also relates to the structure of human Gremlin-1 (on its own, or in complex with the antibody) and uses of these structures in screening for agents which modulate Gremlin-1 activity. The invention further provides antibodies which bind an allosteric inhibitory site on Gremlin-1, together with pharmaceutical compositions and medical uses of such antibodies and agents identified by the screening methods.

### BACKGROUND OF THE INVENTION

Gremlin-1 (also known as Drm and CKTSF1B1) is a 184 amino acid glycoprotein which forms part of the DAN family of cystine-knot secreted proteins (along with Cerberus and Dan amongst others). Gremlin binds and inhibits the ability of BMP-2, 4, and 7 to signal along with a documented pro-angiogenic role possibly through agonism of VEGFR2. The main role of Gremlin-1 is during development, in which it is vital during kidney formation and during limb bud formation. These vital roles make gremlin homozygous knock-outs lethal in embryonic mice.

In adulthood, increased levels of gremlin have been associated with idiopathic pulmonary fibrosis and pulmonary arterial hypertension in which BMP-2, 4 and 7 signalling is reduced with an associated rise in TGF-β levels. In both diabetic and chronic allograft nephropathy, Gremlin-1 expression has been correlated with fibrosis score.

Increased levels of gremlin are also linked to scleroderma, diabetic nephropathy and colorectal cancer. Gremlin-1 has been shown to activate cancer cell invasion and proliferation and is thought to play a role in uterine cervix, lung, ovary, kidney, breast, colon, pancreatic and sarcoma carcinomas.

To date, there have been a number of challenges associated with studying Gremlin-1 and there is a lack of general understanding around Gremlin-1 (and its partner Gremlin-2). BMP biology is complex, and high homology exists between species. Gremlin-1 is a difficult protein to work with, and there is a lack of suitable tools and reagents for studying its biology. Making Gremlin-1 is also not a straightforward process; cysteine-knot proteins are notoriously difficult to produce and the free cysteine of Gremlin-1 adds to the challenge. Gremlin-1 is difficult to express let alone purify. Until now, structural information has not been available and there is very little information on this protein in the literature.

### SUMMARY OF THE INVENTION

The term Gremlin-1 as used in the present invention typically has the sequence as set out in the UniProt entry 060565 (SEQ ID NO: 1). The term Gremlin-1 may also refer to a Gremlin-1 polypeptide which:
(a) comprises or consists of the amino acid sequence of SEQ ID NO: 1 with or without the N-terminal signal peptide, i.e. may comprise or consist of the mature peptide sequence as shown in SEQ ID NO: 21; or
(b) is a derivative having one or more amino acid substitutions, modifications, deletions or insertions relative to the amino acid sequence of SEQ ID NO: 1 with or without the N-terminal signal peptide (as shown in SEQ ID NO: 21), which retains the activity of Gremlin-1, such as the amino acid sequence of SEQ ID NO: 20.
(c) a variant thereof, such variants typically retain at least about 60%, 70%, 80%, 90%, 91%, 92%, 93%, 94% or 95% identity to SEQ ID NO: 1 (or SEQ ID NO: 20 or 21) (or even about 96%, 97%, 98% or 99% identity). In other words, such variants may retain about 60%-about 99% identity to SEQ ID NO: 1, suitably about 80%-about 99% identity to SEQ ID NO: 1, more suitably about 90%-about 99% identity to SEQ ID NO: 1 and most suitably about 95%-about 99% identity to SEQ ID NO: 1. Variants are described further below.

As discussed further below, residue numbers are typically quoted based on the sequence of SEQ ID NO: 1. However, residue numbering could readily be extrapolated by the skilled person to a derivative or variant sequence as discussed above. Where residue numbers are quoted, the invention also encompasses these residues on a variant or derivative sequence.

The present inventors have crystallised human Gremlin-1 alone, and in complex with an antibody termed Ab 7326 (Fab fragments). Crystallisation of Gremlin-1 has allowed putative residues in the BMP binding site to be determined. Furthermore, crystallisation with Ab 7326, which is an allosteric inhibitory antibody, has allowed residues in the antibody epitope to be determined. Antibodies binding this epitope have potential as therapeutic agents in the treatment of diseases associated with Gremlin-1.

Accordingly, the present invention provides a crystal of Gremlin-1.

The present invention also provides the structure of human Gremlin-1 as defined by the coordinates in Table 1.

Furthermore, the invention provides:
A machine readable data storage medium which comprises data storage material encoded with machine readable data defined by the structure coordinates of Gremlin-1 in Table 1 or coordinates defining homologues of the structure.
Use of the structure of Gremlin-1 as defined by the coordinates in Table 1 as a structural model.
Agents identified by use of the structural model.
A method of screening for modulatory agents of Gremlin-1 activity, comprising the steps of:
   (a) identifying a ligand binding site from the structural coordinates in Table 1; (b) identifying candidate agents which interact with at least part of the ligand binding site; and
   (c) obtaining or synthesising said agent.
   A Gremlin-1 modulating agent as identified by the screening method.
An antibody which binds to an epitope on Gremlin-1 comprising at least one residue selected from Ile131, Lys147, Lys148, Phe149, Thr150, Thr151, Arg169, Lys174 and Gln175, wherein the residue numbering is based on SEQ ID NO: 1.
An anti-Gremlin-1 antibody which comprises heavy chain complementarity determining region (HCDR) sequences contained within a heavy chain variable region (HCVR) of SEQ ID NO: 10 or 12 and/or light chain complementarity determining region (LCDR) sequences contained within a light chain variable region (LCVR) of SEQ ID NO: 11 or 13.
An anti-Gremlin-1 antibody which comprises at least one HCDR sequence selected from SEQ ID NOs: 3, 4, 5 and 6 and/or at least one LCDR sequence selected from SEQ ID NOs: 7, 8 and 9.
An isolated polynucleotide encoding the antibodies.
An expression vector carrying the polynucleotide.
A host cell comprising the vector.
A method of producing the antibody, comprising culturing the host cell under conditions permitting production of the antibody, and recovering the produced antibody.
A pharmaceutical composition comprising an antibody.
An antibody or pharmaceutical composition for use in a method of treatment of the human or animal body by therapy.
A method of treating or preventing renal fibrosis such as diabetic nephropathy, idiopathic pulmonary fibrosis, pulmonary arterial hypertension, angiogenesis and/or cancer comprising administering a therapeutically effective amount of an antibody or a pharmaceutical composition to a patient in need therefore.

### BRIEF DESCRIPTION OF THE FIGURES

FIG. 1 (Table 1) presents the structural data for Gremlin-1 crystallography.

FIG. 2 shows the structure of human Gremlin-1. Ribbon representations of each monomer are shown in different shades of grey, fingers 1 & 2 (F1 & F2) are marked along with the “wrist” regions (w) and the cystine-knots (CK). Cysteines forming disulphide bonds are shown as black sticks.

FIG. 3 shows a sequence alignment of human Gremlin-1 and mouse Gremlin-2 (PRDC). Residues marked with an asterisk are important in BMP binding and residues forming key contacts in the dimer interface are boxed.

FIG. 4 shows surface rendering highlighting the hydrophobic BMP binding residues. The monomers are shown in two shades of grey and the six key residues involved in BMP binding are shown in white.

FIG. 5 presents an overlay of human Gremlin-1 and mouse Gremlin-2. The upper image is a ribbon representation with the two proteins aligned. The lower image shows in detail the amino acids involved in BMP binding as sticks (with mouse Gremlin-2 in white and human Gremlin-1 in black).

FIG. 6(A) shows a representation of the inhibitory effects of full length Gremlin-1 in the Hek Id1 reporter gene assay.

FIG. 6(B) shows a representation of the inhibitory effects of truncated Gremlin-1 in the Hek Id1 reporter gene assay.

FIG. 7 shows percentage restoration of signal for the immunisation derived antibodies in the Hek-Id1 reporter gene assay.

FIG. 8 shows percentage restoration of signal for library derived antibodies in the Hek-Id1 reporter gene assay.

FIG. 9 shows results for the Hek-Id1 reporter gene assay with titrations of human Gremlin (FIG. 9A) and mouse Gremlin (FIG. 9B) and the effect of antibody 7326 (shown as antibody PB376) in restoring signalling of BMP.

FIG. 10 shows a structural model of the Gremlin-Fab complex, with the possible BMP binding regions and the Fab epitope highlighted.

FIG. 11 shows surface rendering depicting each Gremlin-1 monomer in two shades of grey, the six key residues identified by mutagenesis to be involved in BMP binding in black and all residues on the surface within 6 Å of those six key residues.

FIG. 12. Assessment of right ventricular systolic pressures (RVSP). The effects of anti-Gremlin 1 antibodies on RVSPs were assessed in normoxia and hypoxia/SU5416 treated C57Bl/6 mice. The effects of anti-Gremlin 1 (n=8), IgG1 antibody control (n=6), PBS (n=2), Imatinib (n=8), on pulmonary arterial hypertension (PAH) development were determined in female C57Bl/6 mice injected sub-cutaneously every three days with SU5416 (20 mg/kg) following exposure to chronic normobaric hypoxia (10% O₂) or normoxia for 21 days. RVSP were determined and the mean RVSP±SEM plotted.

*P<0.05; **P<0.01; ***P<0.005; ****P<0.001 by one-way ANOVA.

FIG. 13. Assessment of mean arterial systemic pressures (MABP). The effects of anti-Gremlin 1 antibodies on MABP were assessed in hypoxia/SU5416 C57Bl/6 mice treated with anti-Gremlin 1 (n=4), IgG1 antibody control (n=4), Imatinib (n=4) and normoxia/SU5416 anti-Gremlin 1 (n=4), IgG1 antibody control (n=4) and the MABP SEM plotted after 21 days.

*P<0.05; **P<0.01; ***P<0.005; ****P<0.001 by one-way ANOVA.

FIG. 14. Assessment of right ventricular hypertrophy. The effects of anti-Gremlin 1 antibodies on right heart hypertrophy (RV/LV+S) were assessed in hypoxia/SU5416 C57Bl/6 mice. The effects of anti-Gremlin 1 (n=8), IgG antibody control (n=6), PBS (n=2), Imatinib (n=8) on pulmonary arterial hypertension (PAH) development were determined in female C57Bl/6 mice injected sub-cutaneously every three days with SU5416 (20 mg/kg) following exposure to chronic normobaric hypoxia (10% O₂) or normoxia for 21 days. RVSP were determined and the mean RVSP±SEM plotted.

*P<0.05; **P<0.01; ***P<0.005; ****P<0.001 by one-way ANOVA.

FIG. 15. Histological assessment of pulmonary vascular muscularisation. The effects of anti-Gremlin 1 antibodies were assessed. Paraffin embedded lung sections isolated from either anti-Gremlin 1 (n=6), IgG antibody control (n=6), Imatinib (n=6) were stained for smooth muscle actin (αSMA) to assess the extent of muscularisation and Von Willebrand factor (vWF) to identify endothelial cells by immune-histochemistry. Lung sections were digitised by Nanozoomer virtual microscopy (Hamamatsu, Welwyn Garden City, UK) and >40 vessels per group were scored by independent blinded observers as non, partially or fully muscularised. Mean scores±SEM of each group of the modal score of each vessel were plotted. Representative images of paraffin embedded lung sections stained for αSMA and vWF of normoxia IgG1; Hypoxia/SU5416 IgG1; Hypoxia/SU5416 Imatinib; Hypoxia/SU5416 anti-Gremlin 1.

*P<0.05; **P<0.01; ***P<0.005; ****P<0.001 by one-way ANOVA.

### BRIEF DESCRIPTION OF THE SEQUENCE LISTING

SEQ ID NO: 1 shows the sequence of human Gremlin-1 including the 24 amino acid N-terminal signal sequence (Uniprot ID 060565).

SEQ ID NO: 2 shows the sequence of truncated human Gremlin-1 used in crystallography including an N-terminal tag.

SEQ ID NO: 3 shows the Ab 7326 HCDR1 (Chothia).

SEQ ID NO: 4 shows the Ab 7326 HCDR1 (Kabat).

SEQ ID NO: 5 shows the Ab 7326 HCDR2 (Kabat).

SEQ ID NO: 6 shows the Ab 7326 HCDR3 (Kabat).

SEQ ID NO: 7 shows the Ab 7326 LCDR1 (Kabat).

SEQ ID NO: 8 shows the Ab 7326 LCDR2 (Kabat).

SEQ ID NO: 9 shows the Ab 7326 LCDR3 (Kabat).

SEQ ID NO: 10 shows the Ab 7326 heavy chain variable region (variant 1).

SEQ ID NO: 11 shows the Ab 7326 light chain variable region (variant 1).

SEQ ID NO: 12 shows the Ab 7326 heavy chain variable region (variant 2).

SEQ ID NO: 13 shows the Ab 7326 light chain variable region (variant 2).

SEQ ID NO: 14 shows the mouse Ab 7326 full length IgG1 heavy chain (variant 1).

SEQ ID NO: 15 shows the mouse Ab 7326 full length IgG1 light chain (variant 1).

SEQ ID NO: 16 shows the human Ab 7326 full length IgG1 heavy chain (variant 2).

SEQ ID NO: 17 shows the human Ab 7326 full length IgG1 light chain (variant 2).

SEQ ID NO: 18 shows the Ab 7326 Fab heavy chain (variant 1).

SEQ ID NO: 19 shows the Ab 7326 Fab light chain (variant 1).

SEQ ID NO: 20 shows the sequence of truncated human Gremlin-1 used in crystallography without the N-terminal tag.

SEQ ID NO: 21 shows the sequence of mature Gremlin-1 (SEQ ID NO: 1 without the signal peptide).

SEQ ID NO: 22 shows the human IgG4P heavy chain (variant 1).

SEQ ID NO: 23 shows the human IgG4P light chain (variant 1).

SEQ ID NO: 24 shows the human IgG1 heavy chain DNA (variant 1).

SEQ ID NO: 25 shows the human IgG1 light chain DNA (variant 1).

SEQ ID NO: 26 shows the human IgG4P heavy chain DNA (variant 1).

SEQ ID NO: 27 shows the human IgG4P light chain DNA (variant 1).

SEQ ID NO: 28 shows the mouse full length IgG1 heavy chain (variant 2).

SEQ ID NO: 29 shows the mouse full length IgG1 light chain (variant 2).

SEQ ID NO: 30 shows the human full length IgG1 heavy chain (variant 1).

SEQ ID NO: 31 shows the human full length IgG1 light chain (variant 1).

SEQ ID NO: 32 shows the Fab heavy chain (variant 2).

SEQ ID NO: 33 shows the Fab light chain (variant 2).

SEQ ID NO: 34 shows the human IgG4P heavy chain (variant 2).

SEQ ID NO: 35 shows the human IgG4P light chain (variant 2).

### DETAILED DESCRIPTION OF THE INVENTION

### Gremlin-1 Crystal Structure

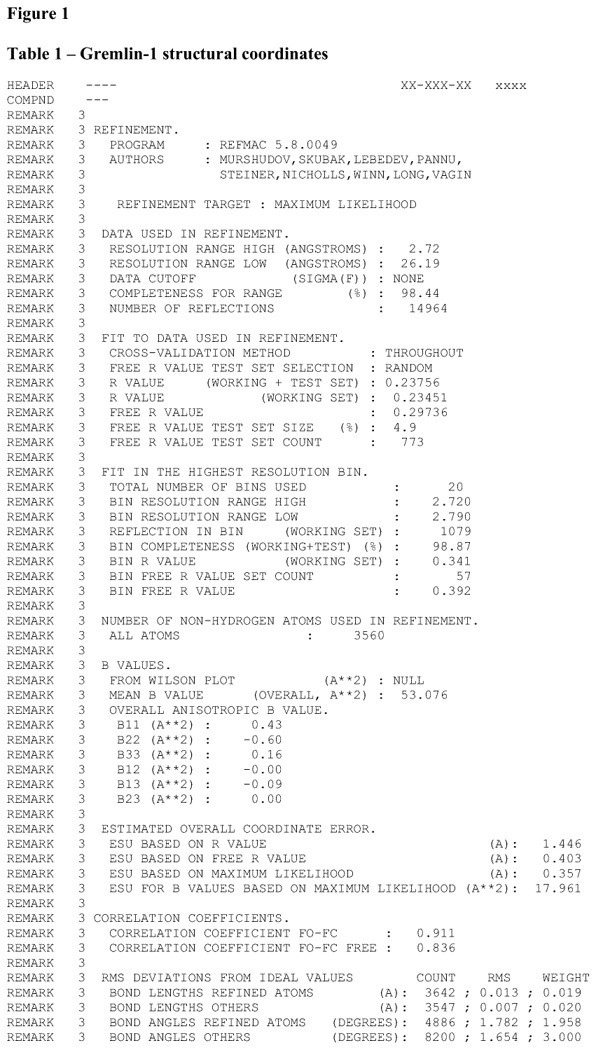

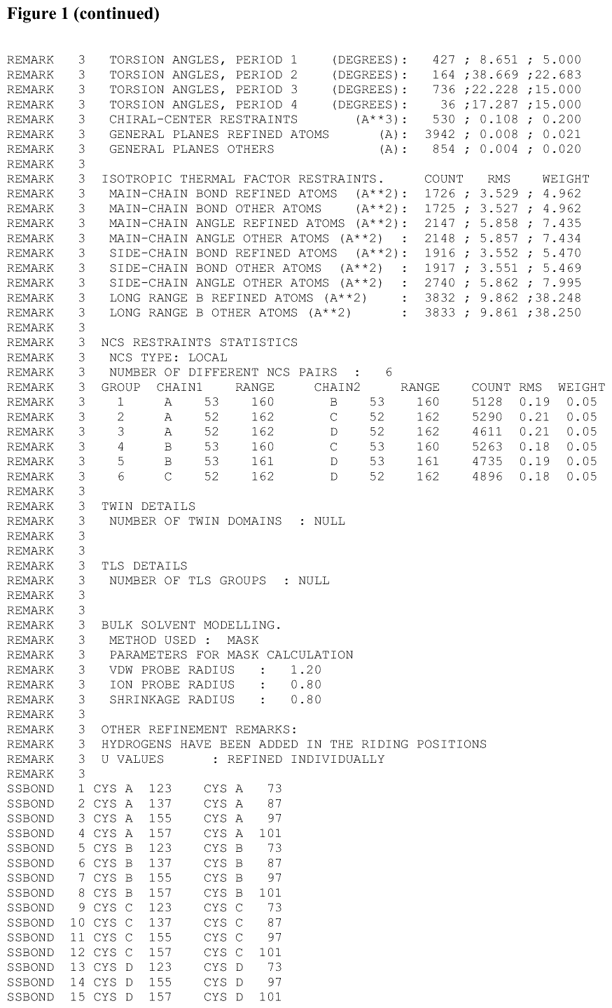

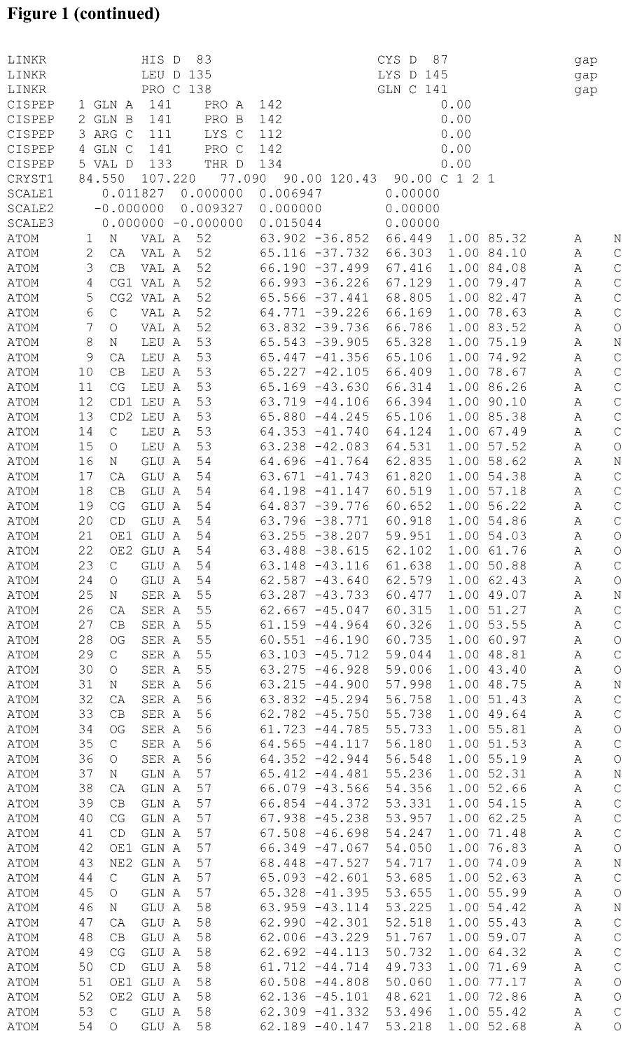

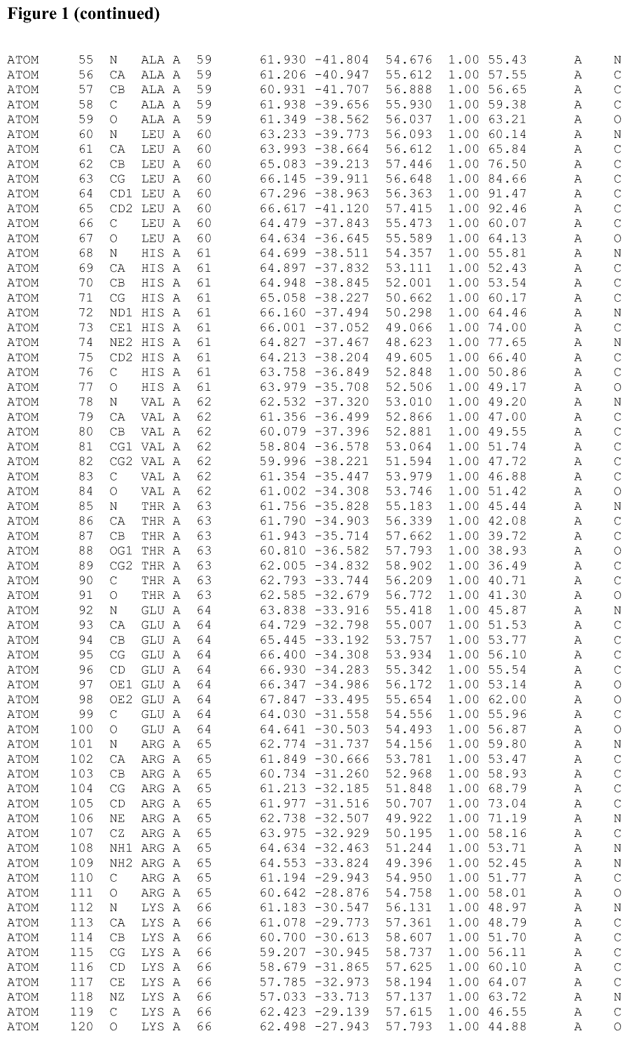

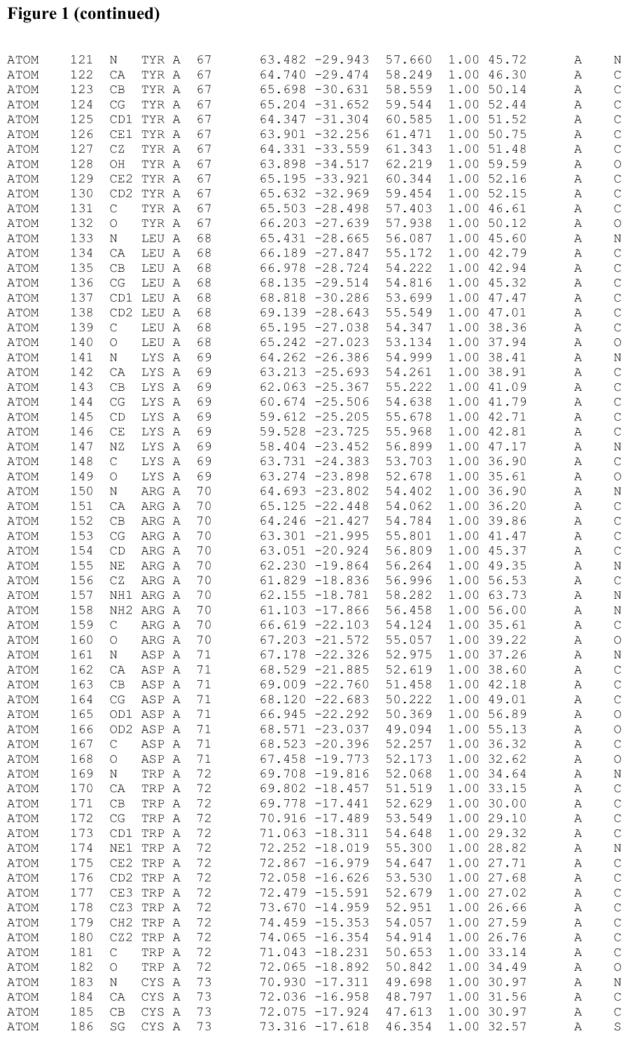

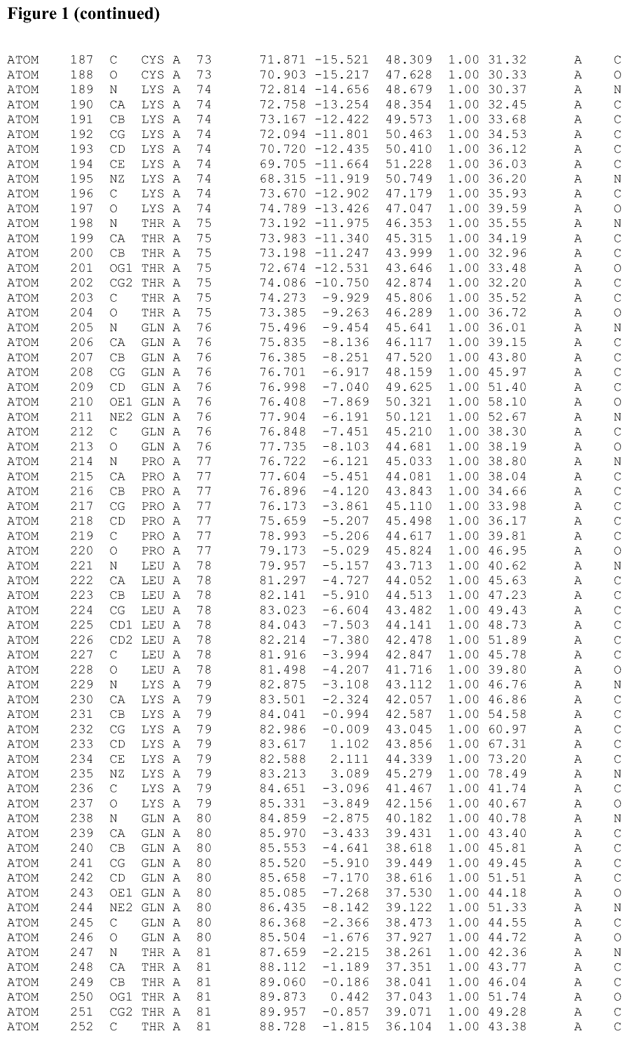

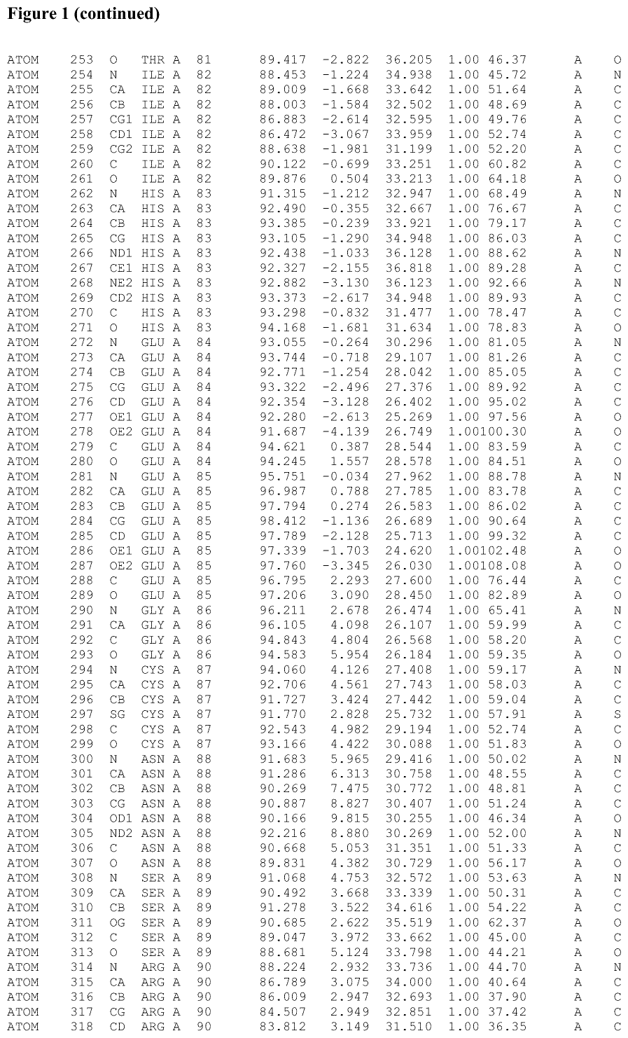

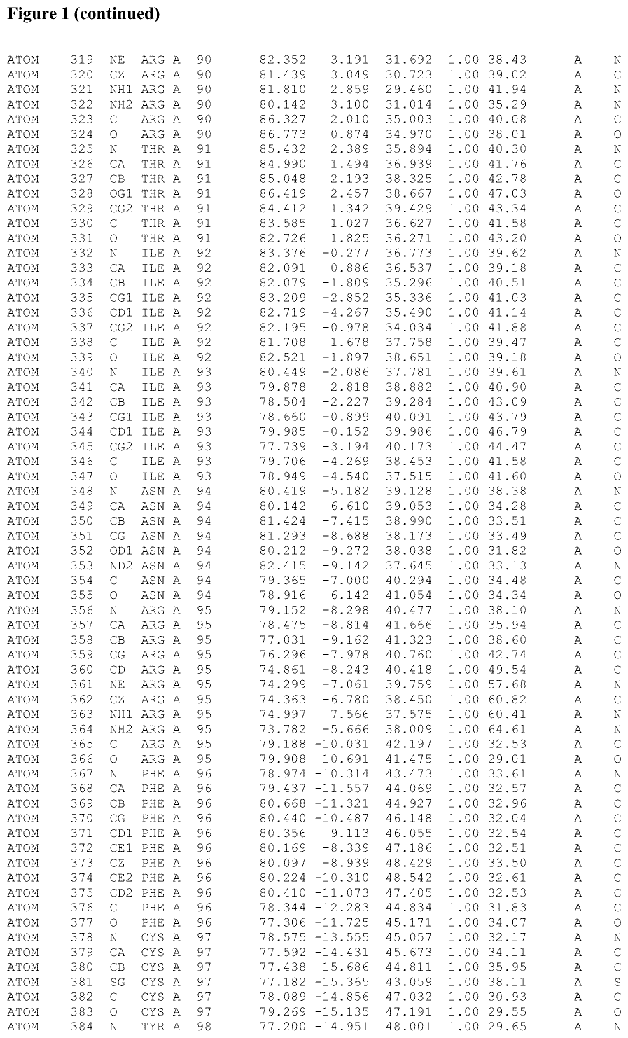

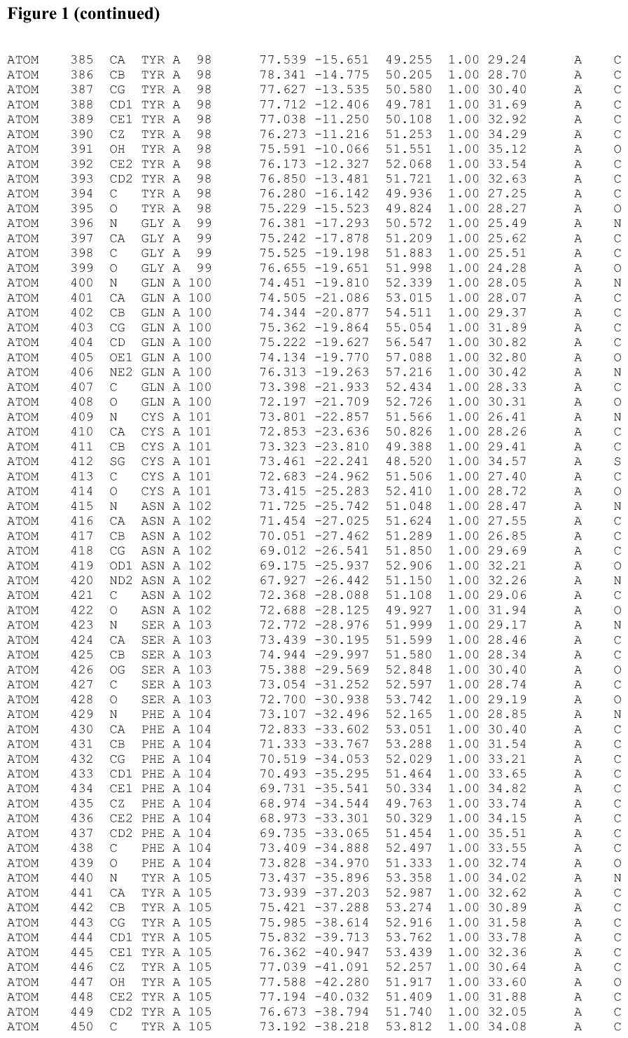

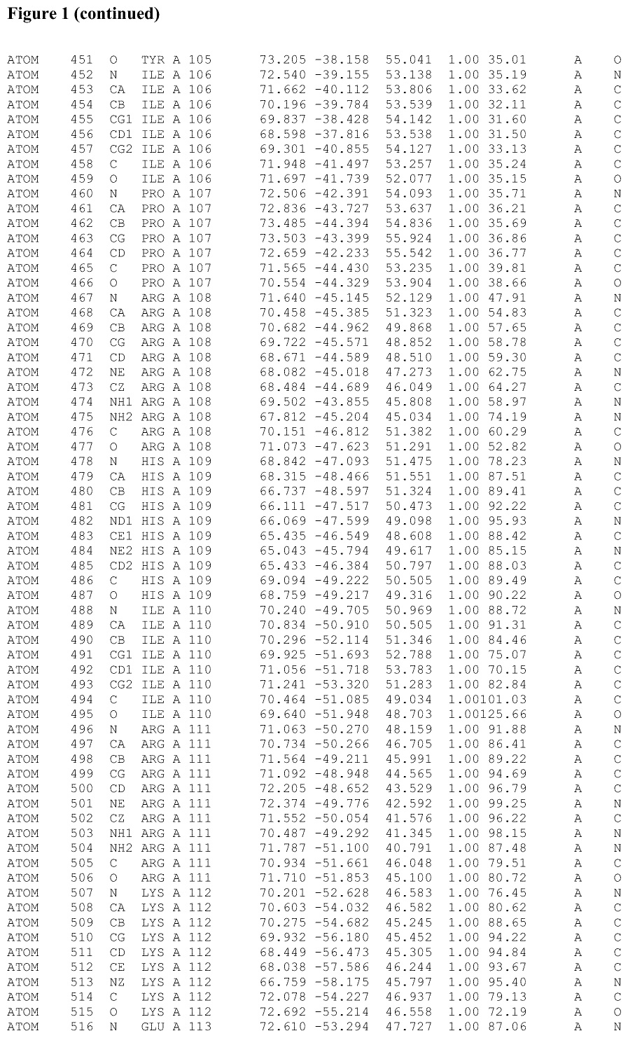

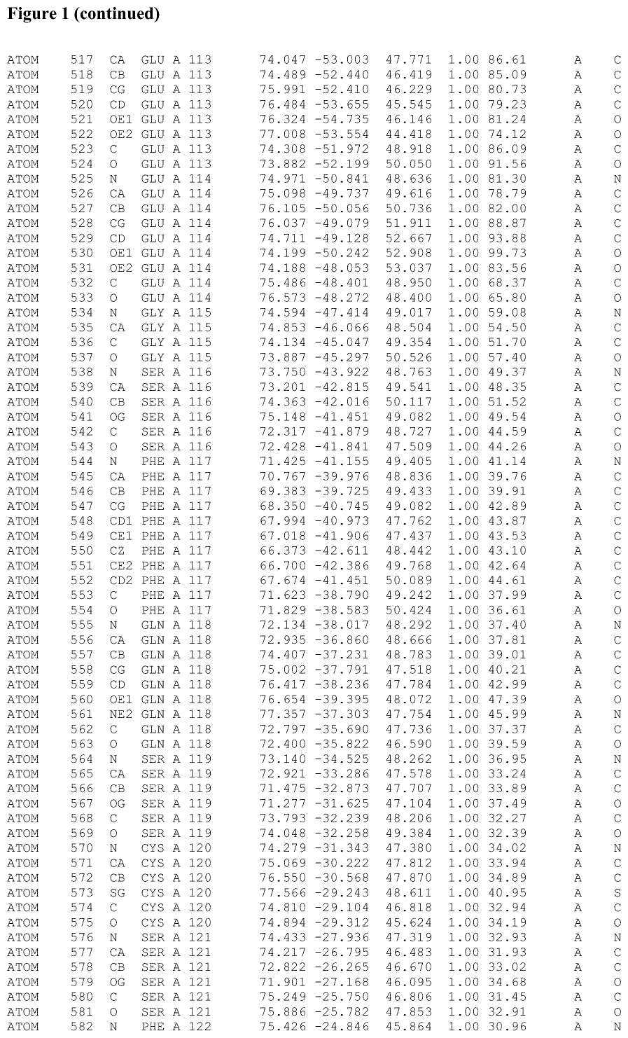

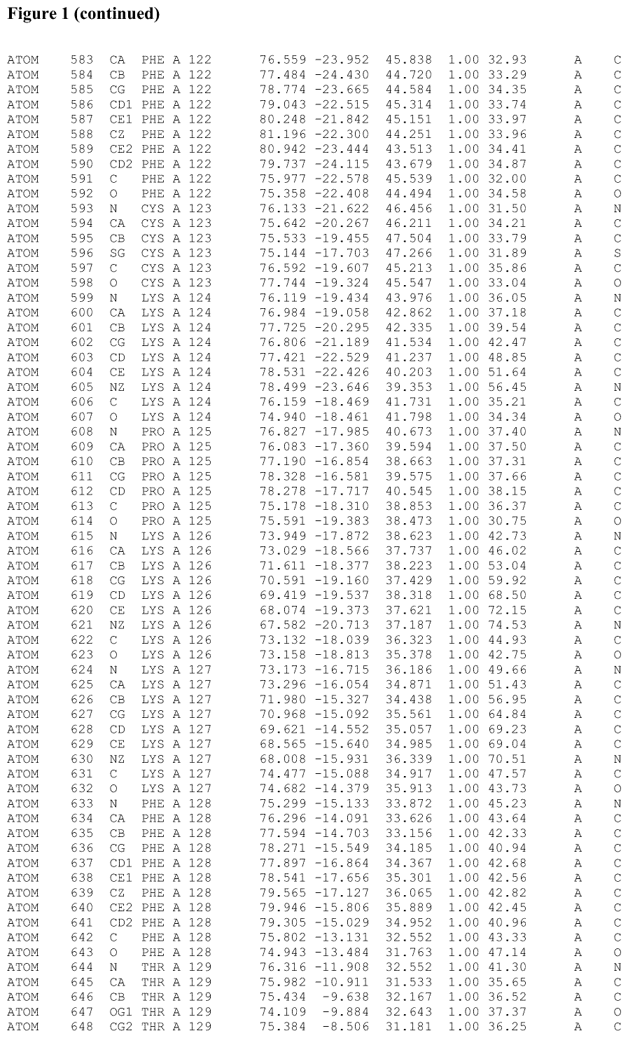

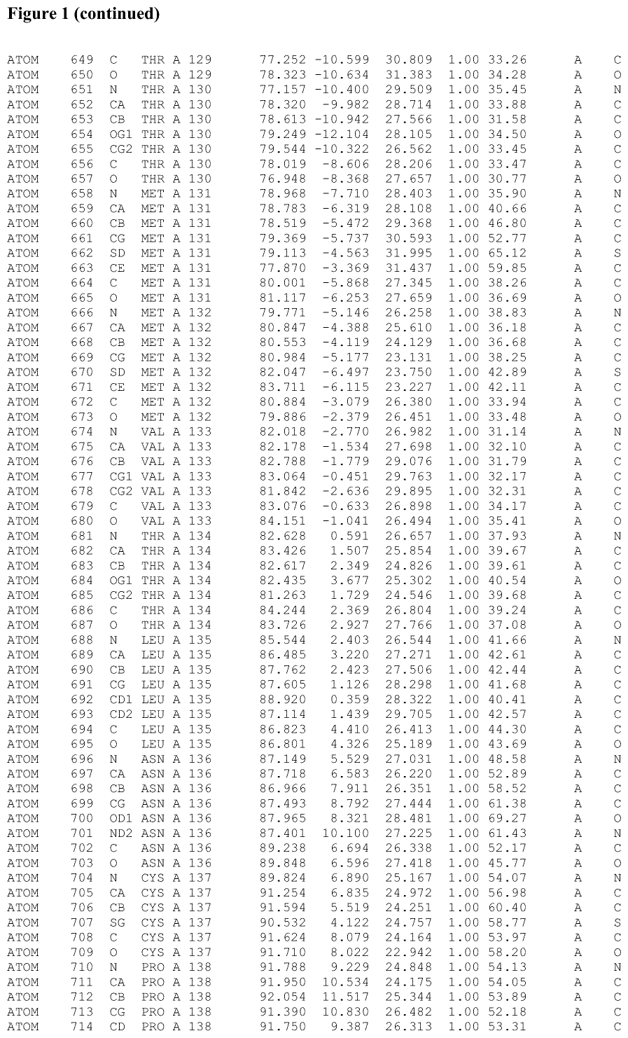

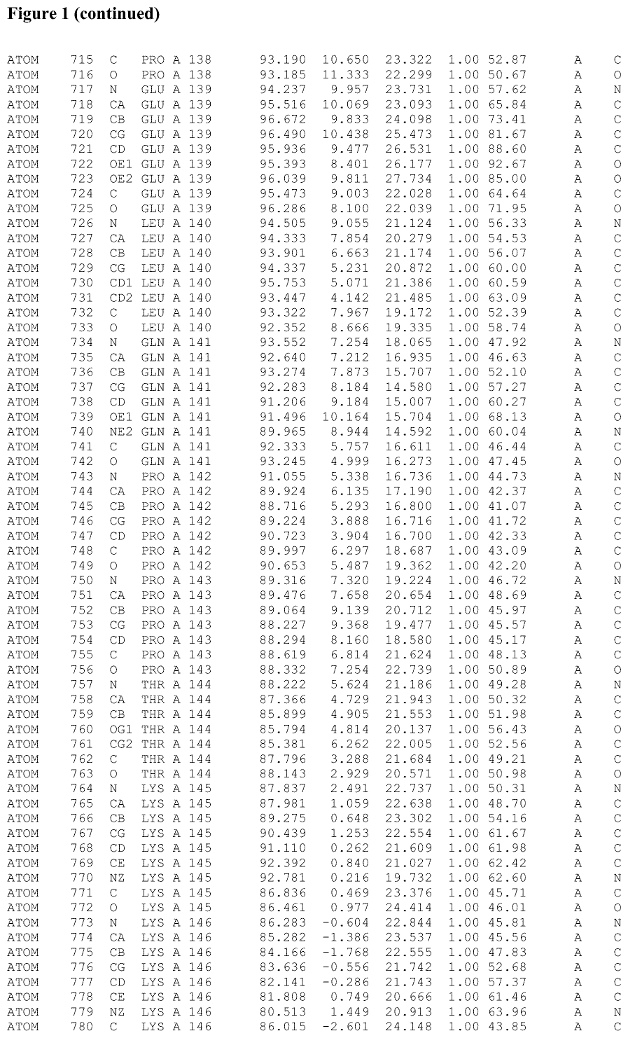

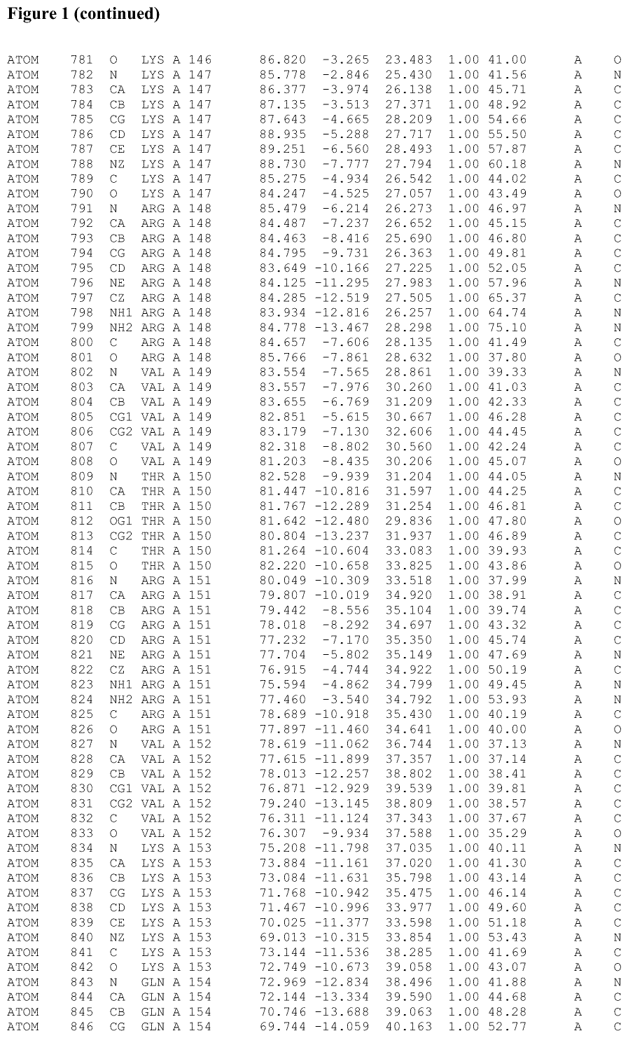

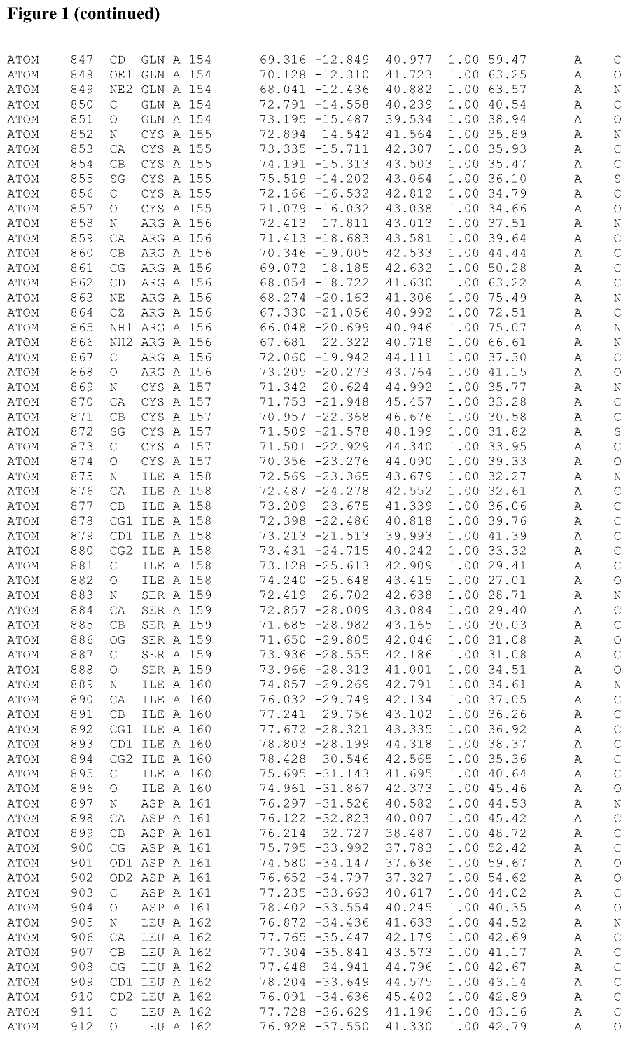

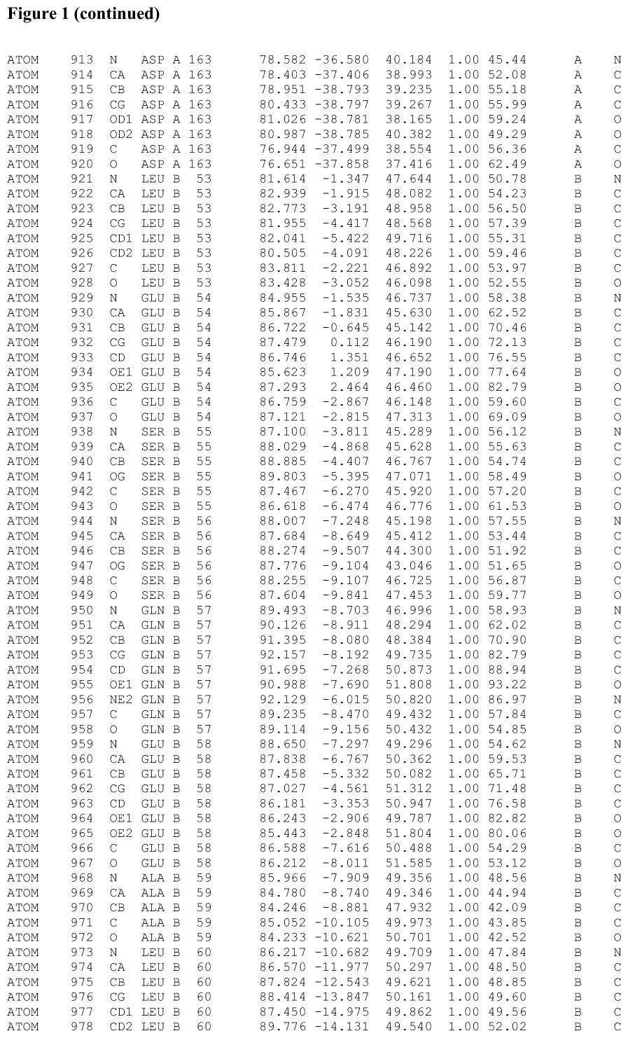

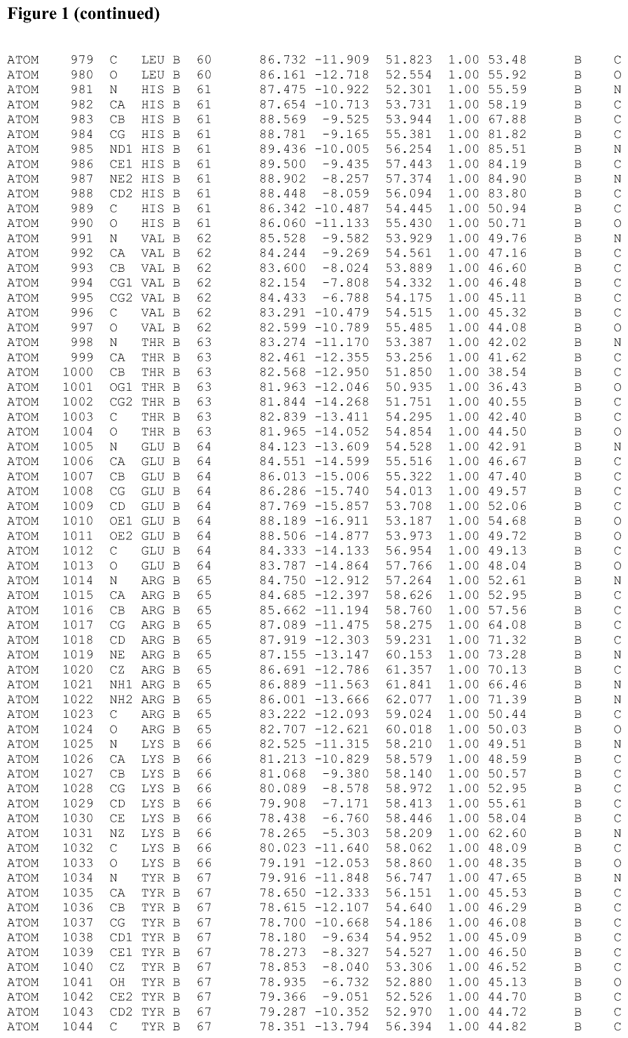

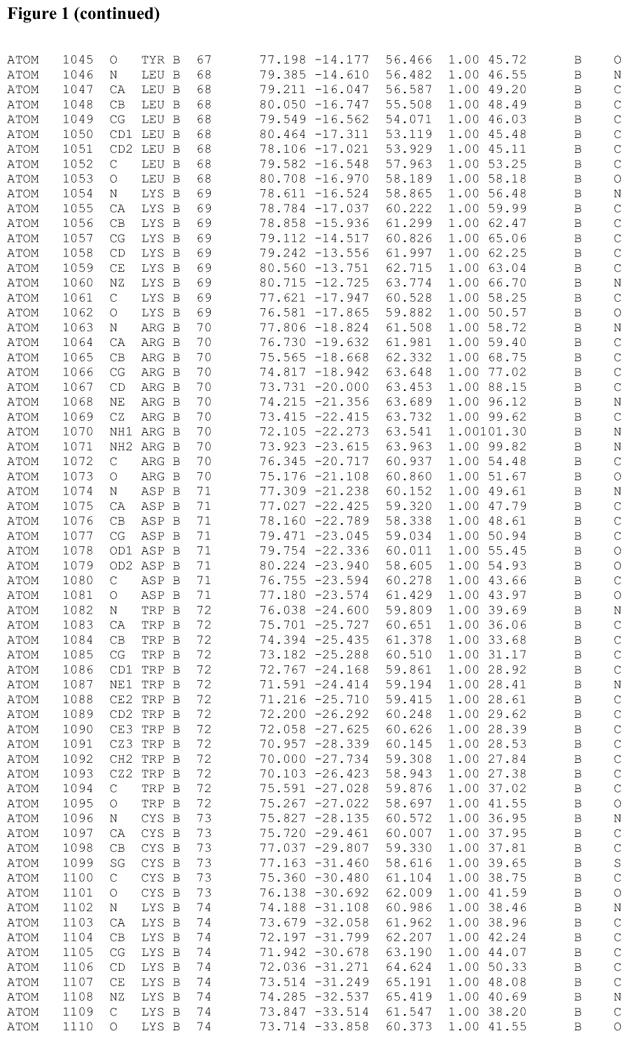

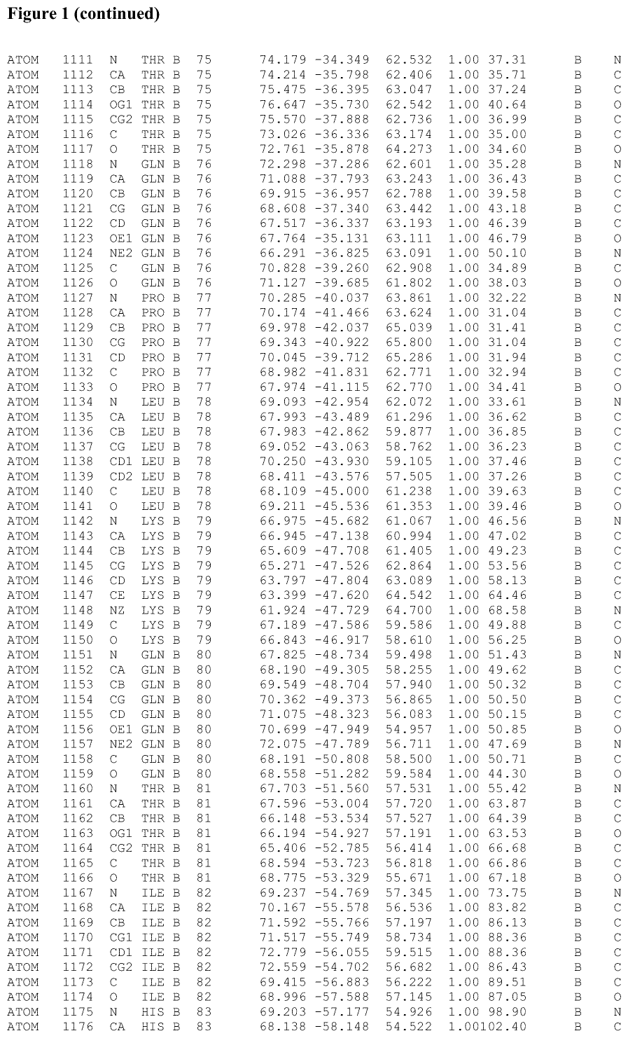

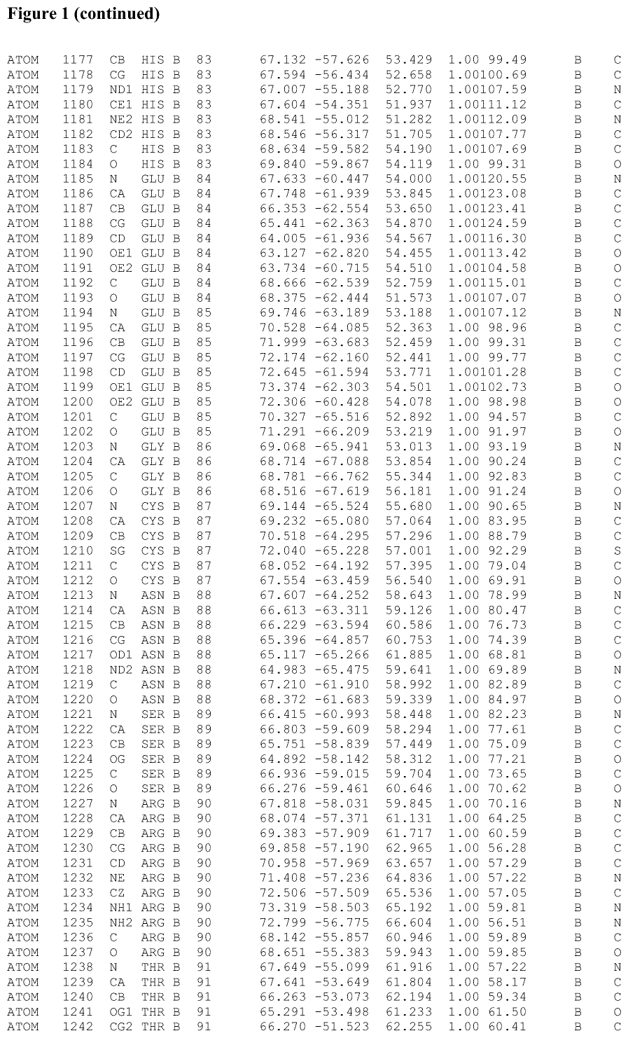

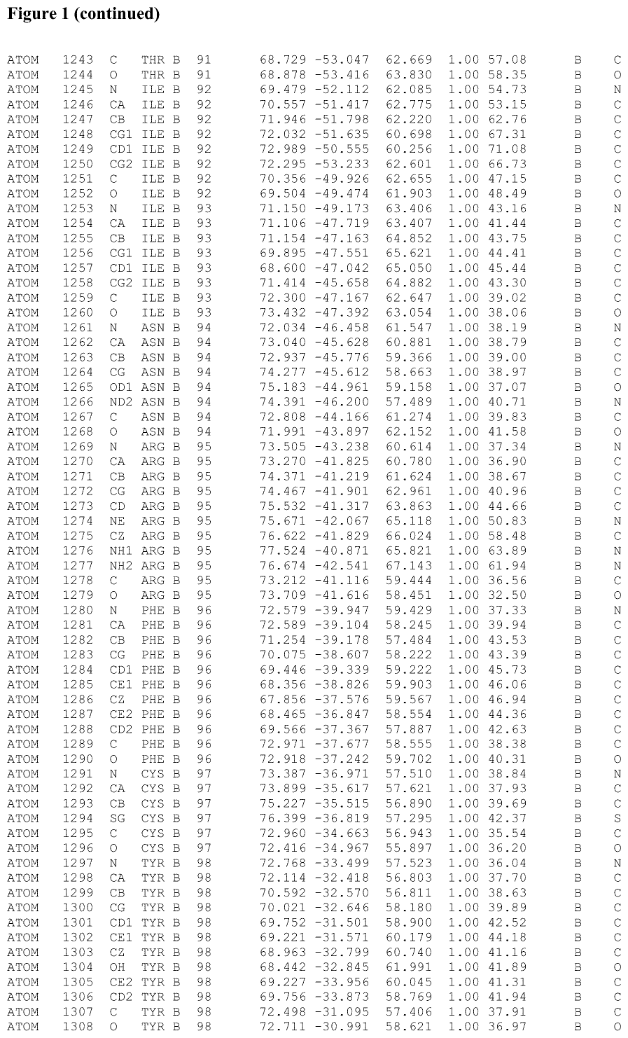

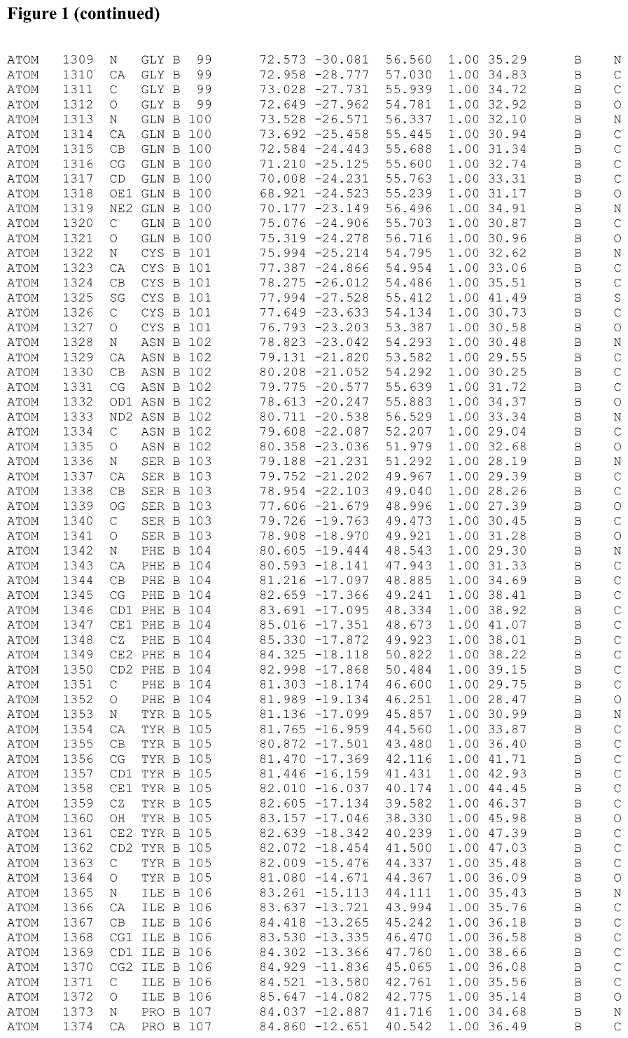

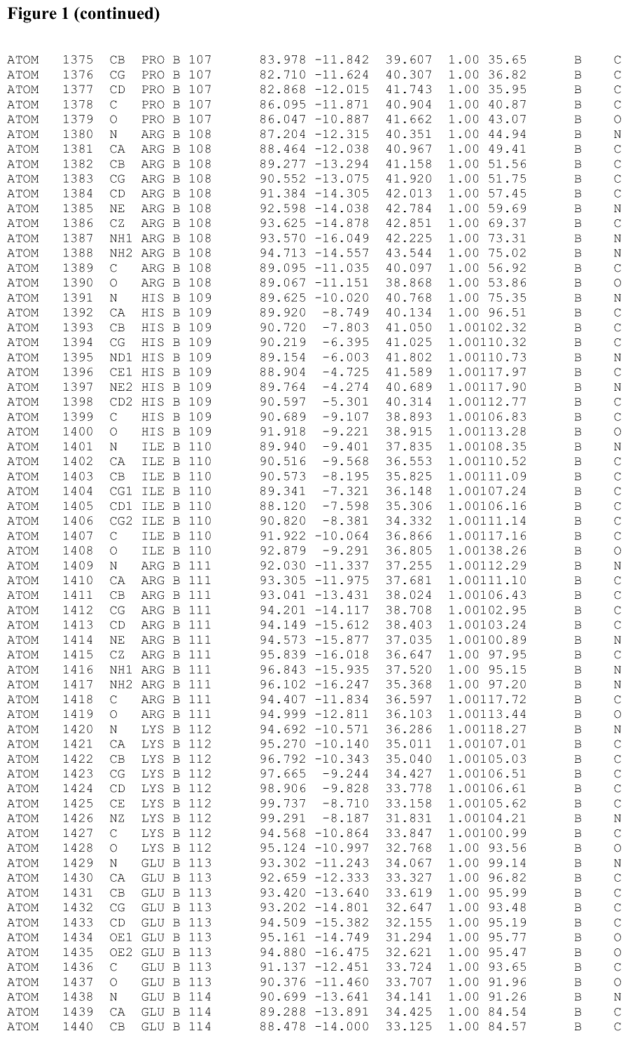

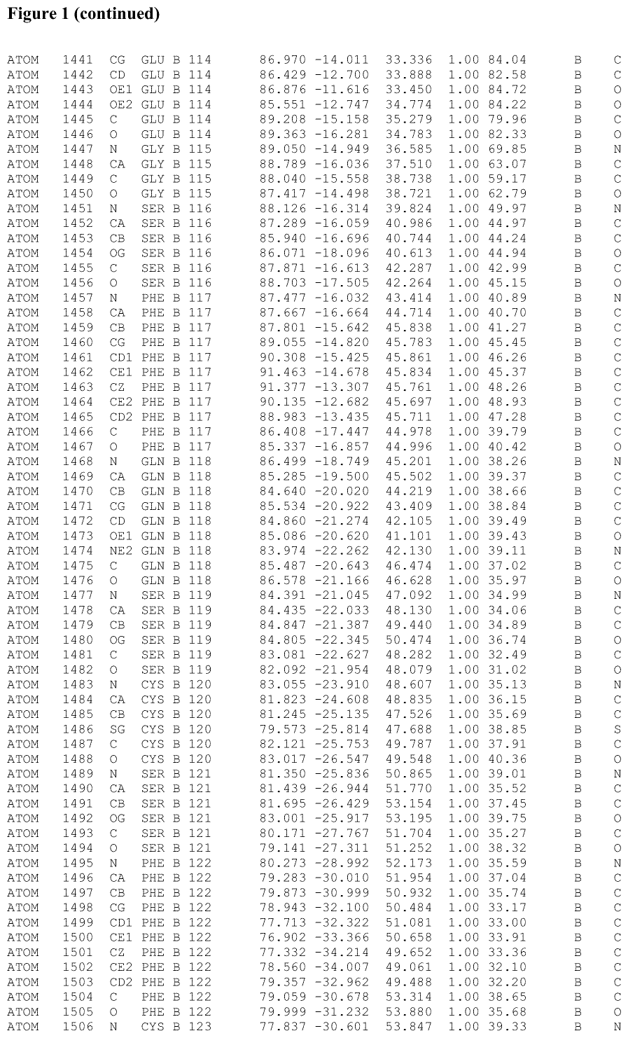

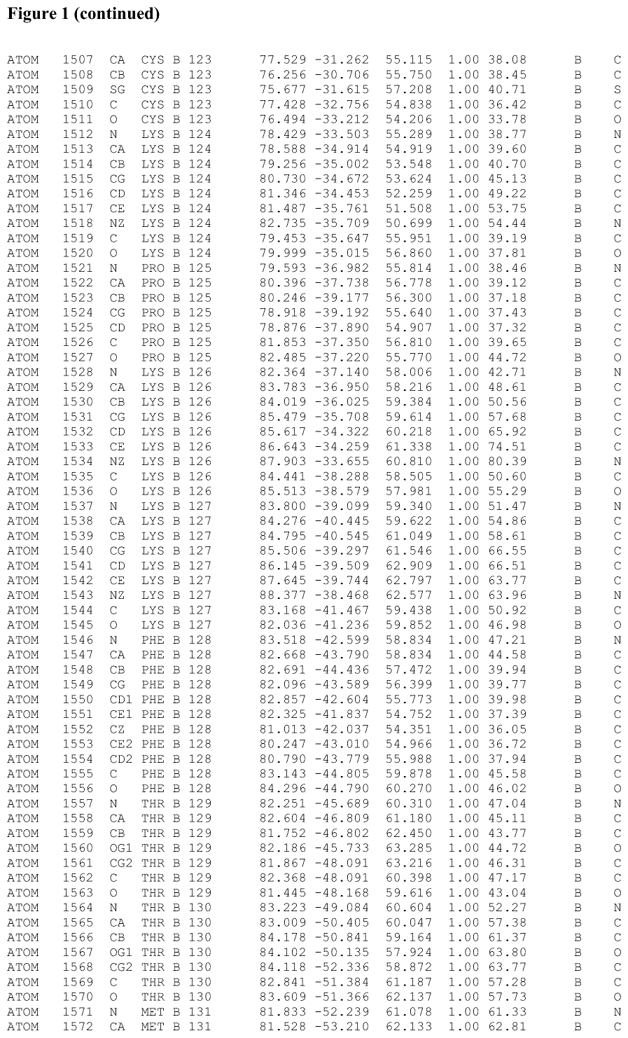

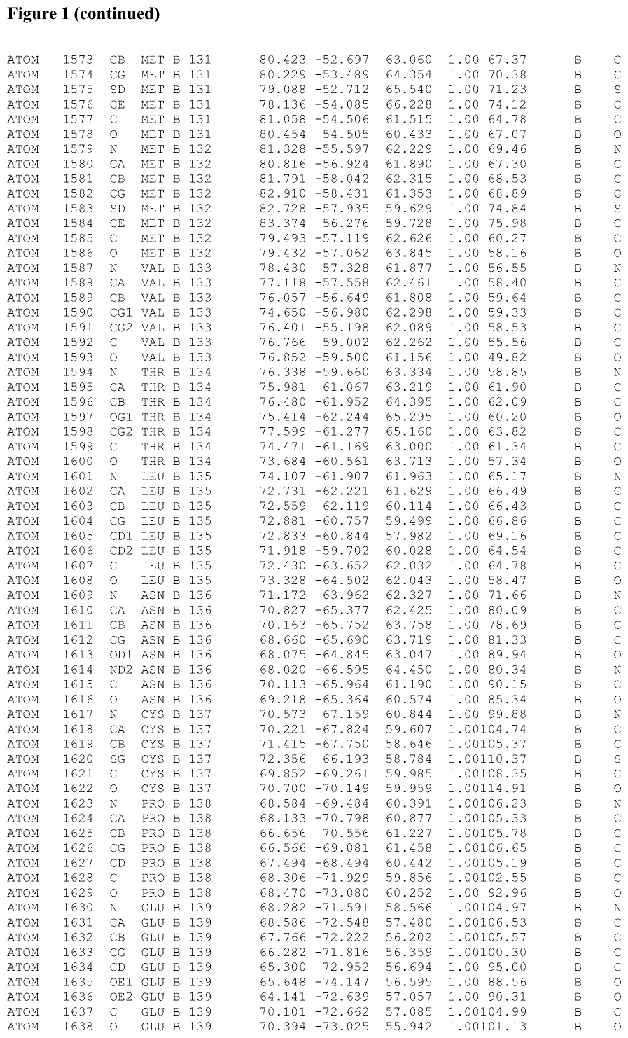

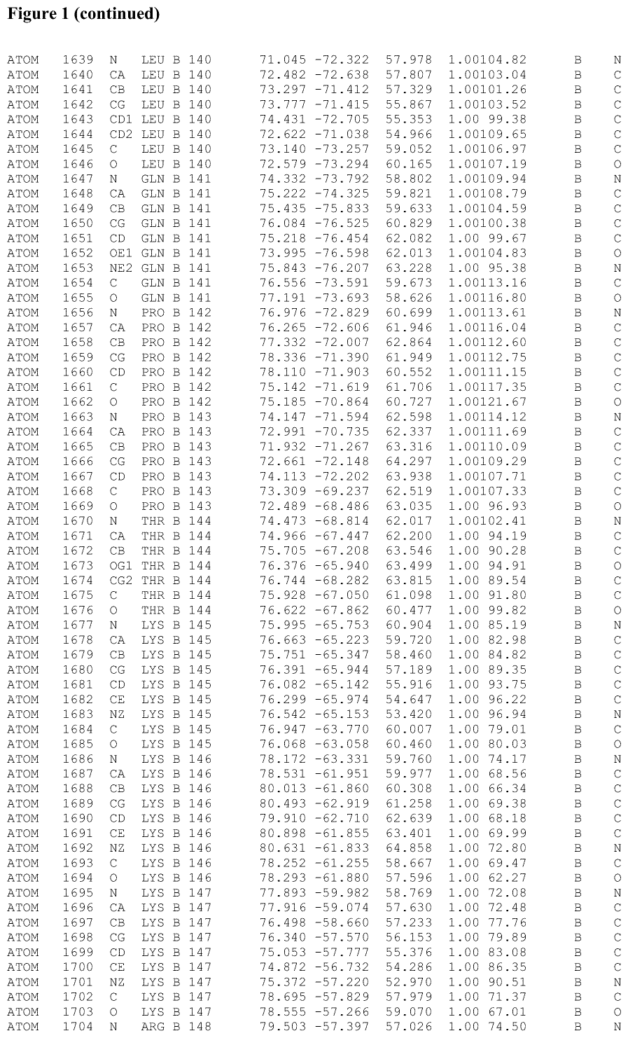

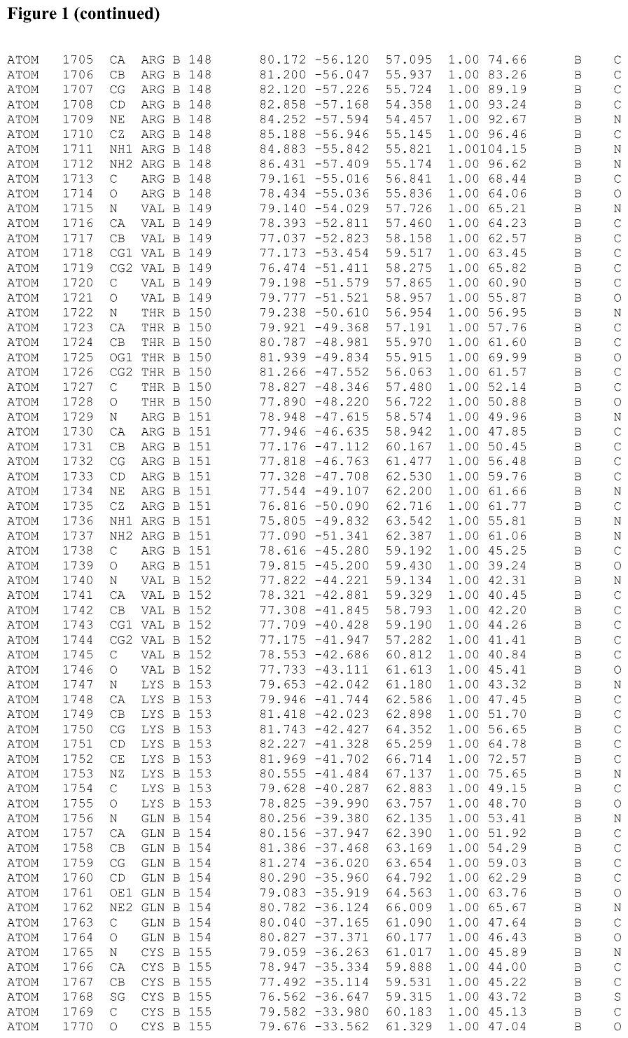

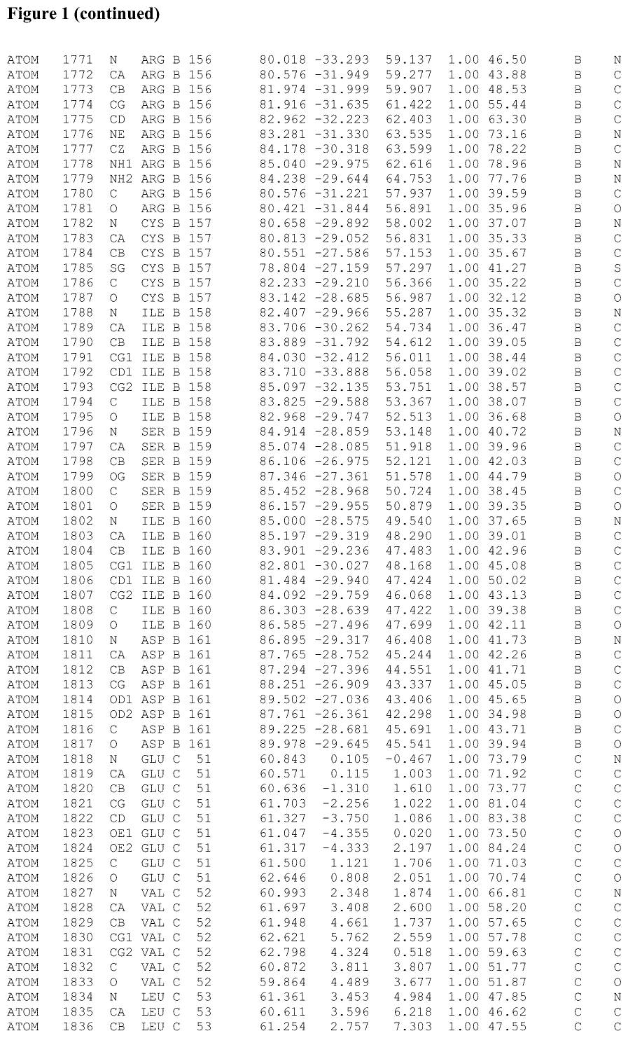

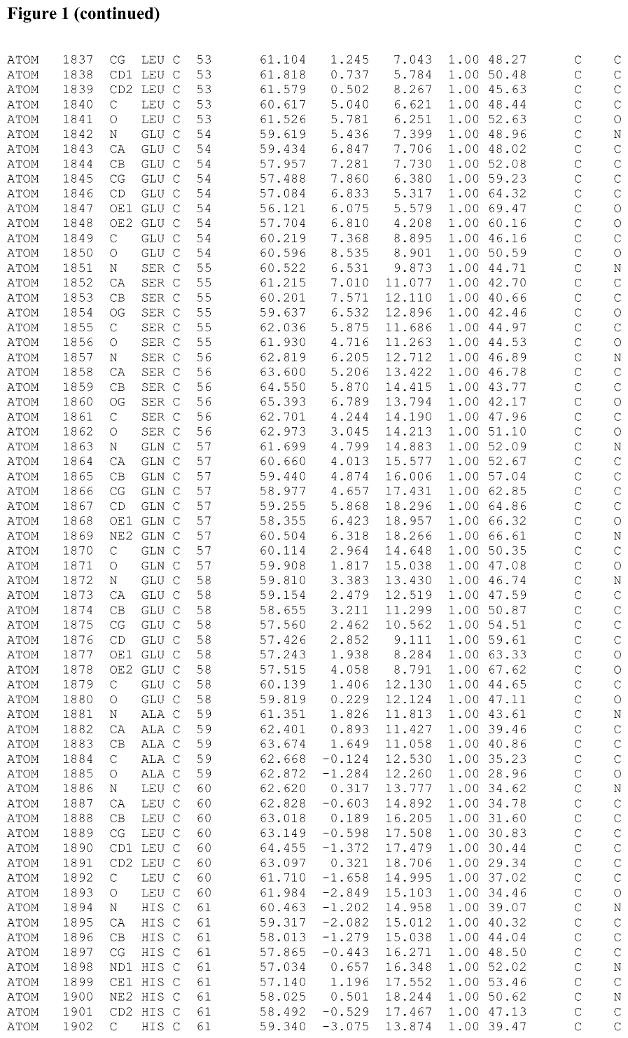

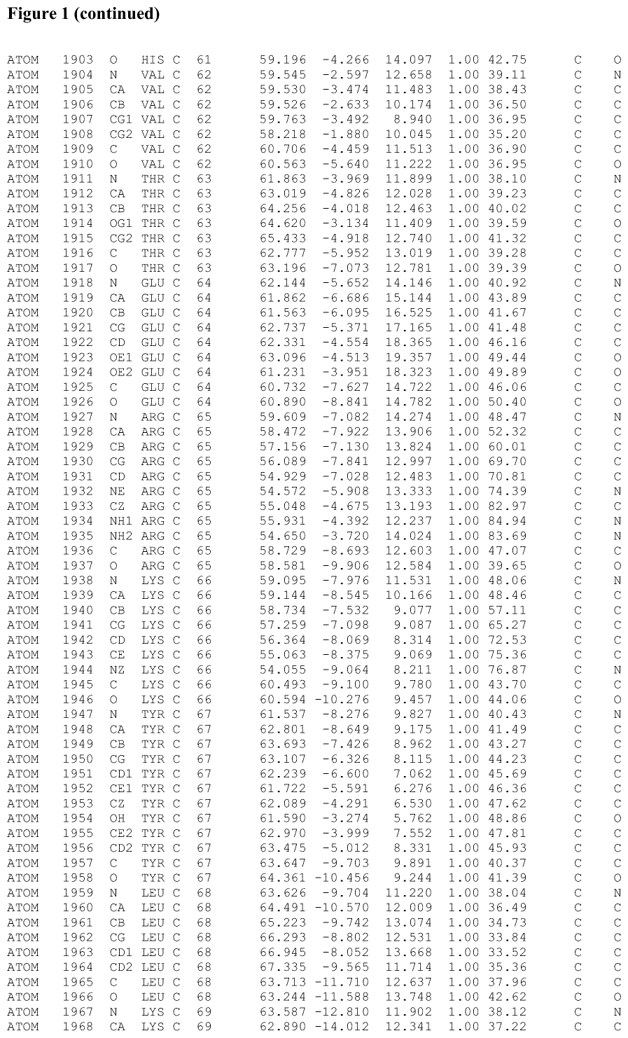

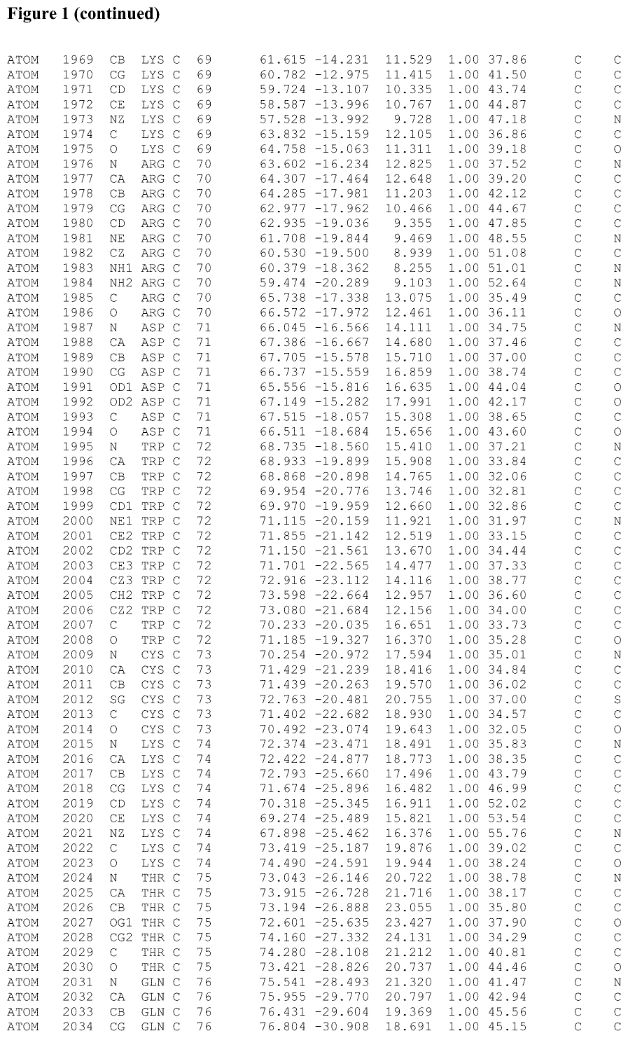

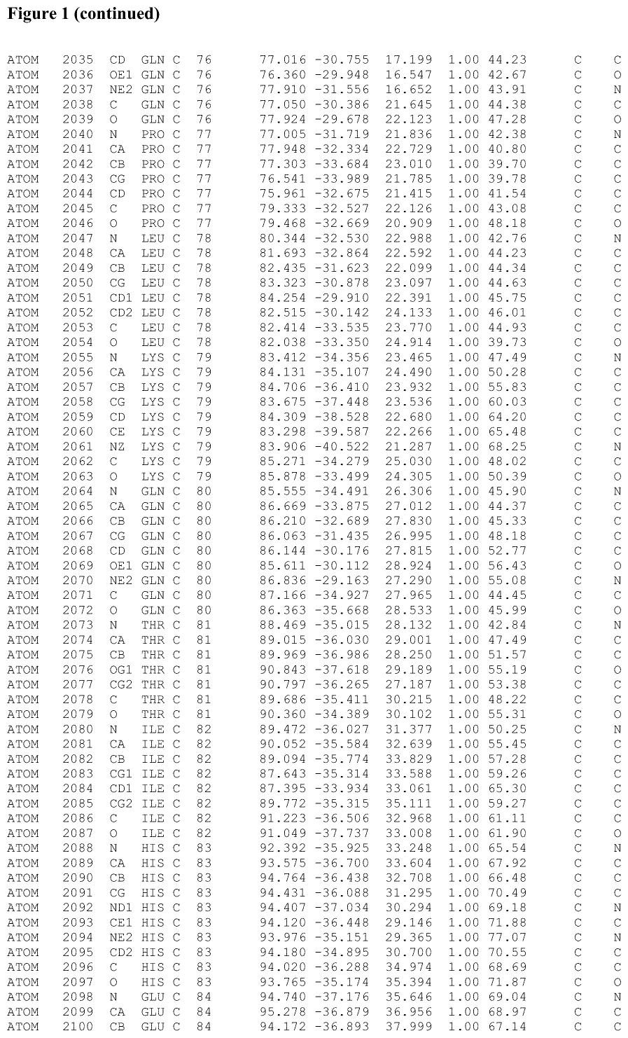

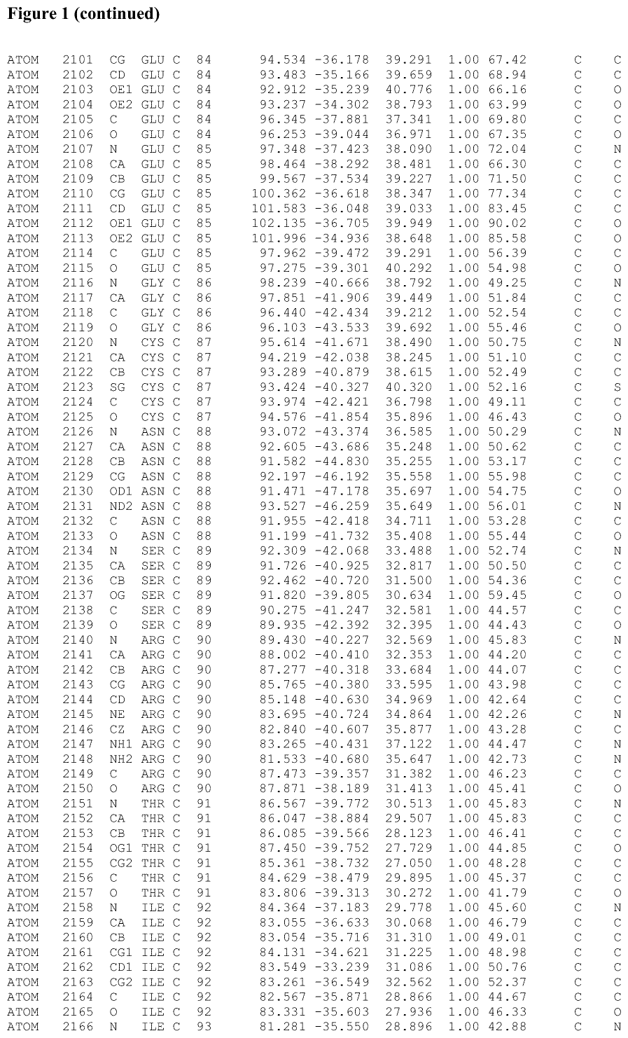

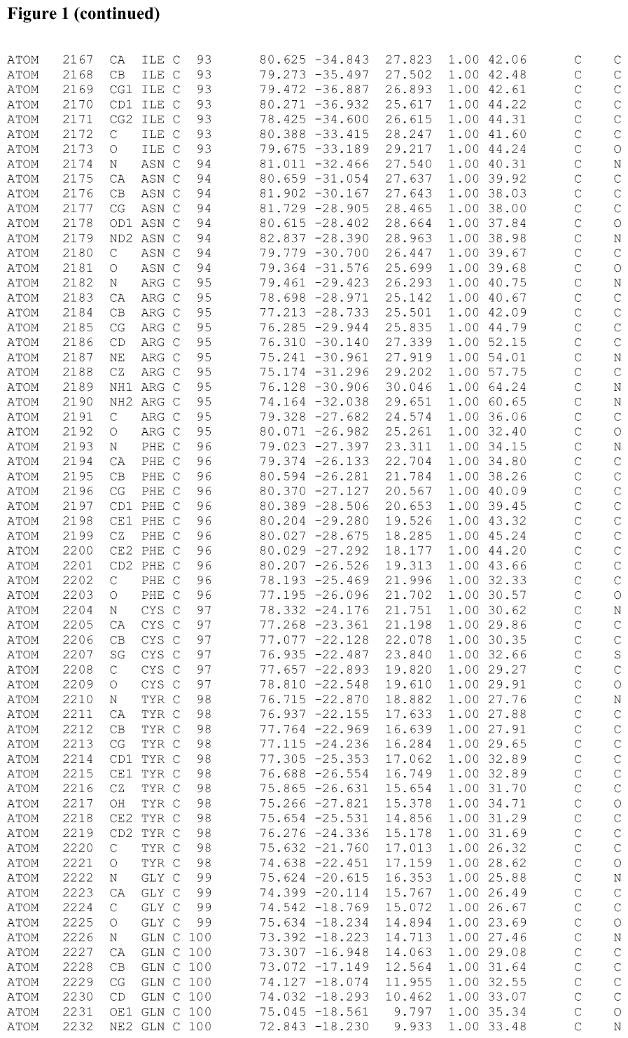

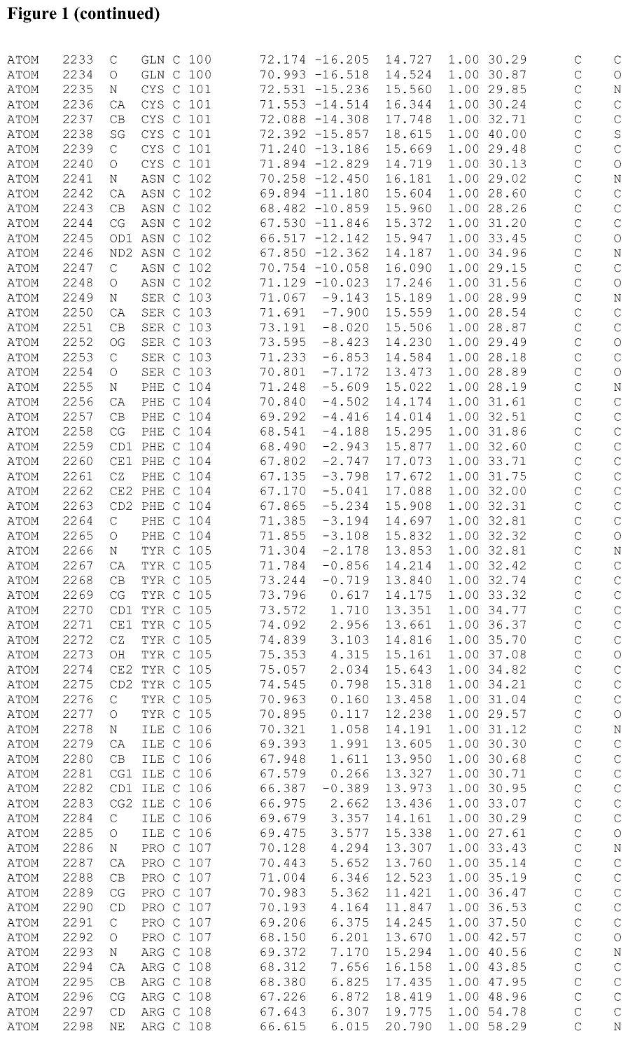

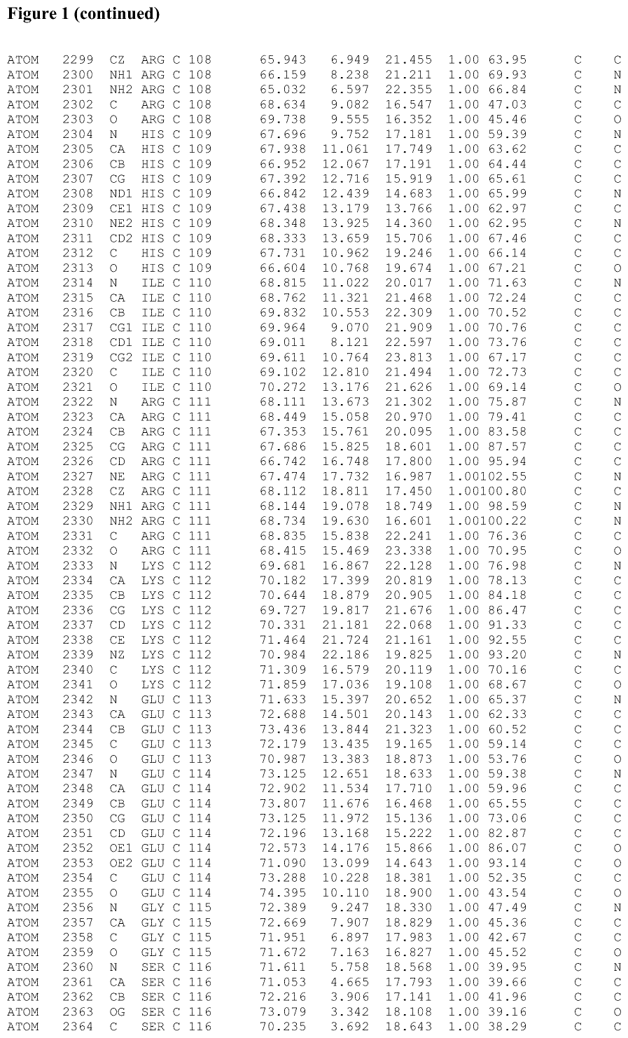

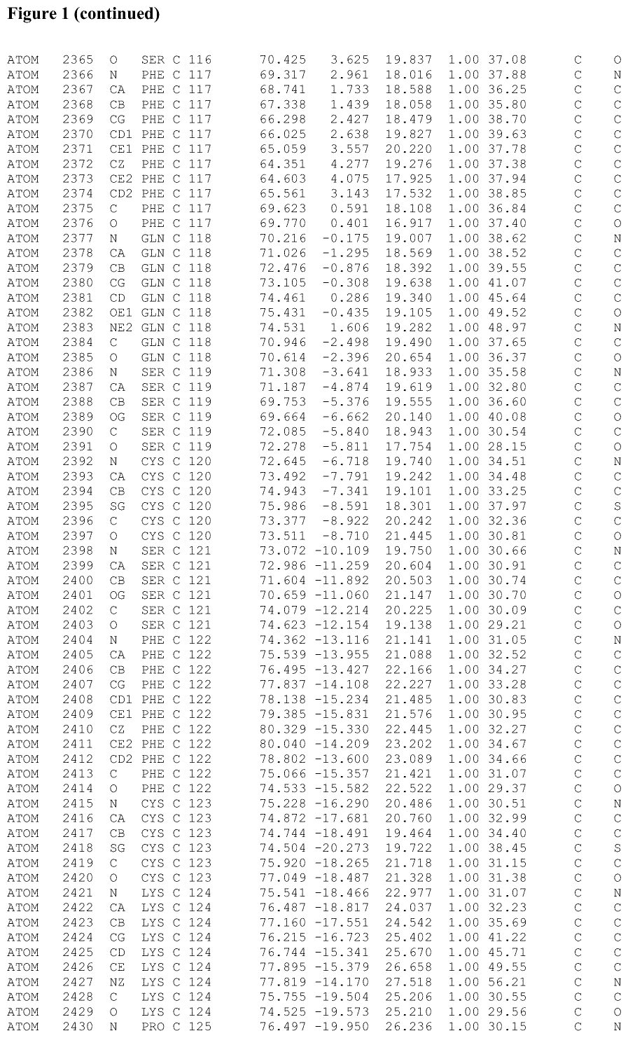

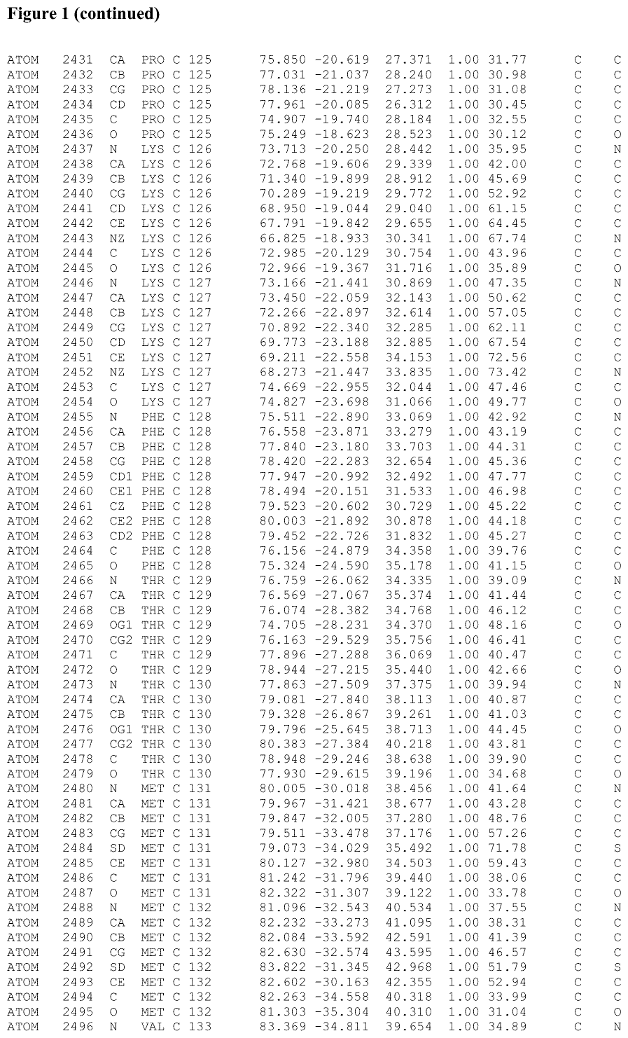

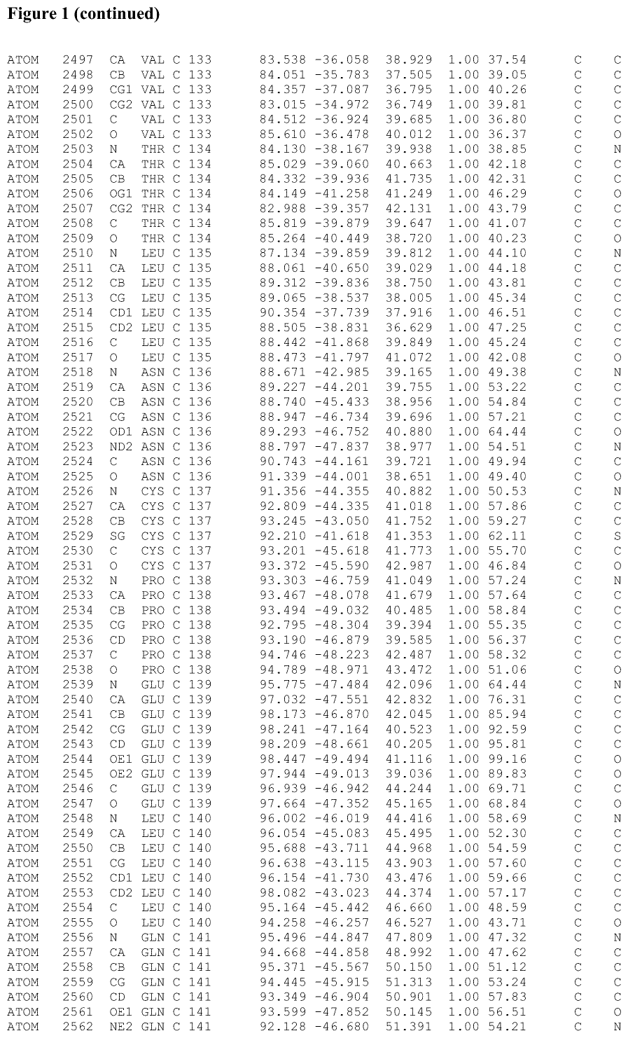

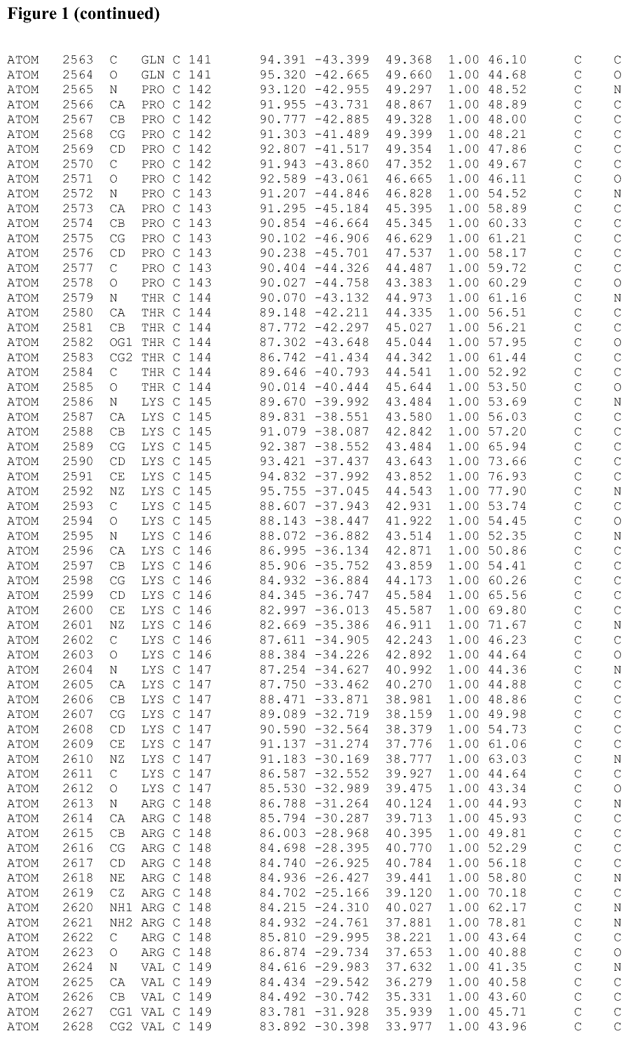

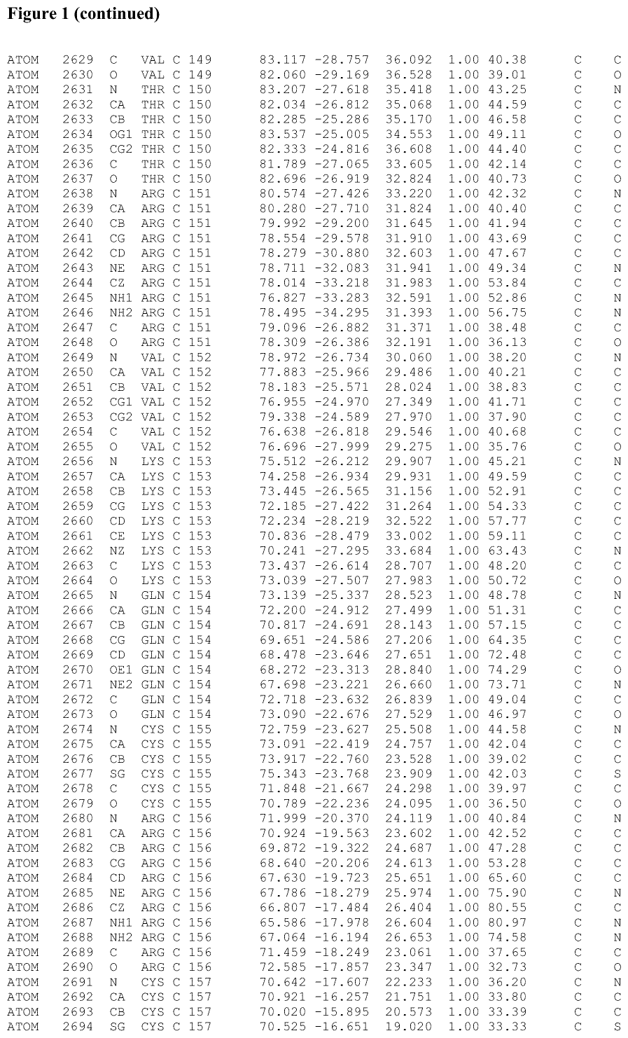

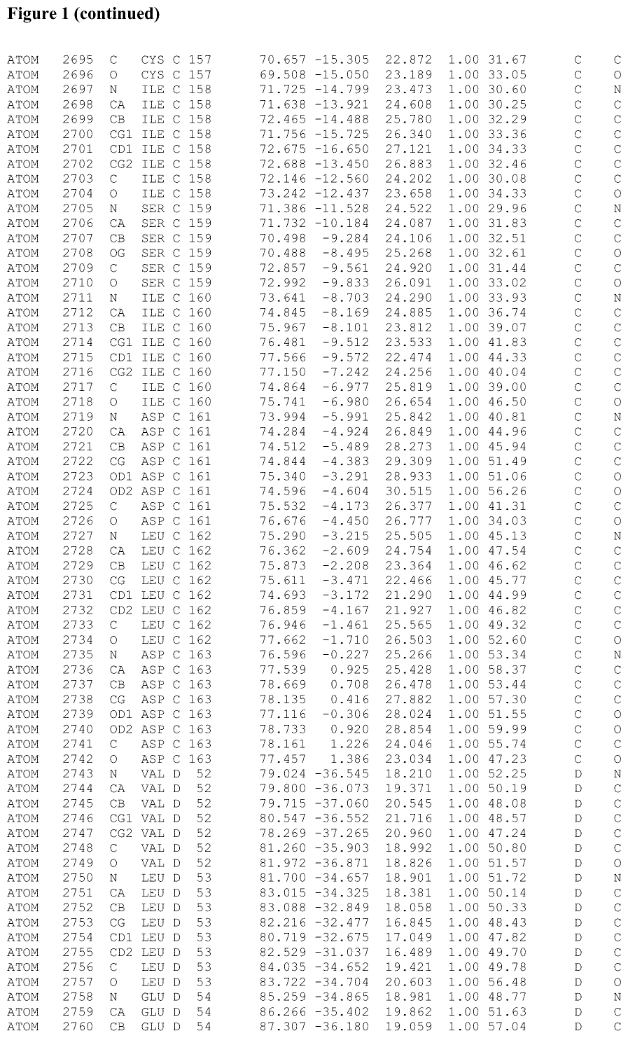

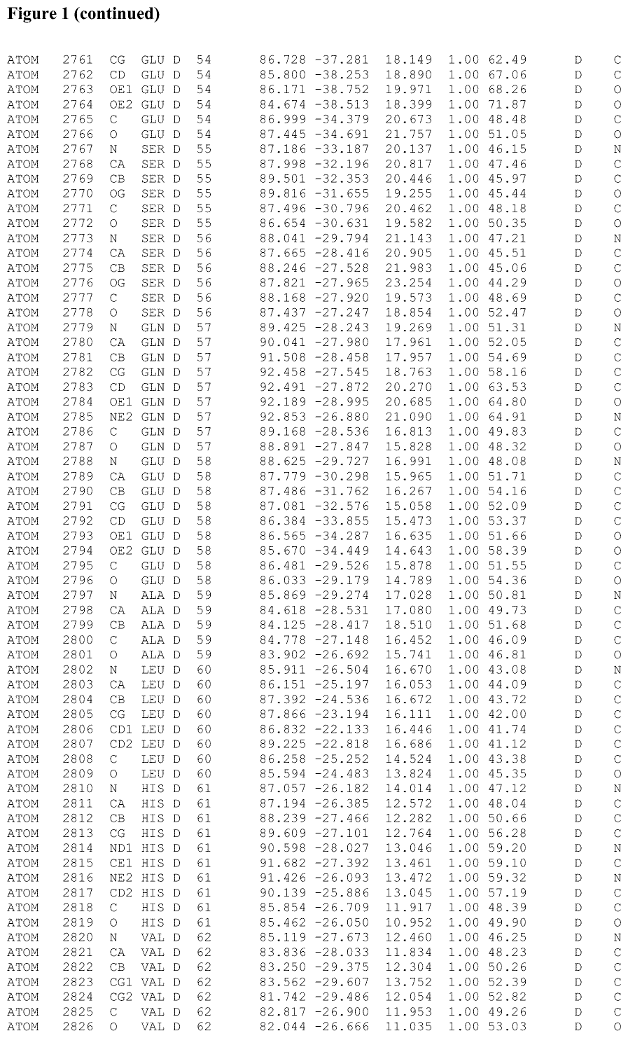

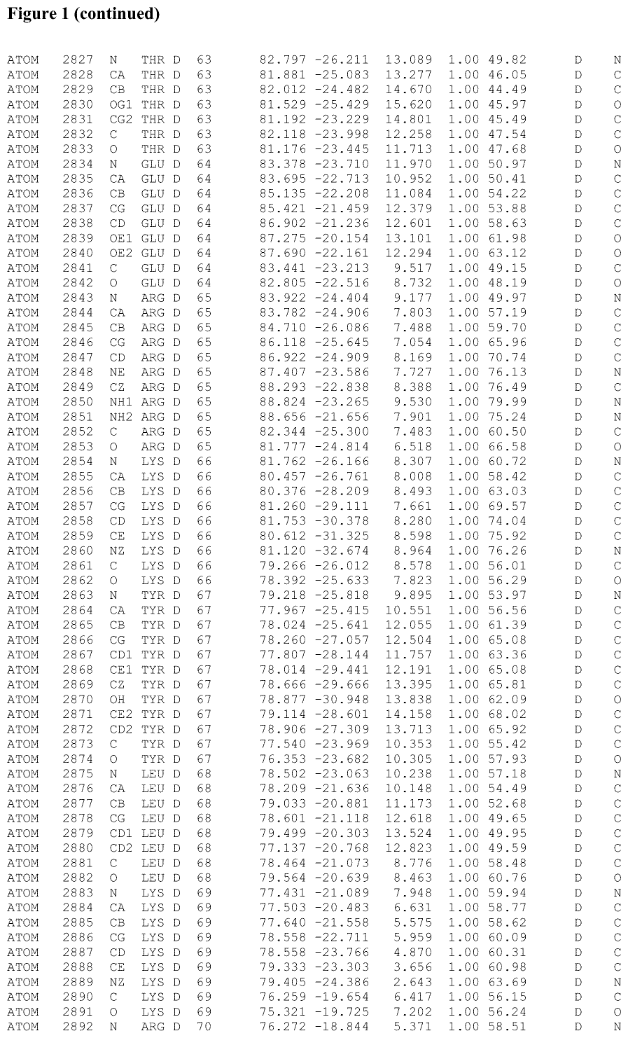

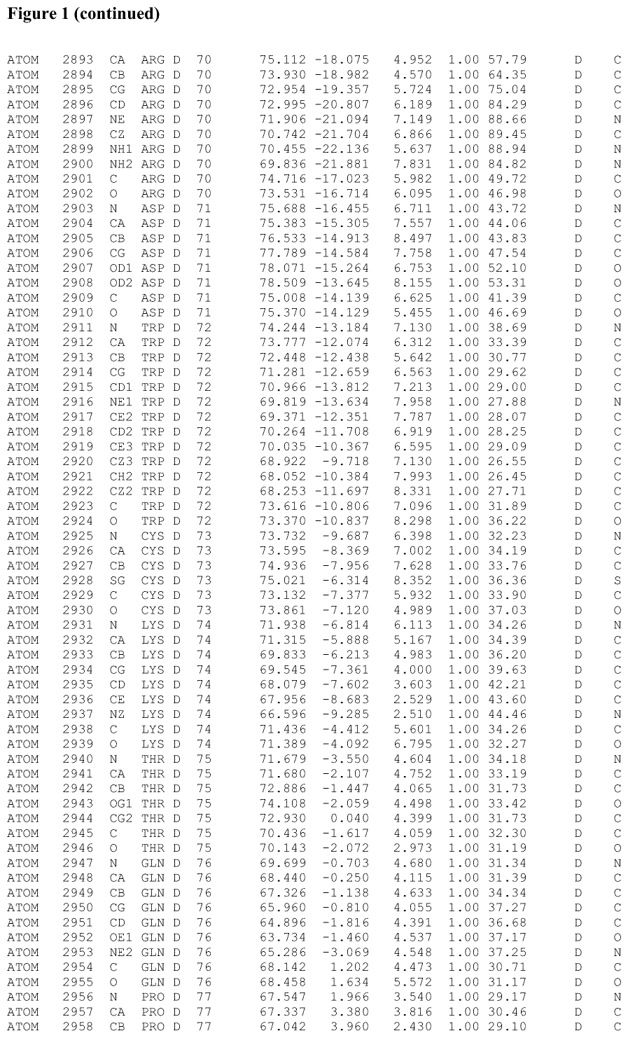

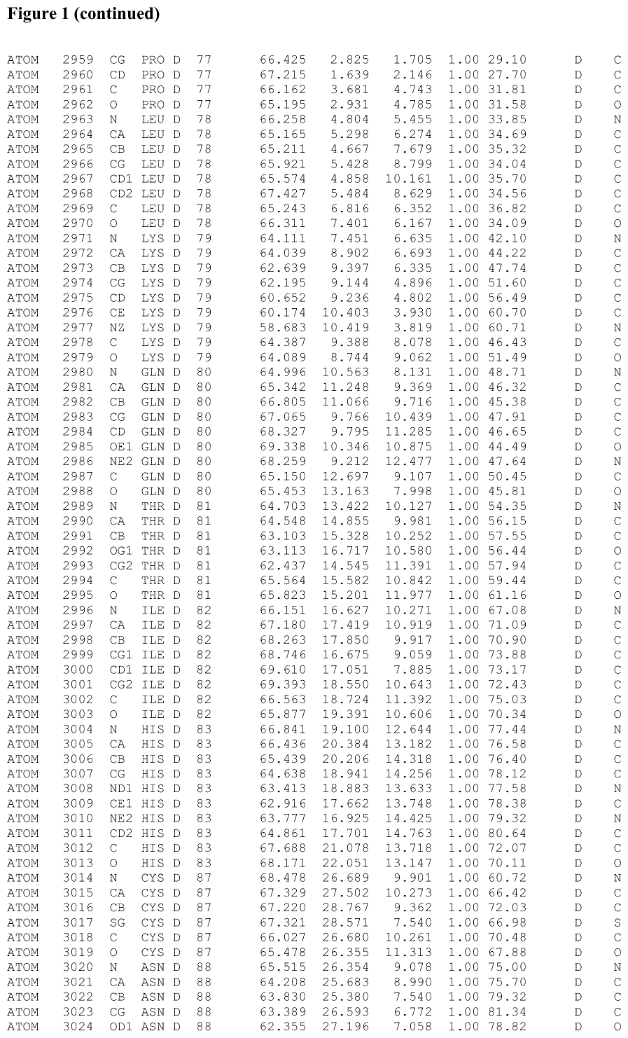

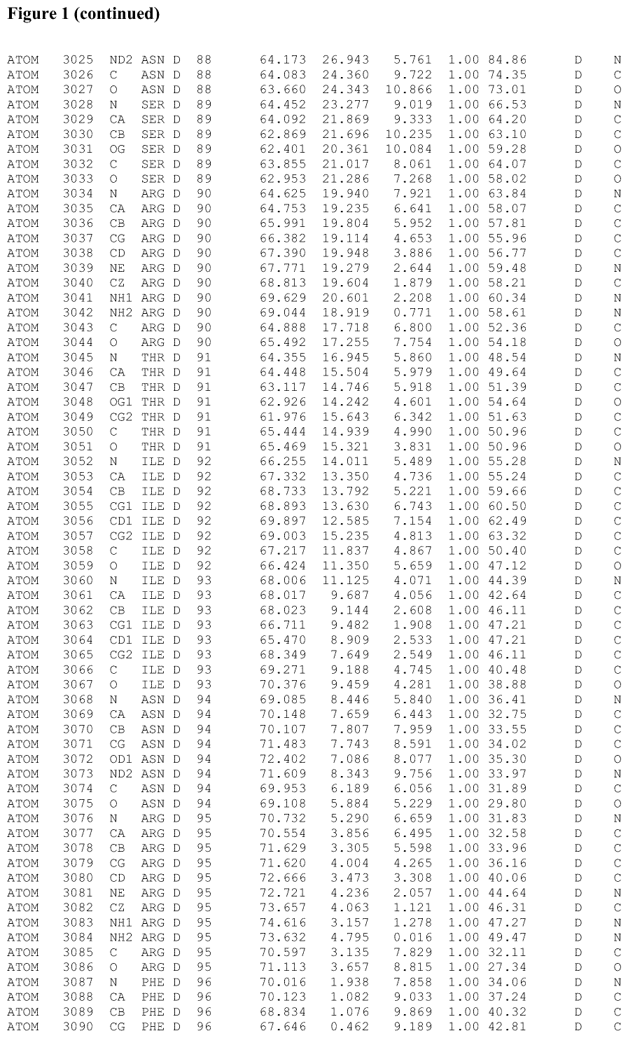

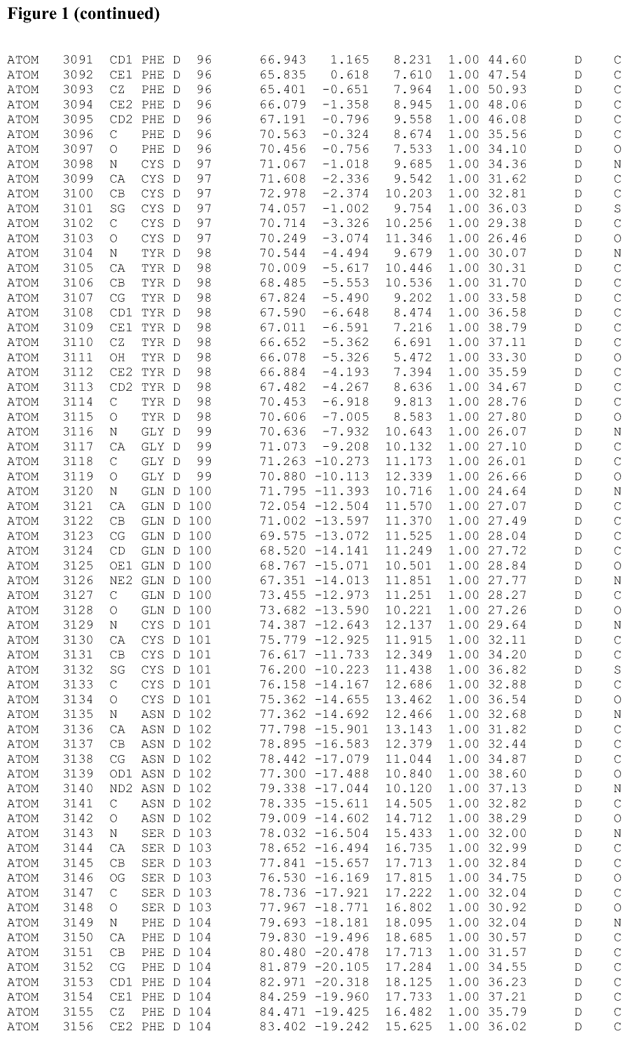

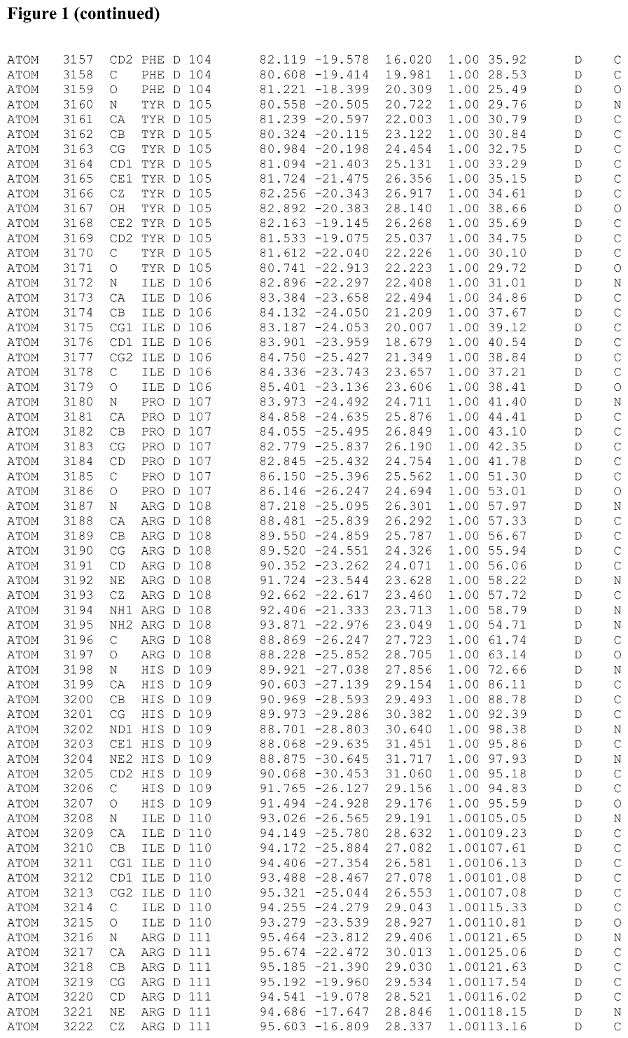

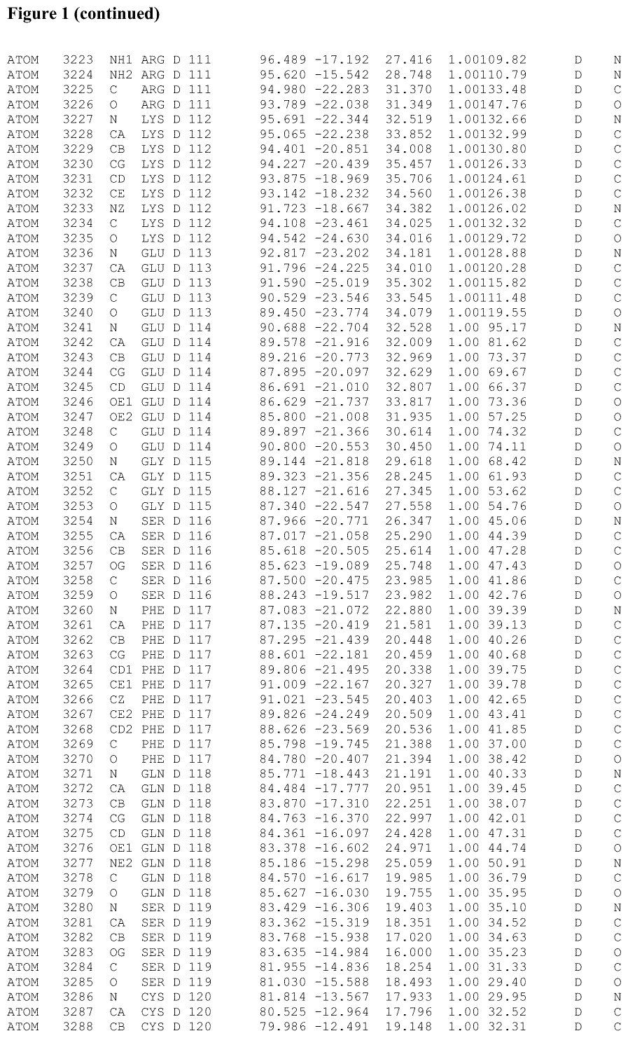

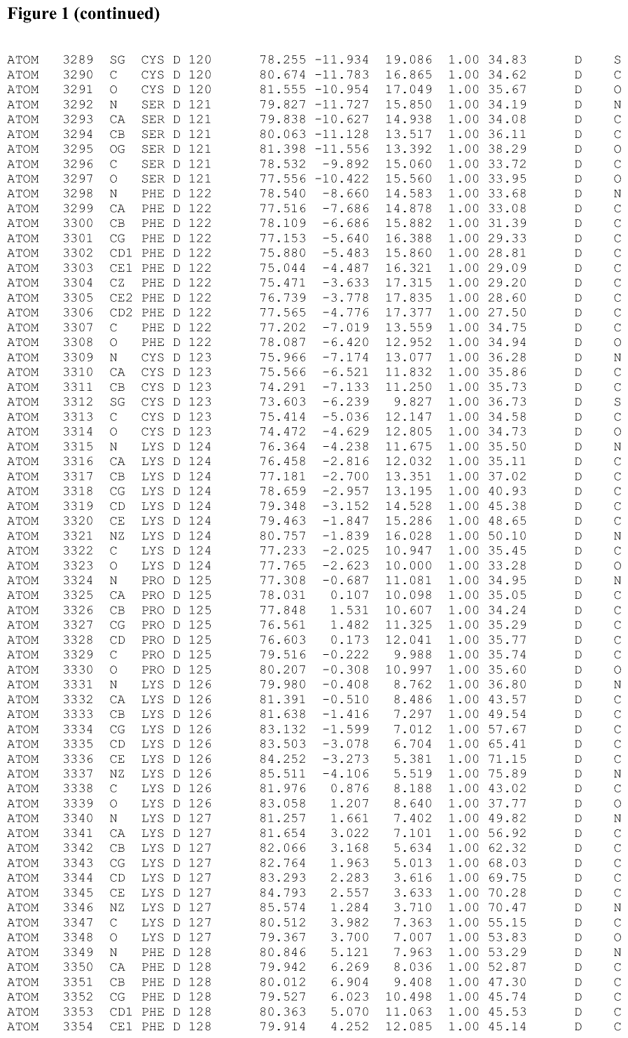

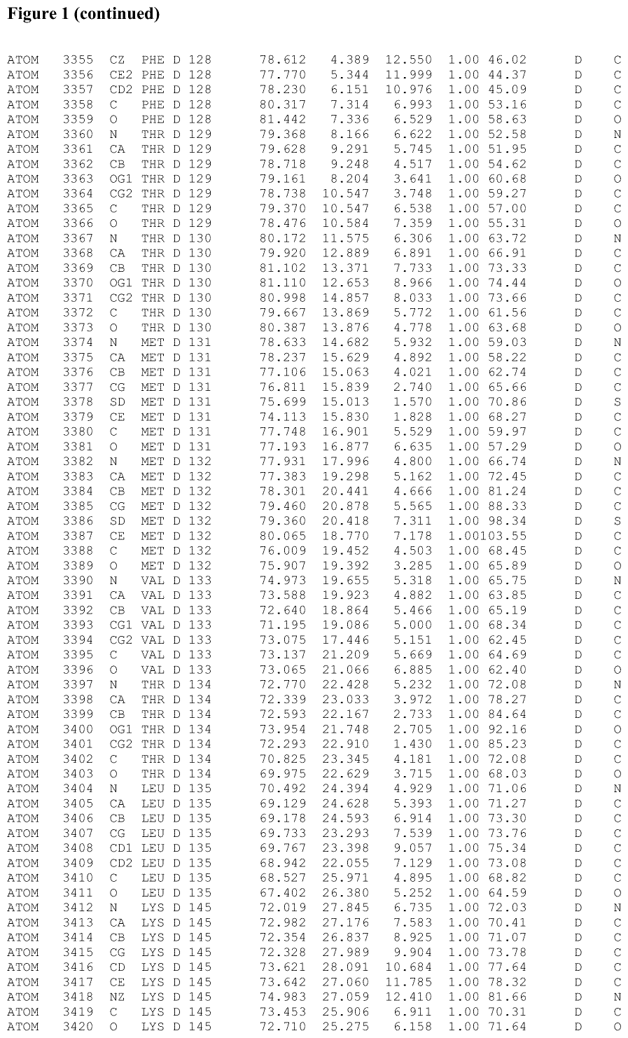

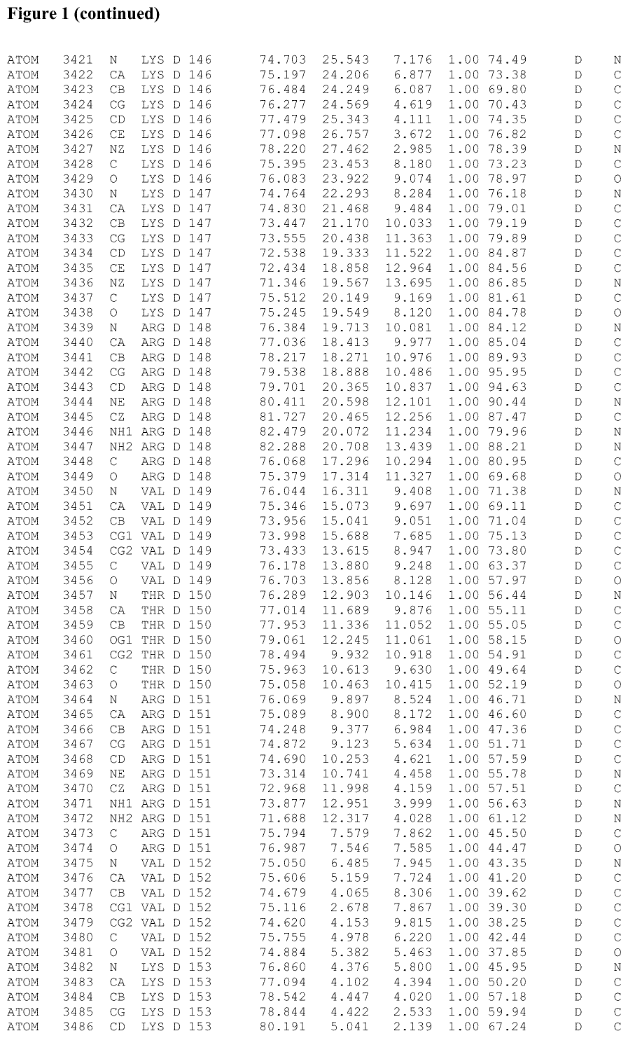

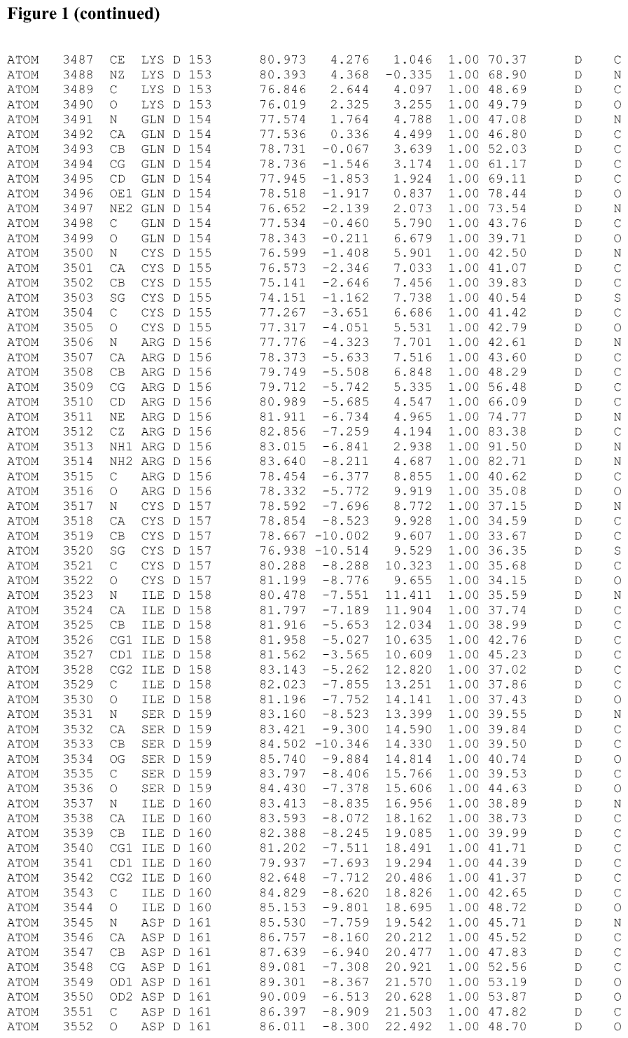

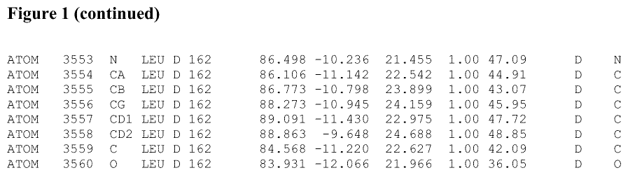

The present invention provides the structural coordinates of human Gremlin-1. The complete coordinates are listed in FIG. 1 (Table 1).

The present invention also provides a crystal of human Gremlin-1, consisting of a C2 space group with unit cell dimensions of a=84.55 Å, b=107.22 Å and c=77.09 Å.

The present invention further provides for a crystal of Gremlin-1 in complex with an antibody, more specifically a Fab with a heavy chain of SEQ ID NO: 18 and a light chain of SEQ ID NO: 19.

The invention further provides a machine readable data storage medium which comprises data storage material encoded with machine readable data defined by the structure coordinates of Gremlin-1 in Table 1 or coordinates defining homologues of the structure.

The invention provides for use of the structural data in table 1, and the machine readable data storage medium, as a structural model for Gremlin-1. Such a structural model may be used to screen for agents that interact with Gremlin-1. The screening may be high throughput screening.

An agent that interacts with Gremlin-1 is typically an agent which binds Gremlin-1. Agents that interact with Gremlin-1 may modulate Gremlin-1. An inhibitory modulating agent may have an effect on any of the functions of Gremlin-1, but typically reduces binding of Gremlin-1 to BMP (BMP 2/4/7). Gremlin-1 is a negative regulator of BMP, so reduced binding increases signalling through BMP. An activating modulating agent may increase binding of Gremlin-1 to BMP.

BMP binding and signalling may be detected by any method known in the art. For example, the Examples of the present application describe a SMAD phosphorylation assay. SMAD1, 5 and 8 are phosphorylated upon BMP signalling. An increase in SMAD phosphorylation may therefore be used to determine increased BMP signalling, which may reflect a reduction in binding to Gremlin-1.

The Examples also describe an Id1 reporter gene assay, where the Id1 gene is a target gene of BMP signalling. An increase in recovery of the signal in this assay may therefore also be used to determine if an agent inhibits Gremlin-1 binding to BMP.

An agent as referred to herein could be any molecule which could potentially interact with Gremlin-1, but is preferably a small molecule or antibody.

The invention also provides a method of screening for modulatory agents of Gremlin-1 activity, comprising the steps of:
(a) identifying a ligand binding site from the structural coordinates in Table 1;
(b) identifying candidate agents which interact with at least part of the ligand binding site; and
(c) obtaining or synthesising said agent.

The ligand binding site could be any putative site on Gremlin-1 which interacts with a protein (ligand). The ligand binding site is typically the BMP binding site. As shown in the Examples, the present inventors have identified a putative BMP binding site based on the Gremlin-1 crystal structure. This binding site comprises the following amino acids: Trp93, Phe117, Tyr119, Phe125, Tyr126 and Phe138, wherein the residue numbering is based on SEQ ID NO: 1.

The screening method of the invention may therefore comprise identifying agents which interact with one or more of these residues, preferably at least 2, 3, 4 or all 6 of these residues.

Interaction of an agent with protein residues may be determined by any appropriate method known in the art, such as distances between the residue and agent as determined by x-ray crystallography (typically less than 6 Å, or less than 4 Å). As discussed in the Examples below, the region of Gremlin-1 which may be targeted by a therapeutic may include amino acids Asp92-Leu99, Arg116-His130, Ser137-Ser142, Cys176-Cys178. These are within 6 Å of those mutated on the surface of Gremlin-1.

Steps (a) and (b) of the screening method are typically performed in silico, and the agent may be obtained and synthesised by any method known in the art.

In one embodiment, the present invention provides an antibody which binds to an epitope on Gremlin-1 comprising at least one residue selected from Trp93, Phe117, Tyr119, Phe125, Tyr126 and Phe138, wherein the residue numbering is according to SEQ ID NO: 1. The present invention also provides an antibody, which binds an epitope comprising all of Trp93, Phe117, Tyr119, Phe125, Tyr126 and Phe138.

The invention also provides for use of this BMP binding region of Gremlin-1 for generating (potentially inhibitory) antibodies. For example, the invention provides an antigen comprising at least one (preferably all) of the residues listed above, which can be used for antibody generation.

Instead of interacting with the BMP binding site of Gremlin-1, an agent may act allosterically. Here, an agent binds away from the normal binding site but is still capable of modulating the activity of Gremlin-1 e.g. through induced conformational changes in the protein. The structural model and screening method of the invention may therefore also be used to identify allosteric modulators of Gremlin-1.

The Ab 7326 antibody of the invention has been found to act allosterically. The epitope of this antibody comprises the following residues: Ile131, Lys147, Lys148, Phe149, Thr150, Thr151, Arg169, Lys174 and Gln175, wherein the residue numbering is based on SEQ ID NO: 1. Accordingly, the screening method of the invention may involve identifying agents which interact with at least 1, 2, 3, 4, 5 or all 9 of these residues. Such agents can then be tested e.g. using the assays described in the Examples for inhibition of BMP binding. Preferably, Lys147, Lys148, Phe149, Thr150, Thr151, Arg169, Lys174 and Gln175 are located on one monomer of Gremlin-1 and Ile131 is located on the other monomer of Gremlin-1 (Gremlin-1 dimers bind to BMP dimers).

Once again, the invention also encompasses an antigen comprising at least one (preferably all) of these residues for producing anti-Gremlin-1 antibodies.

Again, agents may be identified as interacting with these residues by any appropriate method known in the art. The agent is preferably a small molecule or antibody.

### Antibodies

The present invention provides antibodies that bind Gremlin-1.

The term “antibody” as referred to herein includes whole antibodies and any antigen binding fragment (i.e., “antigen-binding portion”) or single chains thereof. An antibody refers to a glycoprotein comprising at least two heavy (H) chains and two light (L) chains inter-connected by disulfide bonds, or an antigen-binding portion thereof. Each heavy chain is comprised of a heavy chain variable region (abbreviated herein as HCVR or V_{H}) and a heavy chain constant region. Each light chain is comprised of a light chain variable region (abbreviated herein as LCVR or V_{L}) and a light chain constant region. The variable regions of the heavy and light chains contain a binding domain that interacts with an antigen. The V_{H} and V_{L} regions can be further subdivided into regions of hypervariability, termed complementarity determining regions (CDR), interspersed with regions that are more conserved, termed framework regions (FR).

The constant regions of the antibodies may mediate the binding of the immunoglobulin to host tissues or factors, including various cells of the immune system (e.g., effector cells) and the first component (C1q) of the classical complement system.

An antibody of the invention may be a monoclonal antibody or a polyclonal antibody, and will typically be a monoclonal antibody. An antibody of the invention may be a chimeric antibody, a CDR-grafted antibody, a nanobody, a human or humanised antibody or an antigen-binding portion of any thereof. For the production of both monoclonal and polyclonal antibodies, the experimental animal is typically a non-human mammal such as a goat, rabbit, rat or mouse but the antibody may also be raised in other species.

Polyclonal antibodies may be produced by routine methods such as immunisation of a suitable animal, with the antigen of interest. Blood may be subsequently removed from the animal and the IgG fraction purified.

Antibodies against Gremlin-1 may be obtained, where immunisation of an animal is necessary, by administering the polypeptides to an animal, e.g. a non-human animal, using well-known and routine protocols, see for example Handbook of Experimental Immunology, D. M. Weir (ed.), Vol 4, Blackwell Scientific Publishers, Oxford, England, 1986). Many warm-blooded animals, such as rabbits, mice, rats, sheep, cows, camels or pigs may be immunized. However, mice, rabbits, pigs and rats are generally most suitable.

Monoclonal antibodies may be prepared by any method known in the art such as the hybridoma technique (Kohler & Milstein, 1975, Nature, 256:495-497), the trioma technique, the human B-cell hybridoma technique (Kozbor et al., 1983, Immunology Today, 4:72) and the EBV-hybridoma technique (Cole et al., Monoclonal Antibodies and Cancer Therapy, pp 77-96, Alan R Liss, Inc., 1985).

Antibodies of the invention may also be generated using single lymphocyte antibody methods by cloning and expressing immunoglobulin variable region cDNAs generated from single lymphocytes selected for the production of specific antibodies by for example the methods described by Babcook, J. et al., 1996, Proc. Natl. Acad. Sci. USA 93(15): 7843-78481; WO92/02551; WO2004/051268 and WO2004/106377.

The antibodies of the present invention can also be generated using various phage display methods known in the art and include those disclosed by Brinkman et al. (in J. Immunol. Methods, 1995, 182: 41-50), Ames et al. (J. Immunol. Methods, 1995, 184:177-186), Kettleborough et al. (Eur. J. Immunol. 1994, 24:952-958), Persic et al. (Gene, 1997 187 9-18), Burton et al. (Advances in Immunology, 1994, 57:191-280) and WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

Fully human antibodies are those antibodies in which the variable regions and the constant regions (where present) of both the heavy and the light chains are all of human origin, or substantially identical to sequences of human origin, but not necessarily from the same antibody. Examples of fully human antibodies may include antibodies produced, for example by the phage display methods described above and antibodies produced by mice in which the murine immunoglobulin variable and optionally the constant region genes have been replaced by their human counterparts e.g. as described in general terms in EP 0546073, U.S. Pat. Nos. 5,545,806, 5,569,825, 5,625,126, 5,633,425, 5,661,016, 5,770,429, EP 0438474 and EP 0463151.

Alternatively, an antibody according to the invention may be produced by a method comprising immunising a non-human mammal with a Gremlin-1 immunogen; obtaining an antibody preparation from said mammal; deriving therefrom monoclonal antibodies that recognise Gremlin-1.

The antibody molecules of the present invention may comprise a complete antibody molecule having full length heavy and light chains or a fragment or antigen-binding portion thereof. The term “antigen-binding portion” of an antibody refers to one or more fragments of an antibody that retain the ability to selectively bind to an antigen. It has been shown that the antigen-binding function of an antibody can be performed by fragments of a full-length antibody. The antibodies and fragments and antigen binding portions thereof may be, but are not limited to Fab, modified Fab, Fab′, modified Fab′, F(ab′)2, Fv, single domain antibodies (e.g. VH or VL or VHH), scFv, bi, tri or tetra-valent antibodies, Bis-scFv, diabodies, triabodies, tetrabodies and epitope-binding fragments of any of the above (see for example Holliger and Hudson, 2005, Nature Biotech. 23(9):1126-1136; Adair and Lawson, 2005, Drug Design Reviews—Online 2(3), 209-217). The methods for creating and manufacturing these antibody fragments are well known in the art (see for example Verma et al., 1998, Journal of Immunological Methods, 216, 165-181). Other antibody fragments for use in the present invention include the Fab and Fab′ fragments described in International patent applications WO 2005/003169, WO 2005/003170 and WO 2005/003171 and Fab-dAb fragments described in International patent application WO2009/040562. Multi-valent antibodies may comprise multiple specificities or may be monospecific (see for example WO 92/22853 and WO 05/113605). These antibody fragments may be obtained using conventional techniques known to those of skill in the art, and the fragments may be screened for utility in the same manner as intact antibodies.

The constant region domains of the antibody molecule of the present invention, if present, may be selected having regard to the proposed function of the antibody molecule, and in particular the effector functions which may be required. For example, the constant region domains may be human IgA, IgD, IgE, IgG or IgM domains. In particular, human IgG constant region domains may be used, especially of the IgG1 and IgG3 isotypes when the antibody molecule is intended for therapeutic uses and antibody effector functions are required. Alternatively, IgG2 and IgG4 isotypes may be used when the antibody molecule is intended for therapeutic purposes and antibody effector functions are not required.

An antibody of the invention may be prepared, expressed, created or isolated by recombinant means, such as (a) antibodies isolated from an animal (e.g., a mouse) that is transgenic or transchromosomal for the immunoglobulin genes of interest or a hybridoma prepared therefrom, (b) antibodies isolated from a host cell transformed to express the antibody of interest, e.g., from a transfectoma, (c) antibodies isolated from a recombinant, combinatorial antibody library, and (d) antibodies prepared, expressed, created or isolated by any other means that involve splicing of immunoglobulin gene sequences to other DNA sequences.

An antibody of the invention may be a human antibody or a humanised antibody. The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

Such a human antibody may be a human monoclonal antibody. Such a human monoclonal antibody may be produced by a hybridoma that includes a B cell obtained from a transgenic nonhuman animal, e.g., a transgenic mouse, having a genome comprising a human heavy chain transgene and a light chain transgene fused to an immortalized cell.

Human antibodies may be prepared by in vitro immunisation of human lymphocytes followed by transformation of the lymphocytes with Epstein-Barr virus.

The term “human antibody derivatives” refers to any modified form of the human antibody, e.g., a conjugate of the antibody and another agent or antibody.

The term “humanized antibody” is intended to refer to CDR-grafted antibody molecules in which CDR sequences derived from the germline of another mammalian species, such as a mouse, have been grafted onto human framework sequences. Additional framework region modifications may be made within the human framework sequences.

As used herein, the term ‘CDR-grafted antibody molecule’ refers to an antibody molecule wherein the heavy and/or light chain contains one or more CDRs (including, if desired, one or more modified CDRs) from a donor antibody (e.g. a murine or rat monoclonal antibody) grafted into a heavy and/or light chain variable region framework of an acceptor antibody (e.g. a human antibody). For a review, see Vaughan et al, Nature Biotechnology, 16, 535-539, 1998. In one embodiment rather than the entire CDR being transferred, only one or more of the specificity determining residues from any one of the CDRs described herein above are transferred to the human antibody framework (see for example, Kashmiri et al., 2005, Methods, 36, 25-34). In one embodiment only the specificity determining residues from one or more of the CDRs described herein above are transferred to the human antibody framework. In another embodiment only the specificity determining residues from each of the CDRs described herein above are transferred to the human antibody framework.

When the CDRs or specificity determining residues are grafted, any appropriate acceptor variable region framework sequence may be used having regard to the class/type of the donor antibody from which the CDRs are derived, including mouse, primate and human framework regions. Suitably, the CDR-grafted antibody according to the present invention has a variable domain comprising human acceptor framework regions as well as one or more of the CDRs or specificity determining residues described above. Thus, provided in one embodiment is a neutralising CDR-grafted antibody wherein the variable domain comprises human acceptor framework regions and non-human donor CDRs.

Examples of human frameworks which can be used in the present invention are KOL, NEWM, REI, EU, TUR, TEI, LAY and POM (Kabat et al., supra). For example, KOL and NEWM can be used for the heavy chain, REI can be used for the light chain and EU, LAY and POM can be used for both the heavy chain and the light chain. Alternatively, human germline sequences may be used; these are available for example at: (see Worldwide Website: vbase2.org/) (see Retter et al, Nucl. Acids Res. (2005) 33 (supplement 1), D671-D674).

In a CDR-grafted antibody of the present invention, the acceptor heavy and light chains do not necessarily need to be derived from the same antibody and may, if desired, comprise composite chains having framework regions derived from different chains.

Also, in a CDR-grafted antibody of the present invention, the framework regions need not have exactly the same sequence as those of the acceptor antibody. For instance, unusual residues may be changed to more frequently occurring residues for that acceptor chain class or type. Alternatively, selected residues in the acceptor framework regions may be changed so that they correspond to the residue found at the same position in the donor antibody (see Reichmann et al., 1998, Nature, 332, 323-324). Such changes should be kept to the minimum necessary to recover the affinity of the donor antibody. A protocol for selecting residues in the acceptor framework regions which may need to be changed is set forth in WO 91/09967.

It will also be understood by one skilled in the art that antibodies may undergo a variety of posttranslational modifications. The type and extent of these modifications often depends on the host cell line used to express the antibody as well as the culture conditions. Such modifications may include variations in glycosylation, methionine oxidation, diketopiperazine formation, aspartate isomerization and asparagine deamidation. A frequent modification is the loss of a carboxy-terminal basic residue (such as lysine or arginine) due to the action of carboxypeptidases (as described in Harris, R J. Journal of Chromatography 705:129-134, 1995).

In one embodiment the antibody heavy chain comprises a CHi domain and the antibody light chain comprises a CL domain, either kappa or lambda.

Biological molecules, such as antibodies or fragments, contain acidic and/or basic functional groups, thereby giving the molecule a net positive or negative charge. The amount of overall “observed” charge will depend on the absolute amino acid sequence of the entity, the local environment of the charged groups in the 3D structure and the environmental conditions of the molecule. The isoelectric point (pI) is the pH at which a particular molecule or surface carries no net electrical charge. In one embodiment the antibody or fragment according to the present disclosure has an isoelectric point (pI) of at least 7. In one embodiment the antibody or fragment has an isoelectric point of at least 8, such as 8.5, 8.6, 8.7, 8.8 or 9. In one embodiment the pI of the antibody is 8. Programs such as ** ExPASY (see Worldwide Website: expasy.ch/tools/pi_tool.html) (see Walker, The Proteomics Protocols Handbook, Humana Press (2005), 571-607) may be used to predict the isoelectric point of the antibody or fragment.

Antibodies which bind to the epitope disclosed herein may comprise at least one, at least two or all three heavy chain CDR sequences of SEQ ID NOS: 4 to 6 (HCDR1/HCDR2/HCDR3 respectively). These are the HCDR1/HCDR2/HCDR3 sequences of the Ab 7326 antibody of the Examples as determined using Kabat methodology.

The Kabat and Chothia methods for determining CDR sequences are well known in the art (as well as other techniques). CDR sequences may be determined using any appropriate method and in the present invention, whilst Kabat is typically employed, other techniques could be used as well. In the present instance, SEQ ID NO: 3 presents the Ab 7326 HCDR1 sequence as determined using a combined Chothia & Kabat definition.

Antibodies of the invention may comprise at least one, at least two or all three light chain CDR sequences of SEQ ID NOS: 7 to 9 (LCDR1/LCDR2/LCDR3 respectively). These are the LCDR1/LCDR2/LCDR3 sequences of Ab 7326 using Kabat methodology.

The antibody preferably comprises at least a HCDR3 sequence of SEQ ID NO: 6.

Typically, the antibody comprises at least one heavy chain CDR sequence selected from SEQ ID NOS: 3 to 5 and at least one light chain CDR sequence selected from SEQ ID NOS 7 to 9. The antibody may comprise at least two heavy chain CDR sequences selected from SEQ ID NOS: 3 to 5 and at least two light chain CDR sequences selected from SEQ ID NOS: 7 to 9. The antibody typically comprises all three heavy chain CDR sequences of SEQ ID NOS: 3 to 5 (HCDR1/HCDR2/HCDR3 respectively) and all three light chain CDR sequences SEQ ID NOS: 7 to 9 (LCDR1/LCDR2/LCDR3 respectively). The antibodies may be chimeric, human or humanised antibodies.

The antibody may comprise a heavy chain variable region (HCVR) sequence of SEQ ID NO: 10 or 12 (the HCVR of Ab 7326 variants 1 and 2). The antibody may comprise a light chain variable region (LCVR) sequence of SEQ ID NO: 11 or 13 (the LCVR of Ab 7326 variants 1 and 2). The antibody preferably comprises the heavy chain variable region sequence of SEQ ID NO: 10 or 12 and the light chain variable region sequence of SEQ ID NO: 11 or 13 (especially HCVR/LVCR pairs of SEQ ID NOs: 10/11 or 12/13).

The antibody may comprise a heavy chain (H-chain) sequence of
SEQ ID NO: 14 mouse full length IgG1 heavy chain variant 1, or
SEQ ID NO: 28 mouse full length IgG1 heavy chain variant 2, or
SEQ ID NO: 30 human full length IgG1 heavy chain variant 1, or
SEQ ID NO: 16 human full length IgG1 heavy chain variant 2, or
SEQ ID NO: 22 human full length IgG4P heavy chain variant 1, or
SEQ ID NO: 34 human full-length IgG4P heavy chain variant 2, or
SEQ ID NO: 18 Fab heavy chain variant 1, or
SEQ ID NO: 32 Fab heavy chain variant 2.

The antibody may comprise a light chain (L-chain) sequence of
SEQ ID NO: 15 mouse full length IgG1 light chain variant 1, or
SEQ ID NO: 29 mouse full length IgG1 light chain variant 2, or
SEQ ID NO: 31 human full length IgG1 light chain variant 1, or
SEQ ID NO: 17 human full length IgG1 light chain variant 2, or
SEQ ID NO: 23 human full length IgG4P light chain variant 1, or
SEQ ID NO: 35 human full-length IgG4P light chain variant 2, or
SEQ ID NO: 19 Fab light chain variant 1, or
SEQ ID NO: 33 Fab light chain variant 2.

In one example, the antibody comprises a heavy chain/light chain sequence pair of
SEQ ID NOs: 14/15 mouse full length IgG1 variant 1, or
SEQ ID NOs: 28/29 mouse full length IgG1 variant 2, or
SEQ ID NOs: 30/31 human full length IgG1 variant 1, or
SEQ ID NOs: 16/17 human full length IgG1 variant 2, or
SEQ ID NOs: 22/23 human full length IgG4P variant 1, or
SEQ ID NOs: 34/35 human full-length IgG4P variant 2, or
SEQ ID NOs: 18/19 Fab light chain variant 1, or
SEQ ID NOs: 32/33 Fab light chain variant 2.

The variant forms of corresponding sequences may be interchanged. For example, the antibody may comprise a heavy chain/light chain sequence pair of
SEQ ID NOs: 14/29 mouse full length IgG1 heavy chain variant 1/light chain variant 2, or
SEQ ID NOs: 28/15 mouse full length IgG1 heavy chain variant 2/light chain variant 1, or
SEQ ID NOs: 30/17 human full length IgG1 heavy chain variant 1/light chain variant 2, or
SEQ ID NOs: 16/31 human full length IgG1 heavy chain variant 2/light chain variant 1, or
SEQ ID NOs: 22/35 human full length IgG4P heavy chain variant 1/light chain variant 2, or
SEQ ID NOs: 34/23 human full-length IgG4P heavy chain variant 2/light chain variant 1, or
SEQ ID NOs: 18/33 Fab light chain heavy chain variant 1/light chain variant 2, or
SEQ ID NOs: 32/19 Fab light chain heavy chain variant 2/light chain variant 1.

The antibodies may be chimeric, human or humanised antibodies.

The antibody may alternatively be or may comprise a variant of one of the specific sequences recited above. For example, a variant may be a substitution, deletion or addition variant of any of the above amino acid sequences.

A variant antibody may comprise 1, 2, 3, 4, 5, up to 10, up to 20 or more (typically up to a maximum of 50) amino acid substitutions and/or deletions from the specific sequences discussed above. “Deletion” variants may comprise the deletion of individual amino acids, deletion of small groups of amino acids such as 2, 3, 4 or 5 amino acids, or deletion of larger amino acid regions, such as the deletion of specific amino acid domains or other features. “Substitution” variants typically involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid or another aliphatic amino acid. Some properties of the 20 main amino acids which can be used to select suitable substituents are as follows:

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, neutral |
| Lys | polar, hydrophilic, charged(+) | Trp | aromatic, hydrophobic, neutral |
| Leu | aliphatic, hydrophobic, neutral | Tyr | aromatic, polar, hydrophobic |

“Derivatives” or “variants” generally include those in which instead of the naturally occurring amino acid the amino acid which appears in the sequence is a structural analog thereof. Amino acids used in the sequences may also be derivatized or modified, e.g. labelled, providing the function of the antibody is not significantly adversely affected.

Derivatives and variants as described above may be prepared during synthesis of the antibody or by post-production modification, or when the antibody is in recombinant form using the known techniques of site-directed mutagenesis, random mutagenesis, or enzymatic cleavage and/or ligation of nucleic acids.

Variant antibodies may have an amino acid sequence which has more than about 60%, or more than about 70%, e.g. 75 or 80%, typically more than about 85%, e.g. more than about 90 or 95% amino acid identity to the amino acid sequences disclosed herein (particularly the HCVR/LCVR sequences and the H- and L-chain sequences). Furthermore, the antibody may be a variant which has more than about 60%, or more than about 70%, e.g. 75 or 80%, typically more than about 85%, e.g. more than about 90 or 95% amino acid identity to the HCVR/LCVR sequences and the H- and L-chain sequences disclosed herein, whilst retaining the exact CDRs disclosed for these sequences. Variants may retain at least about 90%, 91%, 92%, 93%, 94%, 95%, 96%, 97%, 98% or 99% identity to the HCVR/LCVR sequences and to the H- and L-chain sequences disclosed herein (in some circumstances whilst retaining the exact CDRs).

Variants typically retain about 60%-about 99% identity, about 80%-about 99% identity, about 90%-about 99% identity or about 95%-about 99% identity. This level of amino acid identity may be seen across the full length of the relevant SEQ ID NO sequence or over a part of the sequence, such as across about 20, 30, 50, 75, 100, 150, 200 or more amino acids, depending on the size of the full length polypeptide.

In connection with amino acid sequences, “sequence identity” refers to sequences which have the stated value when assessed using ClustalW (Thompson et al., 1994, supra) with the following parameters:
Pairwise alignment parameters—Method: accurate, Matrix: PAM, Gap open penalty: 10.00, Gap extension penalty: 0.10;
Multiple alignment parameters—Matrix: PAM, Gap open penalty: 10.00, % identity for delay: 30, Penalize end gaps: on, Gap separation distance: 0, Negative matrix: no, Gap extension penalty: 0.20, Residue-specific gap penalties: on, Hydrophilic gap penalties: on, Hydrophilic residues: GPSNDQEKR. Sequence identity at a particular residue is intended to include identical residues which have simply been derivatized.

Antibodies having specific sequences and variants which maintain the function or activity of these chains are therefore provided.

Antibodies may compete for binding to Gremlin-1 with, or bind to the same epitope as, those defined above in terms of H-chain/L-chain, HCVR/LCVR or CDR sequences. In particular, an antibody may compete for binding to Gremlin-1 with, or bind to the same epitope as, an antibody which comprises a HCDR1/HCDR2/HCDR3/LCDR1/LCDR2/LCDR3 sequence combination of SEQ ID NOs: 4/5/6/7/8/9. An antibody may compete for binding to Gremlin-1 with, or bind to the same epitope as, an antibody which comprises a HCVR and LCVR sequence pair of SEQ ID NOs: 10/11 or 12/13 or full length chains of SEQ ID Nos: 14/15 or 16/17.

The term “epitope” is a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

One can easily determine whether an antibody binds to the same epitope as, or competes for binding with, a reference antibody by using routine methods known in the art. For example, to determine if a test antibody binds to the same epitope as a reference antibody of the invention, the reference antibody is allowed to bind to a protein or peptide under saturating conditions. Next, the ability of a test antibody to bind to the protein or peptide is assessed. If the test antibody is able to bind to the protein or peptide following saturation binding with the reference antibody, it can be concluded that the test antibody binds to a different epitope than the reference antibody. On the other hand, if the test antibody is not able to bind to protein or peptide following saturation binding with the reference antibody, then the test antibody may bind to the same epitope as the epitope bound by the reference antibody of the invention.

To determine if an antibody competes for binding with a reference antibody, the above-described binding methodology is performed in two orientations. In a first orientation, the reference antibody is allowed to bind to a protein/peptide under saturating conditions followed by assessment of binding of the test antibody to the protein/peptide molecule. In a second orientation, the test antibody is allowed to bind to the protein/peptide under saturating conditions followed by assessment of binding of the reference antibody to the protein/peptide. If, in both orientations, only the first (saturating) antibody is capable of binding to the protein/peptide, then it is concluded that the test antibody and the reference antibody compete for binding to the protein/peptide. As will be appreciated by the skilled person, an antibody that competes for binding with a reference antibody may not necessarily bind to the identical epitope as the reference antibody, but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50%, 75%, 90% or even 99% as measured in a competitive binding assay (see, e.g., Junghans et al., Cancer Res, 1990:50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

Antibodies can be tested for binding to Gremlin-1 by, for example, standard ELISA or Western blotting. An ELISA assay can also be used to screen for hybridomas that show positive reactivity with the target protein. The binding selectivity of an antibody may also be determined by monitoring binding of the antibody to cells expressing the target protein, for example by flow cytometry. Thus, a screening method may comprise the step of identifying an antibody that is capable of binding Gremlin-1 by carrying out an ELISA or Western blot or by flow cytometry.

Antibodies may selectively (or specifically) recognise Gremlin-1. An antibody, or other compound, “selectively binds” or “selectively recognises” a protein when it binds with preferential or high affinity to the protein for which it is selective but does not substantially bind, or binds with low affinity, to other proteins. The selectivity of an antibody may be further studied by determining whether or not the antibody binds to other related proteins as discussed above or whether it discriminates between them. Antibodies of the invention typically recognise human Gremlin-1.

Antibodies may also have cross-reactivity for related proteins, or for human Gremlin-1 and for Gremlin-1 from other species.

By specific (or selective), it will be understood that the antibody binds to the protein of interest with no significant cross-reactivity to any other molecule. Cross-reactivity may be assessed by any suitable method described herein. Cross-reactivity of an antibody may be considered significant if the antibody binds to the other molecule at least about 5%, 10%, 15%, 20%, 25%, 30%, 35%, 40%, 45%, 50%, 55%, 60%, 65%, 70%, 75%, 80%, 85%, 90% or 100% as strongly as it binds to the protein of interest. An antibody that is specific (or selective) may bind to another molecule at less than about 90%, 85%, 80%, 75%, 70%, 65%, 60%, 55%, 50%, 45%, 40%, 35%, 30%, 25% or 20% the strength that it binds to the protein of interest. The antibody may bind to the other molecule at less than about 20%, less than about 15%, less than about 10% or less than about 5%, less than about 2% or less than about 1% the strength that it binds to the protein of interest.

Anti-gremlin antibodies have been previously described, for example WO2014/159010A1 (Regeneron) describes anti-gremlin antibodies that inhibit Gremlin-1 activity, with binding affinity K_{D} values ranging from 625 pM to 270 nM at 25° C. Ciuclan et al (2013) describe an anti-Gremlin-1 monoclonal antibody with a binding affinity K_{D} 5.6×10⁻¹⁰ M.

The anti-Gremlin-1 antibodies of the invention are allosteric inhibitors of Gremlin-1 activity, and bind to a novel epitope, distal from the BMP binding site. The antibodies bind to Gremlin-1 with exceptionally high affinity with Kd values<100 pM. The antibodies of the invention therefore represent a significant improvement over currently available antibodies and are expected to be particularly useful for the treatment of Gremlin-1 mediated diseases.

Thus, antibodies suitable for use with the present invention may have a high affinity binding for (human) Gremlin-1. The antibody may have a dissociation constant (K_{D}) of less than <1 nM, and preferably <500 pM. In one example, the antibody has a dissociation constant (K_{D}) of less than 200 pM. In one example, the antibody has a dissociation constant (K_{D}) of less than 100 pM. A variety of methods can be used to determine the binding affinity of an antibody for its target antigen such as surface plasmon resonance assays, saturation assays, or immunoassays such as ELISA or RIA, as are well known to persons of skill in the art. An exemplary method for determining binding affinity is by surface plasmon resonance analysis on a BIAcore™ 2000 instrument (Biacore AB, Freiburg, Germany) using CM5 sensor chips, as described by Krinner et al., (2007) Mol. Immunol. February; 44 (5):916-25. (Epub 2006 May 11).

Antibodies of the invention are typically inhibitory antibodies. Gremlin-1 negatively regulates BMP-2, 4 and 7, so inhibition of Gremlin-1 results in increased signalling through BMP.

As mentioned above, the Examples of the present application describe two functional assays for screening whether an antibody is capable of inhibiting Gremlin 1, namely the SMAD phosphorylation assay and the Hek Id1 reporter gene assay. Typically, an inhibitory antibody restores SMAD phosphorylation and/or restores signalling of BMP in the Hek Id1 reporter gene assay. SMAD phosphorylation may be restored to at least 80%, 90% or 100% when compared with a BMP control. In the Hek Id1 reporter gene assay, an inhibitory antibody may have an IC₅₀ of less than 10 nM, preferably less than 5 nM.

Once a suitable antibody has been identified and selected, the amino acid sequence of the antibody may be identified by methods known in the art. The genes encoding the antibody can be cloned using degenerate primers. The antibody may be recombinantly produced by routine methods.

The present invention also provides an isolated DNA sequence encoding the heavy and/or light chain variable regions(s) (or the full length H- and L-chains) of an antibody molecule of the present invention.

A variant polynucleotide may comprise 1, 2, 3, 4, 5, up to 10, up to 20, up to 30, up to 40, up to 50, up to 75 or more nucleic acid substitutions and/or deletions from the sequences given in the sequence listing. Generally, a variant has 1-20, 1-50, 1-75 or 1-100 substitutions and/or deletions.

Suitable variants may be at least about 70% homologous to a polynucleotide of any one of nucleic acid sequences disclosed herein, typically at least about 80 or 90% and more suitably at least about 95%, 97% or 99% homologous thereto. Variants may retain at least about 90%, 91%, 92%, 93%, 94%, 95%, 96%, 97%, 98% or 99% identity. Variants typically retain about 60%-about 99% identity, about 80%-about 99% identity, about 90%-about 99% identity or about 95%-about 99% identity. Homology and identity at these levels is generally present at least with respect to the coding regions of the polynucleotides. Methods of measuring homology are well known in the art and it will be understood by those of skill in the art that in the present context, homology is calculated on the basis of nucleic acid identity. Such homology may exist over a region of at least about 15, at least about 30, for instance at least about 40, 60, 100, 200 or more contiguous nucleotides (depending on the length). Such homology may exist over the entire length of the unmodified polynucleotide sequence.

Methods of measuring polynucleotide homology or identity are known in the art. For example the UWGCG Package provides the BESTFIT program which can be used to calculate homology (e.g. used on its default settings) (Devereux et al (1984) Nucleic Acids Research 12, p 387-395).

The PILEUP and BLAST algorithms can also be used to calculate homology or line up sequences (typically on their default settings), for example as described in Altschul S. F. (1993) J Mol Evol 36:290-300; Altschul, S, F et al (1990) J Mol Biol 215:403-10.

Software for performing BLAST analysis is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89:10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90:5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, typically less than about 0.1, suitablyless than about 0.01, and most suitably less than about 0.001. For example, the smallest sum probability may be in the range of about 1-about 0.001, often about 0.01-about 0.001.

The homologue may differ from a sequence in the relevant polynucleotide by less than about 3, 5, 10, 15, 20 or more mutations (each of which may be a substitution, deletion or insertion). For example, the homologue may differ by 3-50 mutations, often 3-20 mutations. These mutations may be measured over a region of at least 30, for instance at least about 40, 60 or 100 or more contiguous nucleotides of the homologue.

In one embodiment, a variant sequence may vary from the specific sequences given in the sequence listing by virtue of the redundancy in the genetic code. The DNA code has 4 primary nucleic acid residues (A, T, C and G) and uses these to “spell” three letter codons which represent the amino acids the proteins encoded in an organism's genes. The linear sequence of codons along the DNA molecule is translated into the linear sequence of amino acids in the protein(s) encoded by those genes. The code is highly degenerate, with 61 codons coding for the 20 natural amino acids and 3 codons representing “stop” signals. Thus, most amino acids are coded for by more than one codon—in fact several are coded for by four or more different codons. A variant polynucleotide of the invention may therefore encode the same polypeptide sequence as another polynucleotide of the invention, but may have a different nucleic acid sequence due to the use of different codons to encode the same amino acids.

The DNA sequence of the present invention may comprise synthetic DNA, for instance produced by chemical processing, cDNA, genomic DNA or any combination thereof.

DNA sequences which encode an antibody molecule of the present invention can be obtained by methods well known to those skilled in the art. For example, DNA sequences coding for part or all of the antibody heavy and light chains may be synthesised as desired from the determined DNA sequences or on the basis of the corresponding amino acid sequences.

General methods by which the vectors may be constructed, transfection methods and culture methods are well known to those skilled in the art. In this respect, reference is made to “Current Protocols in Molecular Biology”, 1999, F. M. Ausubel (ed), Wiley Interscience, New York and the Maniatis Manual produced by Cold Spring Harbor Publishing.

Also provided is a host cell comprising one or more cloning or expression vectors comprising one or more DNA sequences encoding an antibody of the present invention. Any suitable host cell/vector system may be used for expression of the DNA sequences encoding the antibody molecule of the present invention. Bacterial, for example E. coli, and other microbial systems may be used or eukaryotic, for example mammalian, host cell expression systems may also be used. Suitable mammalian host cells include CHO, myeloma or hybridoma cells.

The present invention also provides a process for the production of an antibody molecule according to the present invention comprising culturing a host cell containing a vector of the present invention under conditions suitable for leading to expression of protein from DNA encoding the antibody molecule of the present invention, and isolating the antibody molecule.

The Ab 7326 antibody of the invention has been identified to bind the following residues of Gremlin-1: Ile110 (131), Lys126 (147), Lys127 (148), Phe128 (149), Thr129 (150), Thr130 (151), Arg148 (169), Lys153 (174) and Gln154 (175), where Lys126 (147), Lys127 (148), Phe128 (149), Thr129 (150), Thr130 (151), Arg148 (169), Lys153 (174) and Gln154 (175) are present on one Gremlin-1 monomer and Ile110 (131) is present on the second Gremlin-1 monomer. Numbering not in brackets is based on the structural file and (which matches the numbering of mouse Gremlin-2 based on structural alignment). The numbers in brackets represent the residues based on the UniProt entry 060565 of SEQ ID NO: 1. As discussed in the Examples section, these epitope residues were identified using NCONT analysis at 4 Å from the Gremlin-1-Ab 7326 Fab complex.

Antibodies of the invention may therefore bind to an epitope which comprises at least one residue selected from Ile131, Lys147, Lys148, Phe149, Thr150, Thr151, Arg169, Lys174 and Gln175 (with residue numbering based on SEQ ID NO: 1). Antibodies of the invention may bind an epitope which comprises 2, 3, 4, 5, 6, 7, 8 or all 9 of these residues (preferably at least 5 residues).

Antibodies of the invention may also recognise an epitope where Ile131 is present on a different Gremlin-1 monomer to the other residues.

Although these residues are provided for a particular sequence of human Gremlin-1, the skilled person could readily extrapolate the positions of these residues to other corresponding Gremlin sequences (e.g. mouse) using routine techniques. Antibodies binding to epitopes comprising the corresponding residues within these other Gremlin sequences are therefore also provided by the invention.

To screen for antibodies that bind to a particular epitope, a routine cross-blocking assay such as that described in Antibodies, Harlow and Lane (Cold Spring Harbor Press, Cold Spring Harb., NY) can be performed. Other methods include alanine scanning mutants, peptide blots (Reineke (2004) Methods Mol Biol 248:443-63), or peptide cleavage analysis. In addition, methods such as epitope excision, epitope extraction and chemical modification of antigens can be employed (Tomer (2000) Protein Science 9: 487-496). Such methods are well known in the art.

Antibody epitopes may also be determined by x-ray crystallography analysis. Antibodies of the present invention may therefore be assessed through x-ray crystallography analysis of the antibody bound to Gremlin-1. Epitopes may, in particular, be identified in this way by determining residues on Gremlin-1 within 4 Å of an antibody paratope residue.

### Pharmaceutical Compositions, Dosages and Dosage Regimes

An antibody of the invention, or an agent which modulates Gremlin-1 identified by the screening methods, may be provided in a pharmaceutical composition. The pharmaceutical composition will normally be sterile and will typically include a pharmaceutically acceptable carrier and/or adjuvant. A pharmaceutical composition of the present invention may additionally comprise a pharmaceutically acceptable adjuvant and/or carrier.

As used herein, “pharmaceutically acceptable carrier” includes any and all solvents, dispersion media, coatings, antibacterial and antifungal agents, isotonic and absorption delaying agents, and the like that are physiologically compatible. The carrier may be suitable for parenteral, e.g. intravenous, intramuscular, intradermal, intraocular, intraperitoneal, subcutaneous, spinal or other parenteral routes of administration, for example by injection or infusion. Alternatively, the carrier may be suitable for non-parenteral administration, such as a topical, epidermal or mucosal route of administration. The carrier may be suitable for oral administration. Depending on the route of administration, the modulator may be coated in a material to protect the compound from the action of acids and other natural conditions that may inactivate the compound.

The pharmaceutical compositions of the invention may include one or more pharmaceutically acceptable salts. A “pharmaceutically acceptable salt” refers to a salt that retains the desired biological activity of the parent compound and does not impart any undesired toxicological effects. Examples of such salts include acid addition salts and base addition salts.

Pharmaceutically acceptable carriers comprise aqueous carriers or diluents. Examples of suitable aqueous carriers that may be employed in the pharmaceutical compositions of the invention include water, buffered water and saline. Examples of other carriers include ethanol, polyols (such as glycerol, propylene glycol, polyethylene glycol, and the like), and suitable mixtures thereof, vegetable oils, such as olive oil, and injectable organic esters, such as ethyl oleate. In many cases, it will be desirable to include isotonic agents, for example, sugars, polyalcohols such as mannitol, sorbitol, or sodium chloride in the composition.

Therapeutic compositions typically must be sterile and stable under the conditions of manufacture and storage. The composition can be formulated as a solution, microemulsion, liposome, or other ordered structure suitable to high drug concentration.

Pharmaceutical compositions of the invention may comprise additional active ingredients.

Also within the scope of the present invention are kits comprising antibodies or modulatory agents of the invention and instructions for use. The kit may further contain one or more additional reagents, such as an additional therapeutic or prophylactic agent as discussed above.

The modulators and/or antibodies of the invention or formulations or compositions thereof may be administered for prophylactic and/or therapeutic treatments.

In therapeutic applications, compounds are administered to a subject already suffering from a disorder or condition as described above, in an amount sufficient to cure, alleviate or partially arrest the condition or one or more of its symptoms. Such therapeutic treatment may result in a decrease in severity of disease symptoms, or an increase in frequency or duration of symptom-free periods. An amount adequate to accomplish this is defined as a “therapeutically effective amount”.

In prophylactic applications, formulations are administered to a subject at risk of a disorder or condition as described above, in an amount sufficient to prevent or reduce the subsequent effects of the condition or one or more of its symptoms. An amount adequate to accomplish this is defined as a “prophylactically effective amount”. Effective amounts for each purpose will depend on the severity of the disease or injury as well as the weight and general state of the subject.

A subject for administration may be a human or non-human animal. The term “non-human animal” includes all vertebrates, e.g., mammals and non-mammals, such as non-human primates, sheep, dogs, cats, horses, cows, chickens, amphibians, reptiles, etc. Administration to humans is typical.

An antibody/modulator or pharmaceutical composition of the invention may be administered via one or more routes of administration using one or more of a variety of methods known in the art. As will be appreciated by the skilled artisan, the route and/or mode of administration will vary depending upon the desired results. Examples of routes of administration for compounds or pharmaceutical compositions of the invention include intravenous, intramuscular, intradermal, intraocular, intraperitoneal, subcutaneous, spinal or other parenteral routes of administration, for example by injection or infusion. The phrase “parenteral administration” as used herein means modes of administration other than enteral and topical administration, usually by injection. Alternatively, antibody/modulatory agent or pharmaceutical composition of the invention can be administered via a non-parenteral route, such as a topical, epidermal or mucosal route of administration. The antibody/modulatory agent or pharmaceutical composition of the invention may be for oral administration.

A suitable dosage of an antibody/modulatory agent or pharmaceutical composition of the invention may be determined by a skilled medical practitioner. Actual dosage levels of the active ingredients in the pharmaceutical compositions of the present invention may be varied so as to obtain an amount of the active ingredient that is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A suitable dose may be, for example, in the range of from about 0.01p g/kg to about 1000 mg/kg body weight, typically from about 0.1 μg/kg to about 100 mg/kg body weight, of the patient to be treated. For example, a suitable dosage may be from about 1 μg/kg to about 10 mg/kg body weight per day or from about 10 μg/kg to about 5 mg/kg body weight per day.

Dosage regimens may be adjusted to provide the optimum desired response (e.g., a therapeutic response). For example, a single dose may be administered, several divided doses may be administered over time or the dose may be proportionally reduced or increased as indicated by the exigencies of the therapeutic situation. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Administration may be in single or multiple doses. Multiple doses may be administered via the same or different routes and to the same or different locations. Alternatively, doses can be via a sustained release formulation, in which case less frequent administration is required. Dosage and frequency may vary depending on the half-life of the antagonist in the patient and the duration of treatment desired.

As mentioned above, modulators/antibodies or pharmaceutical compositions of the invention may be co-administered with one or other more other therapeutic agents.

Combined administration of two or more agents may be achieved in a number of different ways. Both may be administered together in a single composition, or they may be administered in separate compositions as part of a combined therapy. For example, the one may be administered before, after or concurrently with the other.

### Therapeutic Indications

Antibodies of present invention, or modulatory agents identified by the screening methods of the invention may be used in treating, preventing or ameliorating any condition that associated with Gremlin-1 activity. For example, any condition which results in whole or in part from signalling through Gremlin-1. In other words, the invention relates to the treatment, prevention or amelioration of conditions mediated or influenced by Gremlin. Such conditions include fibrotic disease including renal fibrosis (e.g. diabetic nephropathy and chronic allograft nephropathy) and idiopathic pulmonary fibrosis, pulmonary arterial hypertension, angiongenesis and cancer (e.g. colorectal cancer).

The following Examples illustrate the invention.

### Example 1—Protein Expression, Purification, Refolding and Structure Determination

### Protein Expression and Inclusion Body Preparation

A truncated human Gremlin-1 coding sequence (SEQ ID NO: 20), optimised for expression in E. coli, was cloned into a modified pET32a vector (Merck Millipore) using BamHI/XhoI, generating a vector encoding the Gremlin sequence with an N-terminal 6His-TEV tag (pET-hGremlin1).

### Expressed Sequence:

MGSSHHHHHHSSGENLYFQGSAMPGEEVLESSQEALHVTERKYLKRDWCKTQPL KQTHiEEGCNSRTIINRFCYGQCNSFYIPRHIRKEEGSFQSCSFCKPKKFTTMMVTL NCPELQPPTKKKRVTRVKQCRCISIDLD; SEQ ID NO: 2 (with non-Gremlin residues of the 6His-TEV tag shown in italics). Sequence numbering based on UniProt 060565 & SEQ ID NO: 1.

The pET-hGremlin1 plasmid DNA was used to transform BL21(DE3) cells. A single ampicillin resistant colony was picked from a LB/Amp agar plate and used to inoculate a 100 ml starter culture of LB/Amp. After shaking (200 rpm) for 16 hr at 37° C., 25 ml of the starter culture was used to inoculate 500 mL of 2×TY/Amp media. The culture was shaken (250 rpm) at 37° C. until an OD₆₀₀ of 3 was achieved. Subsequently, the culture was supplemented with 20 mL of a MOPS+glycerol feed mix (1M MOPS pH 7.4, 40% glycerol, 0.5% MgSO₄, 0.42% MgCl₂), induced with 300 μM IPTG and further incubated at 17° C., 180 rpm for 16 hours. Cells were harvested in a centrifuge (4,000 g for 20 minutes at 4° C.).

Cell pellets were resuspended in Lysis Buffer (PBS pH 7.4, 0.35 mg/ml lysozyme, g/ml DNase and 3 mM MgCl₂) at 4° C. and the insoluble fraction was harvested by centrifugation at 3,500 g for 30 minutes at 4° C. Pelleted inclusion bodies were washed three times by resuspending in wash buffer (50 mM Tris, 500 mM NaCl, 0.5% Triton X-100, pH 8.0), followed by centrifugation at 21,000 g for 15 minutes. An additional two washes were performed using wash buffer without Triton X-100.

### Solubilisation

Inclusion bodies were resuspended in denaturing buffer (8 M Urea, 100 mM Tris, 1 mM EDTA, 10 mM Na₂S₄O₆ and 100 mM Na₂SO₃, pH 8.5), stirred for 16 hrs at room-temperature and clarified by centrifugation at 21,000 g for 15 minutes.

### Pre-Refolding Purification

The solubilized inclusion bodies were loaded onto a Sephacryl S-200 26/60 column (120 mL) equilibrated in 8 M Urea, 50 mM MES, 200 mM NaCl, 1 mM EDTA, pH 6.0. Fractions containing Gremlin-1 protein were diluted with 6 M Urea, 20 mM MES, pH 6.0 and loaded onto HiTrap SP HP cation exchange columns and eluted with a 1 M NaCl gradient over 10 column volumes (10 CVs). Fractions containing purified, denatured hGremlin-1 protein were pooled.

### Refolding

Denatured purified Gremlin-1 protein was added drop-wise to re-folding buffer (50 mM Tris, pH 8.5, 150 mM NaCl, 5 mM GSH and 5 mM GSSG, 0.5 mM Cysteine, 5 mM EDTA, 0.5 M Arginine) to a final concentration of 0.1 mg/ml and incubated at 4° C. with constant stirring for 5 days. After 5 days the Gremlin-1 protein was dialysed against 20 mM HEPES, 100 mM NaCl, pH 7.5.

Following dialysis protein was applied to heparin HiTrap column and eluted using a gradient of 0-100% heparin elution buffer (20 mM HEPES, 1 M NaCl, pH 7.5) over 20 CV. Correctly folded protein eluted at 1 M NaCl whereas any misfolded protein eluted at lower salt concentrations.

Protein eluting at 1 M NaCl was concentrated and purified further on a S75 26/60 column equilibrated with 20 mM Hepes, pH 7.5, 1 M NaCl.

Protein was characterised by SDS PAGE (shift in gel), demonstrated to have the expected molecular weight and correct arrangement of disulphide bonds using liquid chromatography mass spectrometry (LC-MS) and to be active in a cell assay (IDl reporter assay).

### Gremlin 1 Structure Determination

Gremlin 1 protein crystals were grown using the hanging-drop method by mixing a solution of Gremlin 1 at 6.6 mg/ml and 0.1 M citric acid at pH 4, 1 M lithium chloride and 27% polyethylene glycol (PEG) 6000 in a 1:1 ratio. Before data collection, crystals were cryo-protected by adding 20% glycerol to the crystallization buffer. Diffraction data were collected at the Diamond Light Source and were processed using XDS (Kabsch, Wolfgang (2010) Acta Crystallographica Section D 66, 125-132). Diffraction data statistics are summarized in the table below:

TABLE 2
Diffraction data statistics

| Diffraction Statistics | | |
|---|---|---|
| Wavelength (Å) | 0.97949 | |
| Space group | C2 | |
| Cell dimensions | a = 84.55 Å, b = 107.22 Å, c = 77.09 Å; | |
| | α = 90.00°, β = 120.43°, γ = 90.00° | |
| Resolution range* (Å) | 26.19-2.72 | (2.79-2.72) |
| Completeness (%) | 98.5 | (99.0) |
| Multiplicity | 3.4 | (3.4) |
| I/sigma | 9.6 | (2.0) |
| Rmerge | 0.095 | (0.622) |
| Refinement Statistics | | |

| | |
|---|---|
| Resolution Range (Å) | 26.19-2.72 |
| R_{cryst} | 0.24 |
| R_{free} | 0.29 |
| R.m.s.d. bonds (Å)** | 0.013 |
| R.m.s.d. angles (°) | 1.782 |

*values in parenthesis correspond to the highest resolution shell
**r.m.s.d root mean square deviation

Gremlin-1 structure was solved by molecular replacement using Phaser (McCoy et al, J Appl Cryst (2007), 40, 658-674) and a Gremlin-1 model available from proprietary Gremlin-1/Fab complex coordinates. The resultant model of Gremlin-1 contained four copies of Gremlin 1 monomer organised as two dimers. Model corrections were made with Coot (Emsley et al Acta Crystallographica Section D: Biological Crystallography 66 (4), 486-501) and coordinates were refined using Refmac (Murshudov et al REFMAC5 for the refinement of macromolecular crystal structures. Acta Crystallographica Section D: Biological Crystallography. 2011; 67(Pt 4):355-367). Final coordinates were validated with Molprobity (Chen et al. (2010) MolProbity: all-atom structure validation for macromolecular crystallography. Acta Crystallographica D66:12-21). A summary of model refinement statistics is shown in Table 2 above.

### Example 2—BMP Binding Residues on Gremlin-1

As discussed above, Gremlin-1 belongs to the bone morphogenic protein (BMP) antagonist protein family within a sub-group known as the DAN family. Within the DAN family, Gremlin-1 shares greatest homology with Gremlin-2 (PRDC).

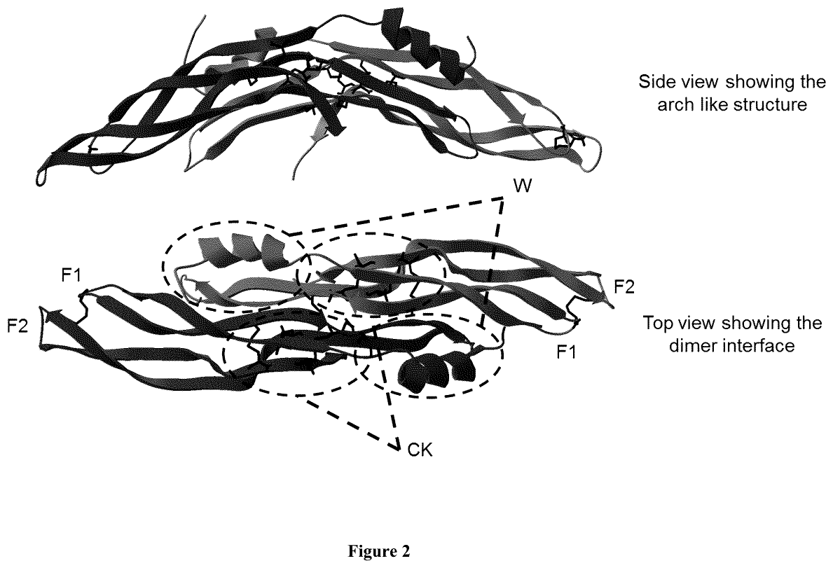

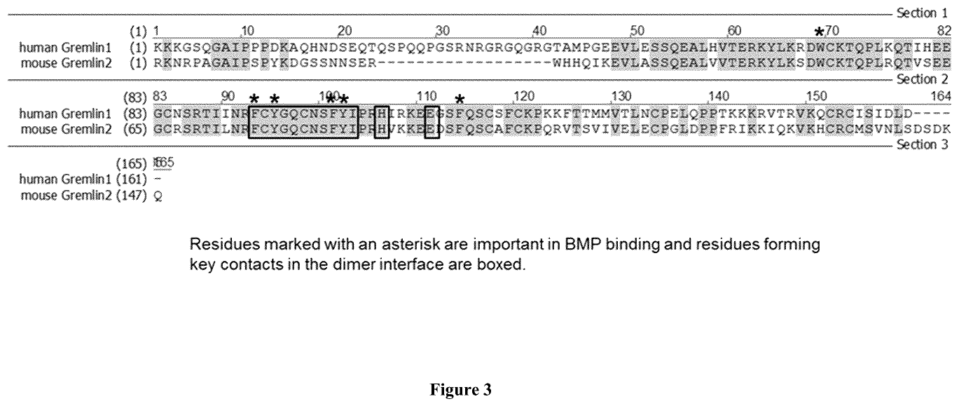

The 2.7 Å human Gremlin-1 structure described in Example 1 shares many features in common with the published mouse Gremlin-2 structure (Nolan et al (2013), Structure, 21, 1417-1429). The overall fold is very similar, with two copies of Gremlin-1 forming an antiparallel, non-covalent dimer, arranged in an arch. Each monomer adopts the characteristic finger-wrist-finger arrangement with a cystine-knot motif towards the wrist end, opposite the fingers (FIG. 2). Sequence identity between the proteins is 52% rising to 67% within the sequence visible in the two structures. The most highly conserved region lies in the extensive dimer interface where all the key contact residues are 100% conserved (FIG. 3).

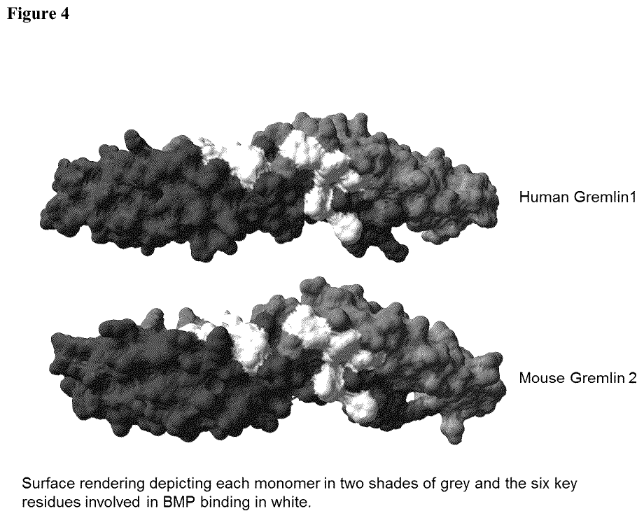

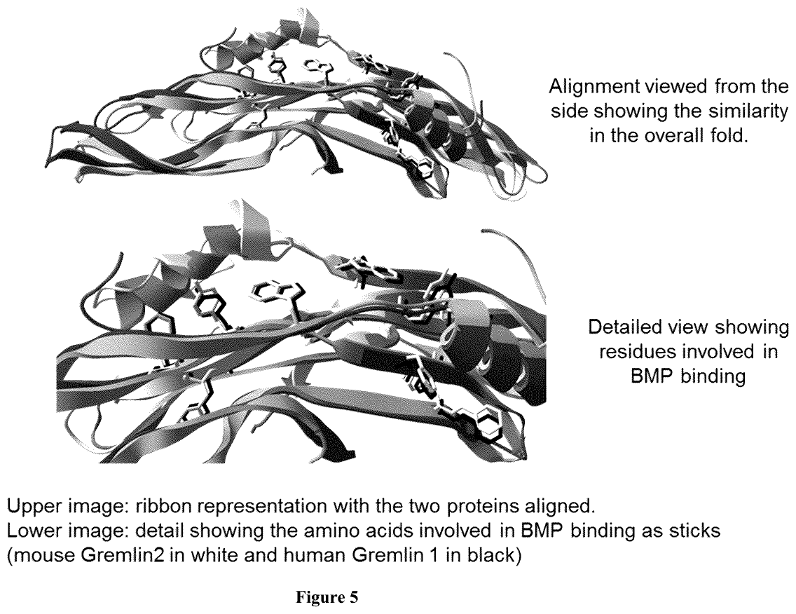

Residues involved in BMP's 2, 4 & 7 binding to mouse Gremlin-2 (PRDC) and DAN (NBL1) have been identified using mutagenesis (Nolan et al (2013), Structure, 21, 1417-1429 and Nolan et al (2014) J. Biol. Chem. 290, 4759-4771). The predicted BMP binding epitope encompasses a hydrophobic patch spanning across both monomers on the convex surface of the dimer (FIGS. 4 and 5). Six residues were identified by mutagenesis; Trp72, Phe96, Tyr98, Phe104, Tyr105 & Phe117 and are 100% conserved in human Gremlin-1 (numbering based on the mouse Gremlin-2 sequence). The degree of homology extends to the positioning of the side chains which adopt an identical conformation in both proteins (FIG. 5).

The amino acid numbering used in the Gremlin PDB file matches the numbering in the published mouse Gremlin-2 structure based on a structural alignment. This enables like for like comparison of amino acids when describing the structures. However, for clarity the key residues identified as playing a role in BMP binding are shown below with numbering based on the PDB file and UniProt file of SEQ ID NO: 1 in brackets: Trp72(93), Phe96(117), Tyr98(119), Phe104(125), Tyr105(126) & Phe117(138).

In both mouse Gremlin-2 and human Gremlin-1 the hydrophobic BMP binding epitope is partially buried by an alpha helix formed by the N-terminal residues of each protein. A model of BMP binding has been proposed whereby the N-terminus can flex, exposing the full BMP binding interface (Nolan et al (2013), Structure, 21, 1417-1429). In FIG. 4, the N-terminal residues have been removed from the human Gremlin-1 and mouse Gremlin-2 structures before rendering a surface to reveal the similarity of the BMP binding faces on each protein.

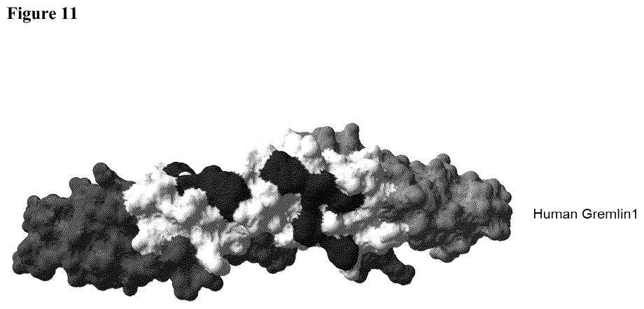

The literature only describes mutagenesis of six resides that have an effect on BMP binding. It is possible that the actual BMP epitope covers a larger surface area, encompassing neighbouring amino acids. By highlighting all residues, within 6 Å of those mutated, on the surface of Gremlin-1, a larger region of Gremlin-1 is revealed that could potentially be targeted by a therapeutic (FIG. 11). This more extensive region encompasses the following amino acids of human Gremlin-1:
Asp92-Leu99
Arg116-His130
Ser137-Ser142
Cys176-Cys178
(Numbering based on SEQ ID NO: 1)

By combining published information with the crystal structure information of human Gremlin-1, regions of human Gremlin-1 that offer themselves as a potential route for therapeutic intervention blocking its interaction with BMP's have been identified.

### Example 3—Hek Id1 Reporter Gene Assay

### Background

The Hek Id1 reporter gene assay uses Clone 12 Hek293-Id1 reporter cells. This cell line was stably transfected with Id1 transcription factor. Id1 is a transcription factor in the BMP signalling pathway. Gremlin is known to bind BMPs prevent binding to their receptors reducing the luciferase signal from the reporter gene. Therefore, using this reporter assay, it is possible to screen anti-Gremlin antibodies and see if there are any that block the interaction of Gremlin with BMPs. A restoration of the luciferase signal is seen in these cells if there is a blocking of this interaction.

### Method

Clone 12 cells were cultured in DMEM containing 10% FCS, 1×L-Glutamine & 1×NEAA. Cells are also grown in the presence of Hygromycin B (200 μg/ml) to ensure cells do not lose Id1 gene expression. Cells were assayed in DMEM containing 0.5% FCS, 1×L-Glutamine & 1× NEAA. Hygromycin B is not needed for the short time that the cells are in the assay.

The cells were washed in PBS, lifted off using cell dissociation buffer, spun and counted before being seeded at 5×104/well in 70 μl (Density of 7.14×10⁵/ml). Plates used were white, opaque Poly-D-Lysine coated 96-well sterile. Cells go in incubator for about 3-4 hours to settle down. BMP heterodimers were reconstituted to 200 μg/ml in 4 mM HCL. BMP was diluted to 10 μg/ml in assay media using a glass vial to give a new working stock.

In a polypropylene plate, Gremlin-1 was diluted 1:2 for an 8 point dose response curve with a top final dose of 1 μg/ml.

An additional volume of 20 μl media was added per well and plates were incubated at 37° C. for 45 mins.

BMP prepared at 100× was added to all wells except wells containing cells only. All wells are made up to 60 μl with assay medium and incubated for a further 45 mins at 37° C.

Post incubation, 30 μl of sample was transferred per well of assay plate and incubated for 20-24 hours before measuring luminescence signal.

Cell Steady Glo was thawed in advance at room temperature. Assay plates were cooled to room temperature for about 10-15 mins before adding the reagent. Luciferase signal was detected by addition of cell steady glo reagent (100 μl) for 20 minutes on shaker at room temperature and measuring luminescence using cell titre glo protocol on Synergy 2.

The maximum signal was generated from wells containing BMP and the minimum signal was generated from the wells containing cells only.

### Results

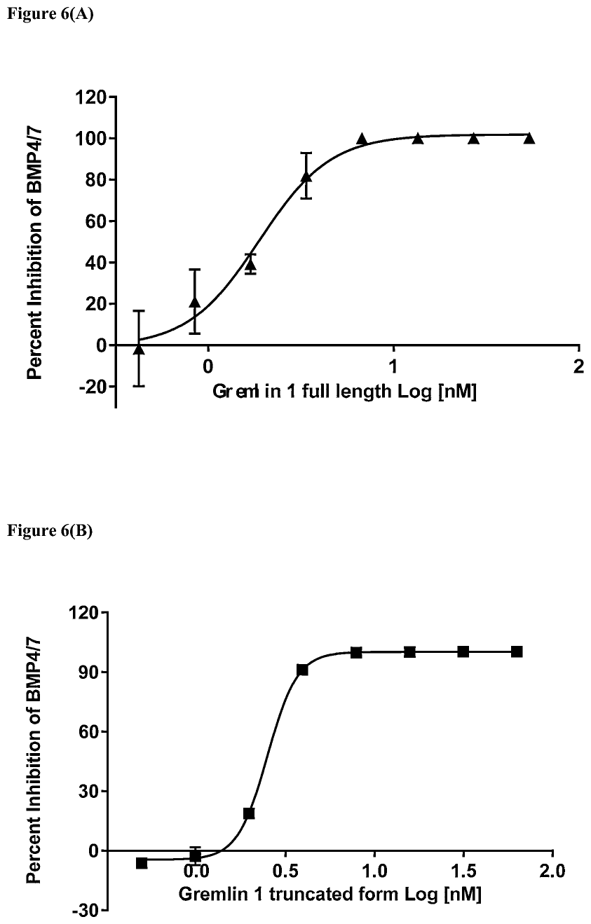

Gremlin-1 full length and truncated forms were tested in the Hek-Id1 reporter gene assay to confirm the blocking activity against BMP4/7. Results for full length protein are shown in FIG. 6A and results for truncated protein are shown in FIG. 6B.

The percentage of inhibition from dose response assays was calculated based on the maximum and minimum signals in the assay and the data fitted using 4 parameter logistical fit. The IC₅₀ was calculated based on the inflexion point of the curve.

TABLE 3
Potency results for full length Gremlin-1 and truncated
Gremlin-1 in the Hek-Id1 reporter gene assay.

| Hek-Id1 Reporter | | Geometric | 95% CI (or range |
|---|---|---|---|
| gene assay | N | mean (nM) | where N = <4) |
| Gremlin 1 Full | 2 | 1.6 | 1.3-1.9 |
| length | | | |
| Gremlin 1 truncated | 2 | 1.7 | 1.1-2.5 |

### Conclusion

Gremlin 1 was able to inhibit the BMP 4/7 signalling in the Hek-Id1 reporter gene assay.

### Example 4—Production of Anti-Gremlin-1 Antibodies

Anti-Gremlin-1 antibodies were derived by immunisation and library panning. The library was generated in-house as a naïve human library with the V-regions amplified from blood donations.

Immunisation yielded 26 distinct antibodies binding Gremlin-1 from the first round of immunisation. These antibodies were scaled up and purified for testing in screening assays.

25 human and mouse cross-reactive antibodies from the library were panned using recombinant human Gremlin from R&D Systems. 10 antibodies were selected for scale up and purified as scFvs for testing in the screening assays.

### Example 5—Screening of Anti-Gremlin-1 Antibodies

Antibodies were screened using the Hek-Id1 reporter gene assay described in Example 3 and by measuring SMAD phosphorylation. SMAD1, 5 and 8 are phosphorylated upon BMP signalling. Inhibitors of Gremlin-1 therefore increase SMAD phosphorylation.

SMAD phosphorylation assays were conducted on A549 cells or on human lung fibroblasts. Phosphorylation levels were determined using MSD.

### Results

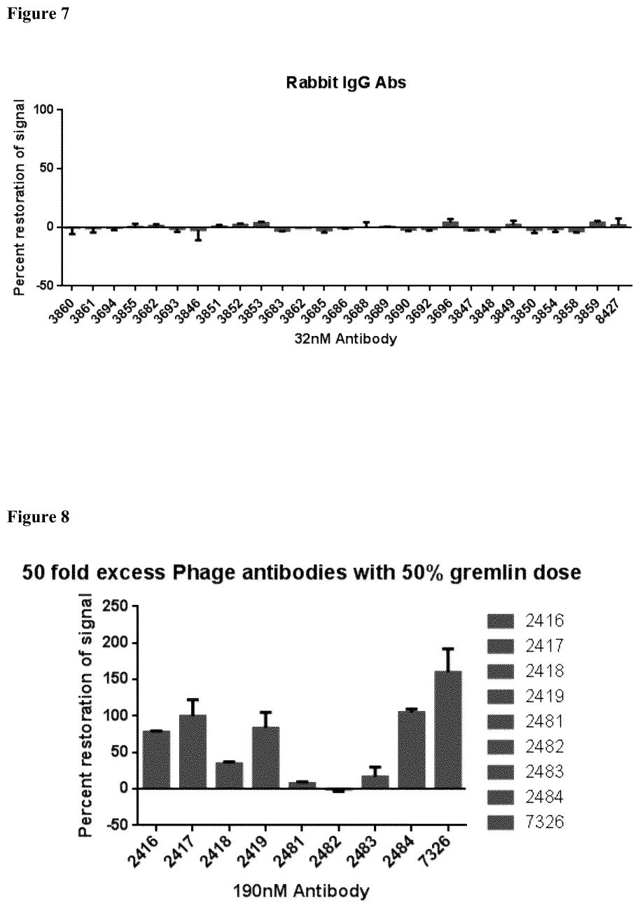

In the Hek-Id1 reporter gene assay, there were no apparent hits with the immunisation derived antibodies (with a 10 fold excess of antibody tested against a BMP4/7 heterodimer). Results are shown in FIG. 7.

In contrast, a number of library derived antibodies were capable of restoring signal in the Hek-Id1 reporter gene assay (50-fold excess of antibodies with a 50% gremlin dose) (FIG. 8). Of these, Ab2416 and Ab2417 contained high levels of endotoxin. Ab7326 maintained blocking ability at a 10-fold excess and 80% inhibition Gremlin-1 concentration.

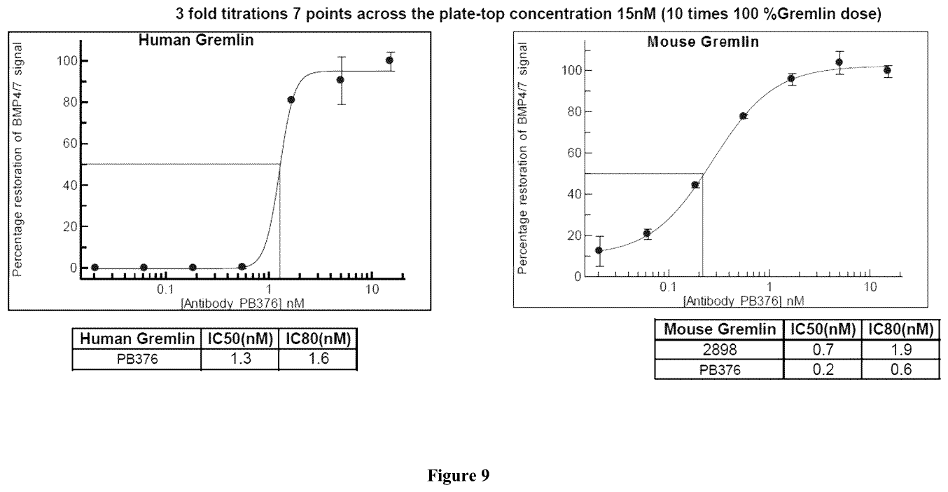

Additional results are presented in FIGS. 9A (human gremlin) and 9B (mouse Gremlin). These Figures show titrations of Ab7326 (labelled as PB376) up to 15 nM. Ab7326 was shown to restore signalling of BMP when blocked by either human (IC₅₀ of 1.3 nM) or mouse (IC₅₀ of 0.2 nM Gremlin). The antibody functions both as a human and mouse IgG1.

Sequences of the mouse and human full length IgG1 are presented below. In order to synthesise the mouse and human full length IgG1 proteins, the Ab7326 variable regions derived from the library were re-cloned into vectors comprising the appropriate antibody constant domains.

Because Ab7326 came from a naïve human library, where Abs are cloned as scFvs, in order to re-clone the 7326 variable regions as full length Abs or Fabs, it was necessary to PCR amplify the VH and VK using pools of primers/degenerate primers. The amplified PCR products were then digested and cloned simultaneously into mouse and human vectors. As the VH and VK were amplified by pools of primers/degenerate primers, two variant forms of the products were obtained, differing by a single amino acid residue derived from subtly different primers annealing during the PCR process.

The two variant forms of heavy chain variable region differed by a single amino acid at position 6, and the two variant forms of the light chain variable region differed by a single amino acid at position 7, as shown below:
Heavy chain variable region variant 1 has glutamic acid (E) at position 6.
Heavy chain variable region variant 2 has glutamine (Q) at position 6.
Light chain variable region variant 1 has serine (S) at position 7.
Light chain variable region variant 2 has threonine (T) at position 7.

Mouse full length IgG1 - heavy chain variant 1
(SEQ ID NO: 14)
QVQLVESGAE VKKPGATVKI SCKVSGYTFT DYYMHWVQQA
PGKGLEWMGL VDPEDGETIY AEKFQGRVTI TADTSTDTAY
MELSSLRSED TAVYYCATDA RGSGSYYPNH FDYWGQGTLV
TVSSAKTTPP SVYPLAPGSA AQTNSMVTLG CLVKGYFPEP
VIVTWNSGSL SSGVHTFPAV LQSDLYTLSS SVTVPSSTWP
SETVTCNVAH PASSTKVDKK IVPRDCGCKP CICTVPEVSS
VFIFPPKPKD VLTITLTPKV TCVVVDISKD DPEVQFSWFV
DDVEVHTAQT QPREEQFNST FRSVSELPIM HQDWINGKEF
KCRVNSAAFP APIEKTISKT KGRPKAPQVY TIPPPKEQMA
KDKVSLTCMI TDFFPEDITV EWQWNGQPAE NYKNTQPIMD
TDGSYFVYSK LNVQKSNWEA GNTFTCSVLH EGLHNHHTEK
SLSHSPGK
Mouse full length IgG1 - light chain variant 1
(SEQ ID NO: 15)
DIVMIQSPDS LAVSLGERAT INCKSSQSVL YSSNNKNYLA
WYQQKPGQPP KLLIYWASTR ESGVPDRFSG SGSGTDFTLT
INSLQAEDVA VYFCQQYYDT PTFGQGTRLE IKRTDAAPTV
SIFPPSSEQL TSGGASVVCF LNNFYPKDIN VKWKIDGSER
QNGVINSWTD QDSKDSTYSM SSTLTLTKDE YERHNSYTCE
ATHKTSTSPI VKSFNRNEC
Mouse full length IgG1 - heavy chain variant 2
(SEQ ID NO: 28)
QVQLVQSGAE VKKPGATVKI SCKVSGYTFT DYYMHWVQQA
PGKGLEWMGL VDPEDGETIY AEKFQGRVTI TADTSTDTAY
MELSSLRSED TAVYYCATDA RGSGSYYPNH FDYWGQGTLV
TVSSAKTTPP SVYPLAPGSA AQTNSMVTLG CLVKGYFPEP
VTVTWNSGSL SSGVHTFPAV LQSDLYTLSS SVTVPSSTWP
SETVTCNVAH PASSTKVDKK IVPRDCGCKP CICTVPEVSS
VFIFPPKPKD VLTITLTPKV TCVVVDISKD DPEVQFSWFV
DDVEVHTAQT QPREEQFNST FRSVSELPIM HQDWINGKEF
KCRVNSAAFP APIEKTISKT KGRPKAPQVY TIPPPKEQMA
KDKVSLTCMI TDFFPEDITV EWQWNGQPAE NYKNTQPIMD
TDGSYFVYSK LNVQKSNWEA GNTFTCSVLH EGLHNHHTEK
SLSHSPGK
Mouse full length IgG1 - light chain variant 2
(SEQ ID NO: 29)
DIVMTQTPDS LAVSLGERAT INCKSSQSVL YSSNNKNYLA
WYQQKPGQPP KLLIYWASTR ESGVPDRFSG SGSGTDFTLT
INSLQAEDVA VYFCQQYYDT PTFGQGTRLE IKRTDAAPTV
SIFPPSSEQL TSGGASVVCF LNNFYPKDIN VKWKIDGSER
QNGVLNSWTD QDSKDSTYSM SSTLTLTKDE YERHNSYTCE
ATHKTSTSPI VKSFNRNEC
Human full length IgG1 - heavy chain variant 1
(SEQ ID NO: 30)
QVQLVESGAE VKKPGATVKI SCKVSGYTFT DYYMHWVQQA
PGKGLEWMGL VDPEDGETIY AEKFQGRVTI TADTSTDTAY
MELSSLRSED TAVYYCATDA RGSGSYYPNH FDYWGQGTLV
TVSSASTKGP SVFPLAPSSK STSGGTAALG CLVKDYFPEP
VTVSWNSGAL TSGVHTFPAV LQSSGLYSLS SVVTVPSSSL
GTQTYICNVN HKPSNTKVDK KVEPKSCDKT HTCPPCPAPE
LLGGPSVFLF PPKPKDTLMI SRTPEVTCVV VDVSHEDPEV
KFNWYVDGVE VHNAKTKPRE EQYNSTYRVV SVLTVLHQDW
LNGKEYKCKV SNKALPAPIE KTISKAKGQP REPQVYTLPP
SRDELTKNQV SLTCLVKGFY PSDIAVEWES NGQPENNYKT
TPPVLDSDGS FFLYSKLTVD KSRWQQGNVF SCSVMHEALH
NHYTQKSLSL SPGK
Human full length IgG1 - light chain variant 1
(SEQ ID NO: 31)
DIVMTQSPDS LAVSLGERAT INCKSSQSVL YSSNNKNYLA
WYQQKPGQPP KLLIYWASTR ESGVPDRFSG SGSGTDFTLT
INSLQAEDVA VYFCQQYYDT PTFGQGTRLE IKRTVAAPSV
FIFPPSDEQL KSGTASVVCL LNNFYPREAK VQWKVDNALQ
SGNSQESVTE QDSKDSTYSL SSTLTLSKAD YEKHKVYACE
VTHQGLSSPV TKSFNRGEC
Human full length IgG1 - heavy chain variant 2
(SEQ ID NO: 16)
QVQLVQSGAE VKKPGATVKI SCKVSGYTFT DYYMHWVQQA
PGKGLEWMGL VDPEDGETIY AEKFQGRVTI TADTSTDTAY
MELSSLRSED TAVYYCATDA RGSGSYYPNH FDYWGQGTLV
TVSSASTKGP SVFPLAPSSK STSGGTAALG CLVKDYFPEP
VTVSWNSGAL TSGVHTFPAV LQSSGLYSLS SVVTVPSSSL
GTQTYICNVN HKPSNTKVDK KVEPKSCDKT HTCPPCPAPE
LLGGPSVFLF PPKPKDTIMI SRTPEVTCVV VDVSHEDPEV
KFNWYVDGVE VHNAKTKPRE EQYNSTYRVV SVLTVLHQDW
LNGKEYKCKV SNKALPAPIE KTISKAKGQP REPQVYTLPP
SRDELTKNQV SLTCLVKGFY PSDIAVEWES NGQPENNYKT
TPPVLDSDGS FFLYSKLTVD KSRWQQGNVF SCSVMHEALH
NHYTQKSLSL SPGK
Human full length IgG1 - light chain variant 2
(SEQ ID NO: 17)
DIVMTQTPDS LAVSLGERAT INCKSSQSVL YSSNNKNYLA
WYQQKPGQPP KLLIYWASTR ESGVPDRFSG SGSGTDFTLT
INSLQAEDVA VYFCQQYYDT PTFGQGTRLE IKRTVAAPSV
FIFPPSDEQL KSGTASVVCL LNNFYPREAK VQWKVDNALQ
SGNSQESVTE QDSKDSTYSL SSTLTLSKAD YEKHKVYACE
VTHQGLSSPV TKSFNRGEC

Antibody CDRs were determined using the Kabat method (highlighted in bold in the above sequences) Additional HCDR1 residues using the Chothia definition are in italics. Constant region sequences are also underlined.

Restoration of p-SMAD signalling with anti-Gremlin 1 antibodies is shown in Table 4 below.

TABLE 4
Restoration of p-SMAD signalling

| | 2417 | 2418 | 2419 | 2481 | 2482 | 2483 | 2484 | 7326 | 8427 |
|---|---|---|---|---|---|---|---|---|---|
| BMP 2 | 109.1% +/− | 58.2% +/− | 32.6% +/− | 40.4% +/− | 35.3% +/− | 43.1% +/− | 104.0% +/− | 107.2% +/− | 51.3% +/− |
| 50 ng/ml | 2.8% | 1.9% | 1.4% | 0.6% | 0.8% | 2.1% | 2.7% | 3.5% | 1.4% |
| BMP 4 | 109.6% +/− | 71.3% +/− | 31.7% +/− | 60.1% +/− | 54.4% +/− | 72.5% +/− | 105.2% +/− | 110.0% +/− | 78.2% +/− |
| 25 ng/ml | 3.0% | 3.1% | 1.2% | 2.2% | 1.3% | 2.1% | 3.3% | 3.8% | 2.5% |
| BMP 7 | 111.5% +/− | 99.5% +/− | 53.8% +/− | 64.4% +/− | 52.3% +/− | 66.2% +/− | 105.2% +/− | 108.0% +/− | 72.6% +/− |
| 200 ng/ml | 3.8% | 3.2% | 3.4% | 1.3% | 1.1% | 1.2% | 4.3% | 3.2% | 2.5% |
| BMP-2/7 | 119.3% +/− | 78.6% +/− | 50.8% +/− | 53.7% +/− | 47.6% +/− | 56.1% +/− | 120.4% +/− | 128.5% +/− | 62.8% +/− |
| 50 ng/ml | 2.6% | 3.6% | 2.7% | 3.1% | 1.5% | 2.5% | 4.4% | 2.9% | 2.5% |
| BMP4/7 | 113.7% +/− | 78.0% +/− | 61.4% +/− | 48.3% +/− | 41.7% +/− | 50.8% +/− | 112.4% +/− | 127.0% +/− | 63.3% +/− |
| 50 ng/ml | 3.1% | 4.0% | 4.0% | 2.1% | 1.7% | 1.7% | 2.5% | 3.1% | 2.1% |

Results are shown as a percentage of SMAD phosphorylation by BMP alone (control BMP). Experiments were performed using lung fibroblasts from idiopathic pulmonary fibrosis patients. rhGremlin-1 and the anti-Gremlin-1 antibodies were preincubated for 45 minutes at room temperature. rhGremlin-1 and the anti-Gremlin-1 antibodies were then added with BMP to the cells for 30 minutes.

Table 5 then shows further results in the SMAD phosphorylation assay, where displacement of BMP-2 or BMP4/7 from Gremlin 1-BMP complexes by anti-Gremlin-1 antibodies was investigated. Experiments were again performed using lung fibroblasts from idiopathic pulmonary fibrosis patients. rhBMP-2 or rhBMP 4/7 were preincubated with rhGremlin-1 for 1 hour at room temperature. The BMP-2- or BMP4/7-Gremlin-1 complexes were incubated with different concentrations of the anti-Gremlin-1 antibodies overnight at 4° C. Antibody concentrations represent the final concentration on the plate.

TABLE 5
Displacement of BMP-2 or BMP4/7 from Gremlin 1-BMP complexes by anti-Gremlin-1 antibodies

| | | 81.3 | 40.6 | 20.3 | 10.2 | 5.1 | 2.55 | 1.27 | 0.63 |
|---|---|---|---|---|---|---|---|---|---|
| | | μg/ml | μg/ml | μg/ml | μg/ml | μg/ml | μg/ml | μg/ml | μg/ml |
| 2484 | BMP 2 | 100.3% +/− | 98.8% +/− | 97.0% +/− | 93.5% +/− | 86.4% +/− | 79.9% +/− | 66.5% +/− | 54.8% +/− |
| | 50 ng/ml | 3.5% | 2.7% | 2.9% | 2.6% | 2.0% | 1.9% | 2.8% | 0.3% |
| 2484 | BMP4/7 | 136.4% +/− | 133.2% +/− | 121.4% +/− | 108.1% +/− | 86.6% +/− | 74.7% +/− | 65.8% +/− | 60.7% +/− |
| | 50 ng/ml | 4.2% | 1.0% | 1.4% | 4.9% | 4.4% | 2.2% | 0.6% | 1.5% |
| 7326 | BMP 2 | 103.7% +/− | 101.5% +/− | 99.4% +/− | 103.8% +/− | 100.3% +/− | 103.2% +/− | 102.8% +/− | 97.0% +/− |
| | 50 ng/ml | 1.1% | 2.4% | 3.8% | 2.4% | 2.2% | 4.3% | 2.8% | 2.9% |
| 7326 | BMP4/7 | 133.7% +/− | 132.3% +/− | 130.3% +/− | 125.6% +/− | 121.4% +/− | 120.9% +/− | 111.1% +/− | 102.0% +/− |
| | 50 ng/ml | 0.8% | 1.8% | 4.2% | 10.0% | 4.2% | 3.3% | 2.3% | 4.5% |

The results shown in Table 5 demonstrate that Ab7326 can displace already complexed BMP-2 or BMP4/7 from Gremlin 1-BMP complexes. Ab7326 can achieve this displacement at much lower concentrations that the comparison antibody 2484. This provides evidence that Ab7326 is an allosteric inhibitor, consistent with our finding that the binding site for Ab7326 is distal from the known BMP binding regions on gremlin-1. Thus Ab7326 is able to access the allosteric binding site even when BMP is complexed to gremlin-1, resulting in significantly improved inhibition of gremlin activity.

### Example 6—Obtaining the Crystal Structure of Gremlin-1 in Complex with the 7326 Fab

The crystal structure of human Gremlin-1 in complex with Ab 7326 Fab was solved at a resolution of 2.1 Å. Fab sequences are shown below:

Heavy chain:
SEQ ID NO: 18
QVQLVESGAEVKKPGATVKISCKVSGYTFTDYYMHWVQQAPGKGLEWMGL
VDPEDGETIYAEKFQGRVTITADTSTDTAYMELSSLRSEDTAVYYCATDA
RGSGSYYPNHFDYWGQGTLVTVSSASTKGPSVFPLAPSSKSTSGGTAALG
CLVKDYFPEPVTVSWNSGALTSGVHTFPAVLQSSGLYSLSSVVTVPSSSL
GTQTYICNVNHKPSNTKVDKKVEPKSC
Light chain:
SEQ ID NO: 19
DIVMTQSPDSLAVSLGERATINCKSSQSVLYSSNNKNYLAWYQQKPGQPP
KLLIYWASTRESGVPDRFSGSGSGTDFTLTINSLQAEDVAVYFCQQYYDT
PTFGQGTRLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

The CCP4 software NCONT was then used to identify all contacts at 4 Å between Gremlin-1 and the Fab. The following residues were identified: Ile131, Lys147, Lys148, Phe149, Thr150, Thr151, Arg169, Lys174 and Gln175 (numbering based on the UniProt Sequence of SEQ ID NO: 1 (numbered as Ile110, Lys126, Lys127, Phe128, Thr129, Thr130, Arg148, Lys153 and Gln154 in the structure file which matches the numbering of mouse Gremlin-2).

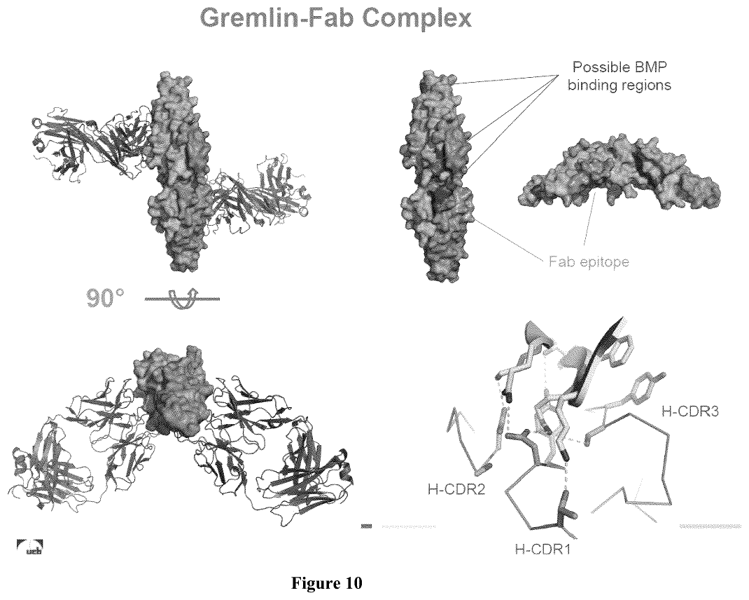

FIG. 10 shows structural models of the Gremlin-Fab complex, with the Fab epitope residues shown relative to the BMP binding regions.

Ab 7326 is an inhibitory antibody which acts allosterically, i.e. it binds away from the BMP binding regions.

### Example 7—Affinity Measurements for Binding of Anti-Gremlin-1 Antibody Ab7326 to Gremlin-1

### Method

The affinity of anti-Gremlin mIgG for human Gremlin 1 was determined by biamolecular interaction analysis using surface plasmon resonance (SPR) technology on a Biacore T200 system, GE Healthcare Bio-Sciences AB. Anti-Gremlin mIgG was captured by an immobilised anti-mouse Fc surface and Gremlin 1 was titrated over the captured mIgG.

The capture ligand (affinipure F(ab′)₂ fragment of goat anti-mouse IgG, Fc fragment specific, 115-006-071, Jackson ImmunoResearch Inc.) was immobilised at 50p g/ml in 10 mM NaAc, pH5.0 on flow cell 2 of a CM4 Sensor Chip via amine coupling chemistry, using 600s activation and deactivation injections, to a level of ˜1600 response units (RU). HBS-EP+ buffer (0.01 M HEPES pH 7.4, 0.15 M NaCl, 3 mM EDTA, 0.05% Surfactant P20) was used as the running buffer with a flow rate of 10 μl/min. A reference surface was prepared on flow cell 1 by activating and deactivating the surface as for flow cell 2 but omitting the capture ligand.

The assay buffer was HBS-EP+ plus an extra 150 mM NaCl to give a final NaCl concentration of 300 mM plus 1% CMD40. A 60s injection of anti-Gremlin mIgG (at 5 μg/ml in running buffer) was passed over flow cells 1 and 2 to give a capture level of approximately 100 RU on the immobilised anti-mouse IgG, Fc surface. Recombinant human Gremlin 1 was titrated in running buffer from 5 nM (using 2-fold dilutions) and injected over flow cells 1 and 2 at a flow rate of 30 μl/min for 3 min followed by a 5 min dissociation phase. A buffer only control was also included. The surface was regenerated at a flow rate of 10 μl/min by a 60s injection of 50 mM HCl, a 30s injection of 5 mM NaOH and a 30s injection of 50 mM HCl.

The kinetic data was determined using Biacore T200 evaluation software.

The affinity measurements were made at 25° C.

### Results

Binding affinity, taken as the average K_{D} value for 5 determinations, was found to be below 100 pM.

### Example 8—Assessment of In Vivo Effects of Anti-Gremlin-1 Antibodies on Chronic Hypoxia/SU5416-Induced Pulmonary Arterial Hypertension in Mice

### Summary

Imbalance in the TGFβ superfamily has been strongly implicated in a number of pulmonary pathologies, including pulmonary arterial hypertension (PAH) (Budd & Holmes Pharm Ther 2012). Gremlin-1 has been implicated in the development and progression of PAH (Thomas et al AJP 2009; Ciuclan et al AJP 2013). Recent studies have demonstrated that an anti-gremlin-1 antibody can inhibit the development of pulmonary arterial hypertension in the pre-clinical hypoxia/SU5416 model of PAH (Ciuclan et al AJP 2013). Here we assessed the effects of anti-Gremlini antibodies on hemodynamic and vascular remodeling in the pre-clinical hypoxia/SU5416 mouse model of PAH.

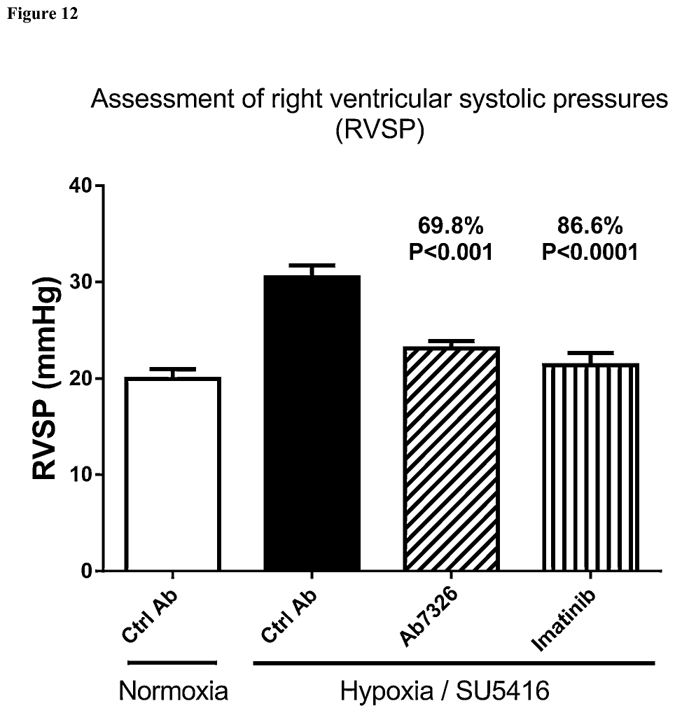

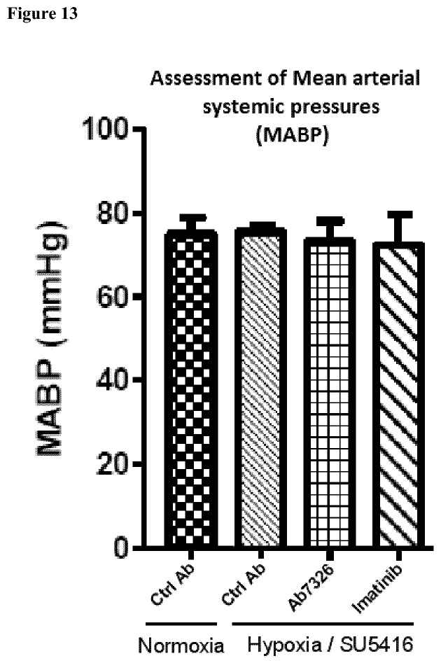

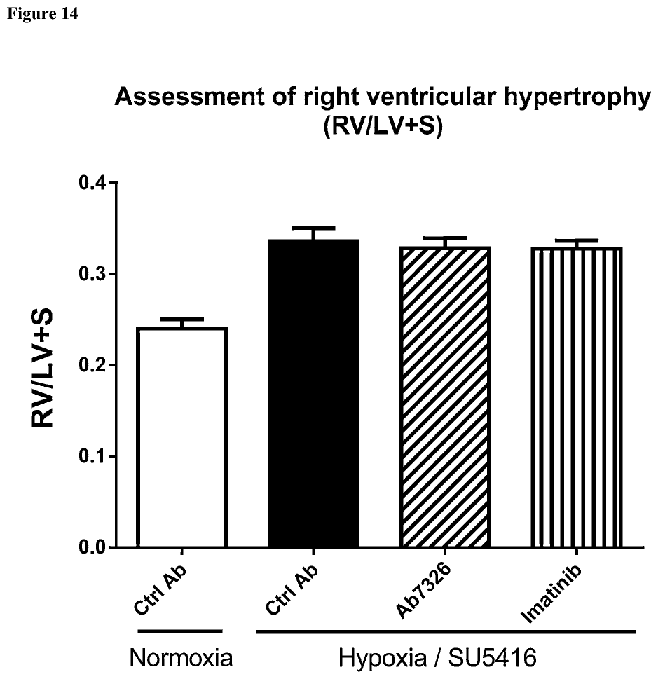

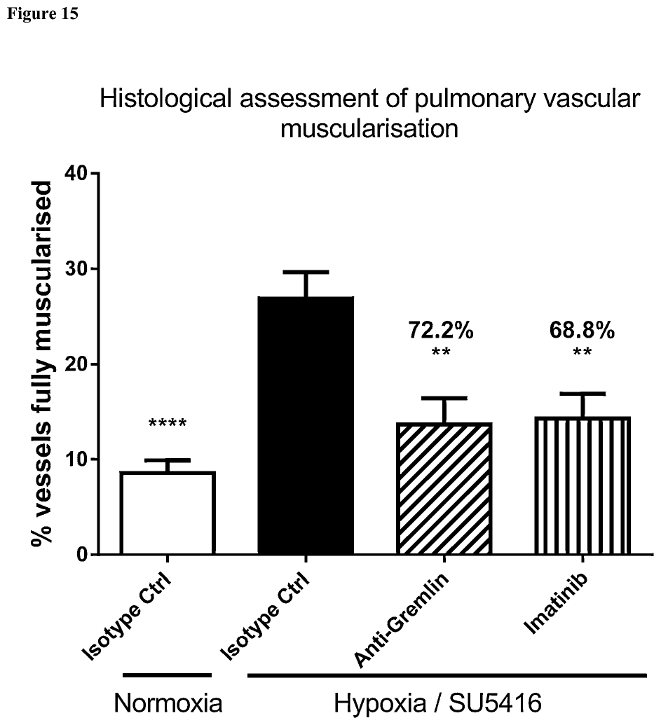

Hypoxia/SU5416 led to a significant increase in right ventricular systolic pressures (RVSP) and right heart hypertrophy (FIGS. 12 and 15). Administration of anti-Gremlin-1 led to a significant (P<0.005) reduction in RVSP compared to IgG1 and PBS control groups. No significant effect of anti-Gremlin-1 was observed on RVSP in animals maintained in normoxia (FIG. 12). No effect was observed on systemic pressures (mean arterial blood pressure or MABP; FIG. 13), or right heart hypertrophy (FIG. 14). Collectively this study supports the role of gremlin-1 in the development of pulmonary vascular remodelling and raised RVSPs in the chronic hypoxia/SU5416 model of PAH and the use of anti-gremlin-1 antibodies in its treatment.

### Background

Pulmonary hypertension (PH) is the hemodynamic state in which the pressure measured in the pulmonary artery is elevated. Clinically this is defined by a mean resting pulmonary arterial pressure (mPAP) that is >25 mmHg, pulmonary vascular resistance (PVR)>3 Wood units and pulmonary wedge pressure<15 mmHg (Badesch et al 2009). The pathological characteristics of PH are multifaceted and include pulmonary arterial pressure, vascular remodelling of the small to medium arteries, right ventricular hypertrophy and ultimately right heart failure (Faber & Loscalzo 2004). PH is classified by the WHO into five major categories, including group I. Group I represents pulmonary arterial hypertension which includes idiopathic PAH and heritable PAH as well as associated PAH which results in conjunction with other complications such as systemic sclerosis (Simonneau et al 2009). A number of pre-disposing mutations have been linked to the development of PAH in heritable and idiopathic PAH patients the most predominant being mutations to the bone morphogenetic protein receptor, BMPR2 (Budd & Holmes Pharm Ther 2012). In addition mutations in the BMP activated downstream signaling components Smads have also been reported in patients developing PAH (Budd & Holmes Pharm Ther 2012). More recently, studies have identified enhanced levels of the BMP inhibitor, gremlin (gremlin-1 and 2) in patients with PAH (Cahill et al Circ 2012). Consistent with a functional role for gremlin-1 in the development of PAH, haplodeficiency of gremlin-1 augmented BMP signaling in the hypoxic mouse lung and lead to reduced pulmonary vascular resistance by attenuating vascular remodeling (Cahill et al Circ 2012). These observations were further supported by Ciuclan and colleagues (AJP 2013) who demonstrated an anti-gremlin-1 antibody ameliorates chronic hypoxia/SU5416-induced pulmonary arterial hypertension in mice. Recent studies suggest non-genetic mechanisms may contribute to reduced BMPR2 expression in systemic sclerosis patients and may contribute to the development of PAH (Gilbane et al AJRCCM). Collectively, imbalance in the BMP superfamily axis may lead to the development of pulmonary pathologies such as PAH.

The purpose of the present study was to assess the in vivo effects of anti-Gremlin-1 on development of PAH in the chronic hypoxia/SU5416 mouse model.

### Materials and Methods

### Reagents

Imatinib: Science Warehouse 1625-1000.
SU5416: R & D Systems 3037.
αSMA antibody: Dako M085129-2.
VWF antibody: Dako A008202-2.
Biotinylated Goat Anti-Rabbit IgG Antibody: Vector Labs BA-1000.
Biotinylated Horse Anti-Mouse IgG Antibody, rat adsorbed: Vector Labs BA-2001.
Carboxymethyl cellulose: Sigma C5678 419273.
TWEEN 80: Sigma P1754.
Benzyl alcohol: Sigma 305197; 402834.
Sodium chloride: Sigma S7653; VWR 10241.
VECTASTAIN ABC-AP Kit: Vector Labs AK-5000.
VECTASTAIN Elite ABC Kit: Vector Labs PK-6100.
Normal Horse Serum: Vector Labs, catalogue reference S-2000.
polysorbate: Sigma 59924.

### Experimental Protocols

### Animals

C57Bl/6 mice were housed in a specific pathogen free facility, and had access to food and water ad libitum and were exposed to a 12 hour light/dark cycle. This animal study was licensed under the UK Home Office Animals (Scientific Procedures) Act 1986.

### HYPOXIA/SU5416 Mouse Model of Pulmonary Arterial Hypertension

8-10 week old C57Bl/6 female mice were allocated to the groups (Table 6). All groups were weighed and administered subcutaneously (s.c.) with 20 mg/kg SU5416 in 100p of vehicle (0.5% carboxymethyl cellulose (CMC); 0.9% sodium chloride (NaCl); 0.4% polysorbate 80; 0.9% benzyl alcohol in deionized water), as described in Ciuclan et al AJRCCM 2013. As appropriate, a second s.c. injection was administered, as outlined in Table 6, containing either: PBS, 30 mg/kg IgG1, or 30 mg/kg anti-Gremlin-1. Whilst the mice in the Hypoxia+Imatinib group were given chow infused with Imatinib to deliver 100 mg/kg/day. Hypoxia mouse groups were then placed into a normobaric hypoxia (10% O₂) chamber, whilst the mice in the normoxia groups were housed in normoxic (21% O₂)

conditions in the same room as the chamber.

TABLE 6
Treatment groups: C57B1/6 mice were allocated
to the treatment groups as indicated.

| Group | Number of mice |
|---|---|
| Normoxia + PBS | 2 |
| Normoxia + IgG1 | 6 |
| Normoxia + anti-GREMLIN1 | 8 |
| Hypoxia + PBS | 2 |
| Hypoxia + IgG | 6 |
| Hypoxia + anti-GREMLIN1 | 8 |
| Hypoxia + Imatinib | 8 |

On days 7 and 14 all mice were weighed, and received a further s.c. dose of 20 mg/kg SU5416. As appropriate, mice were administered with a further s.c. injection of PBS, 30 mg/kg IgG1, or 30 mg/kg anti-Gremlin-1, (as outlined in table 6). The anti-Gremlin-1 antibody used in these studies was Ab7326 mouse full-length IgG1 format, variant 1, as described in Example 5. On day 21 right ventricular systolic pressures (RVSP) and mean arterial blood pressure (MABP) were obtained, and tissues collected.

### Right Ventricular Systolic Pressures and Tissue Collection

Hemodynamic measurements of RVSP and MABP were obtained from the animals after three weeks of hypoxia exposure and relevant drug treatment as outlined in table 6. The animals were anaesthetised with 1.5% isofluorane and placed supine onto a heating blanket that was thermostatically controlled at 37° C. First, the right jugular vein was isolated and a pressure catheter (Millar mouse SPR-671NR pressure catheter with a diameter of 1.4F, Millar Instruments, UK) introduced and advanced into the right ventricle to determine RVSP. Second, MABP was measured by isolating the left common carotid artery and a pressure catheter introduced. Both RVSP and MABP were recorded onto a precalibrated PowerLab system (ADInstruments, Australia).

Animals were euthanised by via isofluorane anaesthetic overdose and whole blood collected. The whole blood was centrifuged (220×g; 2 min), and serum removed and stored at −80° C. The heart was removed and right and left ventricle weights recorded. Lungs were perfused with 2.5 ml of saline via the right ventricle. The left lung was fixed by inflation with 10% formalin before paraffin embedding and sectioning. The right lung was snap frozen in liquid nitrogen and stored at −80° C.

### Histology

Slides were dewaxed and re-hydrated using xylene and a concentration gradient of ethanol. Slides were immersed in 0.3% H₂O₂ in methanol for 30 minutes to retrieve antigens, washed 3 times in PBS and blocked for 1 hour in 1:30 normal horse serum in PBS. Anti-αSMA primary antibody at a concentration of 1:100 was added to each slide and incubated at 4° C. overnight, then rinsed in PBS for 5 minutes, three times. Biotinylated Horse Anti-Mouse IgG antibody, rat adsorbed secondary antibody was diluted 1:200 and pipetted onto each slide and incubated for 45 minutes; then washed in PBS 3 times for 5 minutes. As per manufacturer's instructions, avidin biotin complex alkaline phosphotase (ABC-AP) was prepared 30 minutes in advance and placed on each slide for 30 minutes; then washed 3 times for 5 minutes. AP substrate was prepared as per kit instructions and pipetted onto each slide and allowed to develop; then washed 3 times for 5 minutes. Anti-vWF primary antibody at a concentration of 1:100 was added to each slide and incubated at 4° C. overnight, then rinsed in PBS for 5 minutes, three times. Biotinylated Goat Anti-Rabbit IgG secondary antibody was diluted 1:200 and left on each slide for 45 minutes; then washed in PBS 3 times for 5 minutes. As per kit instructions ABC was prepared 30 mins in advance and put onto each slide for 30 mins; then washed 3 times for 5 mins. DAB substrate was prepared as per kit instructions and pipetted onto each slide and allowed to develop ˜5-10 mins; then washed 3 times for 5 mins. Slides were counterstained with haematoxylin ˜40 secs, dehydrated and mounted with a coverslip using pertex. All slides were digitally scanned with Hamamatsu NanoZoomer 2.0-HT Slide Scanner (Hamamatsu, Welwyn Garden City, UK).

### Data and Statistical Analysis

Greater than 40 vessels taken at random from each hypoxia group were assessed for the extent of muscularisation. Vessels were scored by at least four independent observers: 0=non-muscularised; 1=partially muscularised; 2=fully muscularised; and the modal value for each vessel determined. The percentage of vessels fully, partially or non muscularised was determined and mean±SEM plotted. One way ANOVA was performed to determine significance *P<0.05; **P<0.01; ***P<0.005; ****P<0.001.

### Results

Administration of SU5416 (20 mg/kg) following exposure to chronic normobaric hypoxia (10% O₂) led to a significant (P<0.01) increase in RVSP compared to normoxia/SU5416 alone for 21 days in both PBS alone or IgG1 control groups (FIG. 12). No significant difference was observed between IgG1 and PBS treatment groups. Effect of drug treatments on raised RVSP in mice administered SU5416 maintained in hypoxia: Imatinib led to a significant (P<0.001) reduction in RVSP compared to control groups. Anti-Gremlin 1 led to a significant (P<0.005) reduction in RVSP compared to IgG1 and PBS control groups. No significant effect of anti-Gremlin 1 was observed on RVSP in animals maintained in normoxia (FIG. 12).

The effect on MABP of anti-Gremlin 1 (n=4) or IgG1 vehicle control (n=4), under hypoxia and normoxia was assessed (FIG. 13). No significant effects of treatments on MABP were observed.

Right heart hypertrophy (right ventricle/left ventricle+septum weights) were determined (FIG. 14). Administration of SU5416 (20 mg/kg) following exposure to chronic normobaric hypoxia (10% O₂) led to a significant (P<0.01) increase in right heart hypertrophy (RV/LV+S) compared to normoxia/SU5416 alone after 21 days in both PBS alone or IgG1 control groups (FIG. 14). No significant effect of drug treatments (imatinib or anti-Gremlin 1;) on raised RVSP in mice administered SU5416 maintained in hypoxia was observed.

The extent of vascular remodelling was assessed by staining paraffin embedded lung sections. Blood vessels were stained for Von Willebrand factor (vWF) to identify endothelial cells and smooth muscle actin (αSMA) to assess the extent of muscularisation. Images were digitised by Hamamatsu NanoZoomer 2.0-HT Slide Scanner and at least 40 vessels taken at random from the normoxia IgG1 and each hypoxia group were assessed for the extent of muscularisation. Vessels were scored by at least four independent observers: 0=non-muscularised; 1=partially muscularised; 2=fully muscularised; and the modal value for each vessel determined and the percentage of non, partial and fully muscularised vessels plotted (FIG. 15). Administration of SU5416 (20 mg/kg) following exposure to chronic normobaric hypoxia (10% O₂) led to a significant increase in full muscularised (P<0.001) and partially muscularised (P<0.01), with a composite significant reduction in none muscularised (P<0.001) vessels. Effect of drug treatments on raised RVSP in mice administered SU5416 maintained in hypoxia: Imatinib led to a significant (P<0.001) reduction in fully muscularised (P<0.01) vessels. Furthermore anti-Gremlin 1 led to a significant (P<0.001 and P<0.05 respectively) compared to hypoxia SU5416 IgG1 control group (FIG. 15). No significant effect of drug treatment (Imatinib or anti-Gremlin 1) on the percentage of partially muscularised vessels compared to the hypoxia SU5416 IgG1 control group was observed. Drug treatment (Imatinib or anti-Gremlin 1) led to an increase in the percentage of non-muscularised vessels compared to the hypoxia SU5416 IgG1 control group, however this failed to reach significance.

### Discussion

Here we assessed the effects of an anti-gremlin-1 antibody on development of PAH in the chronic hypoxia/SU5416 model. The anti-gremlin-1 antibody significantly inhibited RVSPs (FIG. 12) and vascular remodelling (FIG. 15), whilst exhibiting no effect on systemic (MABP) pressures (FIG. 13) in the hypoxia/SU5416 mouse model of PAH. We observed no significant effect by the anti-gremlin-1 antibody on RVSP in normoxia/SU5416 treated mice. The effects of the anti-gremlin1 antibody are consistent with the observations made by Ciuclan and colleagues (AJP 2013) in which they demonstrated antagonism with an anti-gremlin-1 antibody ameliorates raised RVSP and vascular remodelling in the chronic hypoxia/SU5416-mouse model of PAH (FIGS. 12 and 15). In contrast to Ciuclan and colleagues we noted no significant effect on right heart hypertrophy by the anti-gremlin-1 antibody in this study (FIG. 14). However in this study we noted no significant effect by the control drug imatinib on the development of right heart hypertrophy in hypoxia/SU5416 mice. Collectively this study supports the role of gremlin-1 in the development of pulmonary vascular remodelling and raised RVSPs in the chronic hypoxia/SU5416 model of PAH and the use of anti-gremlin-1 antibodies in its treatment.

### REFERENCES

Badesch D B, Champion H C, Sanchez M A, Hoeper M M, Loyd J E, Manes A, McGoon M, Naeije R, Olschewski H, Oudiz R J, Torbicki A. Diagnosis and assessment of pulmonary arterial hypertension. J Am Coll Cardiol. 2009 Jun. 30; 54(1 Suppl):S55-66. doi: 10.1016/j.jacc.2009.04.011. Review. PMID:19555859.
Budd D C, Holmes A M. Targeting TGFβ superfamily ligand accessory proteins as novel therapeutics for chronic lung disorders. Pharmacol Ther. 2012 September; 135(3):279-91. doi: 10.1016/j.pharmthera.2012.06.001. Epub 2012 Jun. 18.
Cahill E, Costello C M, Rowan S C, Harkin S, Howell K, Leonard M O, Southwood M, Cummins E P, Fitzpatrick S F, Taylor C T, Morrell N W, Martin F, McLoughlin P. Gremlin plays a key role in the pathogenesis of pulmonary hypertension. Circulation. 2012 Feb. 21; 125(7):920-30. doi: 10.1161/CIRCULATIONAHA.111.038125. PMID:22247494.
Ciuclan L, Sheppard K, Dong L, Sutton D, Duggan N, Hussey M, Simmons J, Morrell N W, Jarai G, Edwards M, Dubois G, Thomas M, Van Heeke G, England K. Treatment with anti-gremlin 1 antibody ameliorates chronic hypoxia/SU5416-induced pulmonary arterial hypertension in mice. Am J Pathol. 2013 November; 183(5):1461-73. doi: 10.1016/j.ajpath.2013.07.017. PMID:24160323.
Ciuclan L, Hussey M J, Burton V, Good R, Duggan N, Beach S, Jones P, Fox R, Clay I, Bonneau O, Konstantinova I, Pearce A, Rowlands D J, Jarai G, Westwick J, MacLean M R, Thomas M. Imatinib attenuates hypoxia-induced pulmonary arterial hypertension pathology via reduction in 5-hydroxytryptamine through inhibition of tryptophan hydroxylase 1 expression. Am J Respir Crit Care Med. 2013 Jan. 1; 187(1):78-89. doi: 10.1164/rccm.201206-1028OC. PMID:23087024.
Farber H W, Loscalzo J. Pulmonary arterial hypertension. N Engl J Med. 2004 Oct. 14; 351(16):1655-65. Review. PMID:15483284.
Gilbane A J, Derrett-Smith E, Trinder S L, Good R B, Pearce A, Denton C P, Holmes A M. Impaired bone morphogenetic protein receptor II signaling in a transforming growth factor-β-dependent mouse model of pulmonary hypertension and in systemic sclerosis. Am J Respir Crit Care Med. 2015 Mar. 15; 191(6):665-77. doi: 10.1164/rccm.201408-1464OC.
Simonneau G, Robbins I M, Beghetti M, Channick R N, Delcroix M, Denton C P, Elliott C G, Gaine S P, Gladwin M T, Jing Z C, Krowka M J, Langleben D, Nakanishi N, Souza R. Updated clinical classification of pulmonary hypertension. J Am Coll Cardiol. 2009 Jun. 30; 54(1 Suppl):S43-54. doi: 10.1016/j.jacc.2009.04.012. Review. PMID:19555858.
Thomas M, Docx C, Holmes A M, Beach S, Duggan N, England K, Leblanc C, Lebret C, Schindler F, Raza F, Walker C, Crosby A, Davies R J, Morrell N W, Budd D C. Activin-like kinase 5 (ALK5) mediates abnormal proliferation of vascular smooth muscle cells from patients with familial pulmonary arterial hypertension and is involved in the progression of experimental pulmonary arterial hypertension induced by monocrotaline. Am J Pathol. 2009 February; 174(2):380-9. doi: 10.2353/ajpath.2009.080565. Epub 2008 Dec. 30.

Sequence listing
(Human Gremlin-1; Uniprot ID: O60565)
SEQ ID NO: 1
MSRTAYTVGALLLLLGTLLPAAEGKKKGSQGAIPPPDKAQHNDSEQTQSPQQPGSRNRGR
GQGRGTAMPGEEVLESSQEALHVTERKYLKRDWCKTQPLKQTIHEEGCNSRTIINRFCYG
QCNSFYIPRHIRKEEGSFQSCSFCKPKKFTTMMVTLNCPELQPPTKKKRVTRVKQCRCIS
IDLD
(Human truncated Gremlin-1 used in crystallography with
N-terminal tag)
SEQ ID NO: 2
MGSSHHHHHHSSGENLYFQGSAMPGEEVLESSQEALHVTERKYLKRDWCKTQPLKQTIHE
EGCNSRTIINRFCYGQCNSFYIPRHIRKEEGSFQSCSFCKPKKFTTMMVTLNCPELQPPT
KKKRVTRVKQCRCISIDLD
(Ab 7326 HCDR1 combined Kabat & Chothia)
SEQ ID NO: 3
GYTFTDYYMH
(Ab 7326 HCDR1 Kabat)
SEQ ID NO: 4
DYYMH
(Ab 7326 HCDR2 Kabat)
SEQ ID NO: 5
LVDPEDGETIYAEKFQG
(Ab 7326 HCDR3 Kabat)
SEQ ID NO: 6
DARGSGSYYPNHFDY
(Ab 7326 LCDR1 Kabat)
SEQ ID NO: 7
KSSQSVLYSSNNKNYLA
(Ab 7326 LCDR2 Kabat)
SEQ ID NO: 8
WASTRES
(Ab 7326 LCDR3 Kabat)
SEQ ID NO: 9
QQYYDTPT
(Ab 7326 Heavy chain variable region variant 1)
SEQ ID NO: 10
QVQLVESGAEVKKPGATVKISCKVSGYTFTDYYMHWVQQAPGKGLEWMGLVDPEDGETIY
AEKFQGRVTITADTSTDTAYMELSSLRSEDTAVYYCATDARGSGSYYPNHFDYWGQGTLV
TVSS
(Ab 7326 Light chain variable region variant 1)
SEQ ID NO: 11
DIVMTQSPDSLAVSLGERATINCKSSQSVLYSSNNKNYLAWYQQKPGQPPKLLIYWASTR
ESGVPDRFSGSGSGTDFTLTINSLQAEDVAVYFCQQYYDTPTFGQGTRLEIK
(Ab 7326 Heavy chain variable region variant 2)
SEQ ID NO: 12
QVQLVQSGAEVKKPGATVKISCKVSGYTFTDYYMHWVQQAPGKGLEWMGLVDPEDGETIY
AEKFQGRVTITADTSTDTAYMELSSLRSEDTAVYYCATDARGSGSYYPNHFDYWGQGTLV
TVSS
(Ab 7326 Light chain variable region variant 2)
SEQ ID NO: 13
DIVMTQTPDSLAVSLGERATINCKSSQSVLYSSNNKNYLAWYQQKPGQPPKLLIYWASTR
ESGVPDRFSGSGSGTDFTLTINSLQAEDVAVYFCQQYYDTPTFGQGTRLEIK
(Mouse full length IgG1 heavy chain variant 1)
SEQ ID NO: 14
QVQLVESGAEVKKPGATVKISCKVSGYTFTDYYMHWVQQAPGKGLEWMGLVDPEDGETIY
AEKFQGRVTITADTSTDTAYMELSSLRSEDTAVYYCATDARGSGSYYPNHFDYWGQGTLV
TVSSAKTTPPSVYPLAPGSAAQTNSMVTLGCLVKGYFPEPVTVTWNSGSLSSGVHTFPAV
LQSDLYTISSSVTVPSSTWPSETVTCNVAHPASSTKVDKKIVPRDCGCKPCICTVPEVSS
VFIFPPKPKDVLTITLTPKVTCVVVDISKDDPEVQFSWFVDDVEVHTAQTQPREEQFNST
FRSVSELPIMHQDWLNGKEFKCRVNSAAFPAPIEKTISKTKGRPKAPQVYTIPPPKEQMA
KDKVSLTCMITDFFPEDITVEWQWNGQPAENYKNTQPIMDTDGSYFVYSKLNVQKSNWEA
GNTFTCSVLHEGLHNHHTEKSLSHSPGK
(Mouse full length IgG1 light chain variant 1)
SEQ ID NO: 15
DIVMTQSPDSLAVSIGERATINCKSSQSVLYSSNNKNYLAWYQQKPGQPPKLLIYWASTR
ESGVPDRFSGSGSGTDFTLTINSLQAEDVAVYFCQQYYDTPTFGQGTRLEIKRTDAAPTV
SIFPPSSEQLTSGGASVVCFLNNFYPKDINVKWKIDGSERQNGVLNSWTDQDSKDSTYSM
SSTLILIKDEYERHNSYTCEATHKTSTSPIVKSFNRNEC
(Human full length IgG1 heavy chain variant 2)
SEQ ID NO: 16
QVQLVQSGAEVKKPGATVKISCKVSGYTFTDYYMHWVQQAPGKGLEWMGLVDPEDGETIY
AEKFQGRVTITADTSTDTAYMELSSLRSEDTAVYYCATDARGSGSYYPNHFDYWGQGTLV
TVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPE
LLGGPSVFLFPPKPKDTIMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLYSKLTVD
KSRWQQGNVFSCSVMHEALHNHYTQKSLSLSPGK
(Human full length IgG1 light chain variant 2)
SEQ ID NO: 17
DIVMTQTPDSLAVSLGERATINCKSSQSVLYSSNNKNYLAWYQQKPGQPPKLLIYWASTR
ESGVPDRESGSGSGTDFTLTINSLQAEDVAVYFCQQYYDTPTFGQGTRLEIKRIVAAPSV
FIFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
(Fab heavy chain variant 1)
SEQ ID NO: 18
QVQLVESGAEVKKPGATVKISCKVSGYTFTDYYMHWVQQAPGKGLEWMGLVDPEDGETIY
AEKFQGRVTITADTSTDTAYMELSSLRSEDTAVYYCATDARGSGSYYPNHFDYWGQGTLV
TVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSC
(Fab light chain variant 1)
SEQ ID NO: 19
DIVMTQSPDSLAVSLGERATINCKSSQSVLYSSNNKNYLAWYQQKPGQPPKLLIYWASTR
ESGVPDRFSGSGSGTDFTLTINSLQAEDVAVYFCQQYYDTPTFGQGTRLEIKRTVAAPSV
FIFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
(Human truncated Gremlin-1 used in crystallography
without N-terminal tag)
SEQ ID NO: 20
AMPGEEVLESSQEALHVTERKYLKRDWCKTQPLKQTIHEEGCNSRTIINRFCYGQCNSFY
IPRHIRKEEGSFQSCSFCKPKKFTTMMVTLNCPELQPPTKKKRVTRVKQCRCISIDLD
(Mature Gremlin-1 sequence of SEQ ID NO: 1 lacking the
signal peptide of amino acids 1-21)
SEQ ID NO: 21
KKKGSQGAIPPPDKAQHNDSEQTQSPQQPGSRNRGRGQGRGTAMPGEEVLESSQEALHVT
ERKYLKRDWCKTQPLKQTIHEEGCNSRTIINRFCYGQCNSFYIPRHIRKEEGSFQSCSFC
KPKKFTTMMVTLNCPELQPPTKKKRVTRVKQCRCISIDLD
(Human IgG4P heavy chain variant 1)
SEQ ID NO: 22
QVQLVESGAEVKKPGATVKISCKVSGYTFTDYYMHWVQQAPGKGLEWMGLVDPEDGETIY
AEKFQGRVTITADTSTDTAYMELSSLRSEDTAVYYCATDARGSGSYYPNHFDYWGQGTLV
TVSSASTKGPSVFPLAPCSRSTSESTAALGCLVKDYFPEPVTVSWNSGALTSGVHTFPAV
LQSSGLYSLSSVVTVPSSSLGTKTYTCNVDHKPSNTKVDKRVESKYGPPCPPCPAPEFLG
GPSVFLFPPKPKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREPQVYTLPPSQE
EMTKNQVSLTCLVKGFYPSDIAVEWESNGQPENNYKTTPPVLDSDGSFFLYSRLTVDKSR
WQEGNVFSCSVMHEALHNHYTQKSLSLSLGK
(Human IgG4P light chain variant 1)
SEQ ID NO: 23
DIVMTQSPDSLAVSLGERATINCKSSQSVLYSSNNKNYLAWYQQKPGQPPKLLIYWASTR
ESGVPDRFSGSGSGTDFTLTINSLQAEDVAVYFCQQYYDTPTFGQGTRLEIKRTVAAPSV
FIFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
(Human IgG1 heavy chain DNA variant 1)
SEQ ID NO: 24
caagtgcaactggtggaatccggggccgaagtgaaaaagcccggagccactgtgaagatc
tcttgcaaagtgtccggctacaccttcaccgactattacatgcactgggtccagcaggca
cctgggaagggccttgagtggatgggtctggtcgatcccgaggacggcgaaactatctac
gccgagaagttccagggtcgcgtcaccatcaccgccgacacttccaccgacaccgcgtac
atggagctgtccagcttgaggtccgaggacacagccgtgtactactgcgccacggatgct
cggggaagcggcagctactacccgaaccacttcgactactggggacagggcactctcgtg
actgtctcgagcgcttctacaaagggcccctccgtgttcccgctcgctccatcatcgaag
tctaccagcggaggcactgcggctctcggttgcctcgtgaaggactacttcccggagccg
gtgaccgtgtcgtggaacagcggagccctgaccagcggggtgcacacctttccggccgtc
ttgcagtcaagcggcctttactccctgtcatcagtggtgactgtcccgtccagctcattg
ggaacccaaacctacatctgcaatgtgaatcacaaacctagcaacaccaaggttgacaag
aaagtcgagcccaaatcgtgtgacaagactcacacttgtccgccgtgcccggcacccgaa
ctgctgggaggtcccagcgtctttctgttccctccaaagccgaaagacacgctgatgatc
tcccgcaccccggaggtcacttgcgtggtcgtggacgtgtcacatgaggacccagaggtg
aagttcaattggtacgtggatggcgtcgaagtccacaatgccaaaactaagcccagagaa
gaacagtacaattcgacctaccgcgtcgtgtccgtgctcacggtgttgcatcaggattgg
ctgaacgggaaggaatacaagtgcaaagtgtccaacaaggcgctgccggcaccgatcgag
aaaactatctccaaagcgaagggacagcctagggaacctcaagtctacacgctgccacca
tcacgggatgaactgactaagaatcaagtctcactgacttgtctggtgaaggggttttac
cctagcgacattgccgtggagtgggaatccaacggccagccagagaacaactacaagact
acccctccagtgctcgactcggatggatcgttcttcctttactcgaagctcaccgtggat
aagtcccggtggcagcagggaaacgtgttctcctgctcggtgatgcatgaagccctccat
aaccactatacccaaaagtcgctgtccctgtcgccgggaaag
(Human IgG1 light chain DNA variant 1)
SEQ ID NO: 25
gacattgtgatgacccagtcccccgattcgcttgcggtgtccctgggagaacgggccacc
attaactgcaagagctcacagtccgtcctgtattcatcgaacaacaagaattacctcgca
tggtatcagcagaagcctggacagcctcccaagctgctcatctactgggctagcacccgc
gaatccggggtgccggatagattctccggatcgggttcgggcactgacttcactctgact
atcaactcactgcaagccgaggatgtcgcggtgtacttctgtcagcagtactacgacacc
ccgacctttggacaaggcaccagactggagattaagcgtacggtggccgctccctccgtg
ttcatcttcccaccctccgacgagcagctgaagtccggcaccgcctccgtcgtgtgcctg
ctgaacaacttctacccccgcgaggccaaggtgcagtggaaggtggacaacgccctgcag
tccggcaactcccaggaatccgtcaccgagcaggactccaaggacagcacctactccctg
tcctccaccctgaccctgtccaaggccgactacgagaagcacaaggtgtacgcctgcgaa
gtgacccaccagggcctgtccagccccgtgaccaagtccttcaaccggggcgagtgc
(Human IgG4P heavy chain DNA variant 1)
SEQ ID NO: 26
caagtgcaactggtggaatccggggccgaagtgaaaaagcccggagccactgtgaagatc
tcttgcaaagtgtccggctacaccttcaccgactattacatgcactgggtccagcaggca
cctgggaagggccttgagtggatgggtctggtcgatcccgaggacggcgaaactatctac
gccgagaagttccagggtcgcgtcaccatcaccgccgacacttccaccgacaccgcgtac
atggagctgtccagcttgaggtccgaggacacagccgtgtactactgcgccacggatgct
cggggaagcggcagctactacccgaaccacttcgactactggggacagggcactctcgtg
actgtctcgagcgcttctacaaagggcccctccgtgttccctctggccccttgctcccgg
tccacctccgagtctaccgccgctctgggctgcctggtcaaggactacttccccgagccc
gtgacagtgtcctggaactctggcgccctgacctccggcgtgcacaccttccctgccgtg
ctgcagtcctccggcctgtactccctgtcctccgtcgtgaccgtgccctcctccagcctg
ggcaccaagacctacacctgtaacgtggaccacaagccctccaacaccaaggtggacaag
cgggtggaatctaagtacggccctccctgccccccctgccctgcccctgaatttctgggc
ggaccttccgtgttcctgttccccccaaagcccaaggacaccctgatgatctcccggacc
cccgaagtgacctgcgtggtggtggacgtgtcccaggaagatcccgaggtccagttcaat
tggtacgtggacggcgtggaagtgcacaatgccaagaccaagcccagagaggaacagttc
aactccacctaccgggtggtgtccgtgctgaccgtgctgcaccaggactggctgaacggc
aaagagtacaagtgcaaggtgtccaacaagggcctgccctccagcatcgaaaagaccatc
tccaaggccaagggccagccccgcgagccccaggtgtacaccctgccccctagccaggaa
gagatgaccaagaaccaggtgtccctgacctgtctggtcaagggcttctacccctccgac
attgccgtggaatgggagtccaacggccagcccgagaacaactacaagaccaccccccct
gtgctggacagcgacggctccttcttcctgtactctcggctgaccgtggacaagtcccgg
tggcaggaaggcaacgtcttctcctgctccgtgatgcacgaggccctgcacaaccactac
acccagaagtccctgtccctgagcctgggcaag
(Human IgG4P light chain DNA variant 1)
SEQ ID NO: 27
gacattgtgatgacccagtcccccgattcgcttgcggtgtccctgggagaacgggccacc
attaactgcaagagctcacagtccgtcctgtattcatcgaacaacaagaattacctcgca
tggtatcagcagaagcctggacagcctcccaagctgctcatctactgggctagcacccgc
gaatccggggtgccggatagattctccggatcgggttcgggcactgacttcactctgact
atcaactcactgcaagccgaggatgtcgcggtgtacttctgtcagcagtactacgacacc
ccgacctttggacaaggcaccagactggagattaagcgtacggtggccgctccctccgtg
ttcatcttcccaccctccgacgagcagctgaagtccggcaccgcctccgtcgtgtgcctg
ctgaacaacttctacccccgcgaggccaaggtgcagtggaaggtggacaacgccctgcag
tccggcaactcccaggaatccgtcaccgagcaggactccaaggacagcacctactccctg
tcctccaccctgaccctgtccaaggccgactacgagaagcacaaggtgtacgcctgcgaa
gtgacccaccagggcctgtccagccccgtgaccaagtccttcaaccggggcgagtgc
(Mouse full length IgG1 heavy chain variant 2)
SEQ ID NO: 28
QVQLVQSGAEVKKPGATVKISCKVSGYTFTDYYMHWVQQAPGKGLEWMGLVDPEDGETIY
AEKFQGRVTITADTSTDTAYMELSSLRSEDTAVYYCATDARGSGSYYPNHFDYWGQGTLV
TVSSAKTTPPSVYPLAPGSAAQTNSMVTLGCLVKGYFPEPVTVTWNSGSLSSGVHTFPAV
LQSDLYTLSSSVTVPSSTWPSETVTCNVAHPASSTKVDKKIVPRDCGCKPCICTVPEVSS
VFIFPPKPKDVLTITLTPKVTCVVVDISKDDPEVQFSWFVDDVEVHTAQTQPREEQFNST
FRSVSELPIMHQDWLNGKEFKCRVNSAAFPAPIEKTISKTKGRPKAPQVYTIPPPKEQMA
KDKVSLTCMITDFFPEDITVEWQWNGQPAENYKNTQPIMDTDGSYFVYSKLNVOKSNWEA
GNTFTCSVLHEGLHNHHTEKSLSHSPGK
(Mouse full length IgG1 light chain variant 2)
SEQ ID NO: 29
DIVMTQTPDSLAVSLGERATINCKSSQSVLYSSNNKNYLAWYQQKPGQPPKLLIYWASTR
ESGVPDRFSGSGSGTDFTLTINSLQAEDVAVYFCQQYYDTPTFGQGTRLEIKRTDAAPTV
SIFPPSSEQLTSGGASVVCFINNFYPKDINVKWKIDGSERQNGVLNSWTDQDSKDSTYSM
SSTLTLTKDEYERHNSYTCEATHKTSTSPIVKSFNRNEC
(Human full length IgG1 heavy chain variant 1)
SEQ ID NO: 30
QVQLVESGAEVKKPGATVKISCKVSGYTFTDYYMHWVQQAPGKGLEWMGLVDPEDGETIY
AEKFQGRVTITADTSTDTAYMELSSLRSEDTAVYYCATDARGSGSYYPNHFDYWGQGTLV
TVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLYSKLTVD
KSRWQQGNVFSCSVMHEALHNHYTQKSLSLSPGK
(Human full length IgG1 light chain variant 1)
SEQ ID NO: 31
DIVMTQSPDSLAVSLGERATINCKSSQSVLYSSNNKNYLAWYQQKPGQPPKLLIYWASTR
ESGVPDRFSGSGSGTDFTLTINSLQAEDVAVYFCQQYYDTPTFGQGTRLEIKRTVAAPSV
FIFPPSDEQLKSGTASVVCLLNNFYPREAKVQWKVDNALQSGNSQESVTEQDSKDSTYSE
SSTLTLSKADYEKHKVYACEVTHQGLSSPVTKSENRGEC
(Fab heavy chain variant 2)
SEQ ID NO: 32
QVQLVQSGAEVKKPGATVKISCKVSGYTFTDYYMHWVQQAPGKGLEWMGLVDPEDGETIY
AEKFQGRVTITADTSTDTAYMELSSLRSEDTAVYYCATDARGSGSYYPNHFDYWGQGTLV
TVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSC
(Fab light chain variant 2)
SEQ ID NO: 33
DIVMTQTPDSLAVSLGERATINCKSSQSVLYSSNNKNYLAWYQQKPGQPPKLLIYWASTR
ESGVPDRFSGSGSGTDFTLTINSLQAEDVAVYFCQQYYDTPTFGQGTRLEIKRTVAAPSV
FIFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
(Human IgG4P heavy chain variant 2)
SEQ ID NO: 34
QVQLVQSGAEVKKPGATVKISCKVSGYTFTDYYMHWVQQAPGKGLEWMGLVDPEDGETIY
AEKFQGRVTITADTSTDTAYMELSSLRSEDTAVYYCATDARGSGSYYPNHFDYWGQGTLV
TVSSASTKGPSVFPLAPCSRSTSESTAALGCLVKDYFPEPVTVSWNSGALTSGVHTFPAV
LQSSGLYSLSSVVTVPSSSLGTKTYTCNVDHKPSNTKVDKRVESKYGPPCPPCPAPEFLG
GPSVFLFPPKPKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREPQVYTLPPSQE
EMTKNQVSLTCLVKGFYPSDIAVEWESNGQPENNYKTTPPVLDSDGSFFLYSRLTVDKSR
WQEGNVFSCSVMHEALHNHYTQKSLSLSLGK
(Human IgG4P light chain variant 2)
SEQ ID NO: 35
DIVMTQTPDSLAVSLGERATINCKSSQSVLYSSNNKNYLAWYQQKPGQPPKLLIYWASTR
ESGVPDRFSGSGSGTDFTLTINSLQAEDVAVYFCQQYYDTPTFGQGTRLEIKRTVAAPSV
FIFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC

We claim:

1. An antibody which binds to an epitope on Gremlin-1 comprising at least one residue selected from Ile131, Lys147, Lys148, Phe149, Thr150, Thr151, Arg169, Lys174 and Gln175, wherein the residue numbering is according to SEQ ID NO: 1.

2. The antibody of claim 1, which binds an epitope comprising all of Ile131, Lys147, Lys148, Phe149, Thr150, Thr151, Arg169, Lys174 and Gln175.

3. The antibody of claim 1, wherein Lys147, Lys148, Phe149, Thr150, Thr151, Arg169, Lys174 and Gln175 are located on the same Gremlin-1 monomer and Ile131 is located on the second Gremlin-1 monomer.

4. The antibody of claim 1, wherein an antibody binds a Gremlin-1 residue if the antibody paratope is within 4 Å of the Gremlin-1 residue as determined by x-ray crystallography.

5. The antibody of claim 1, which restores signal in the Hek-Id1 reporter gene assay.

6. A method of treating renal fibrosis, diabetic nephropathy, idiopathic pulmonary fibrosis, pulmonary arterial hypertension, angiogenesis and/or cancer, comprising administering a therapeutically effective amount of a monoclonal anti-Gremlin-1 antibody comprising a HCDR1/HCDR2/HCDR3/LCDR1/LCDR2/LCDR3 sequence combination of SEQ ID NOs: 4/5/6/7/8/9 or SEQ ID NOs: 3/5/6/7/8/9 or a pharmaceutical composition thereof to a patient in need thereof.

7. A crystal of Gremlin-1 or a crystal of human Gremlin-1 in complex with an antibody.

8. The crystal of claim 7, wherein the Gremlin-1 is human Gremlin-1.

9. The crystal of claim 8, consisting of a C2 space group with unit cell dimensions of a=84.55 Å, b=107.22 Å and c=77.09 Å for human Gremlin-1.

10. The crystal of claim 7, wherein the antibody in complex with human Gremlin 1 comprises a heavy chain of SEQ ID NO: 18 and a light chain of SEQ ID NO: 19.

11. A machine readable data storage medium which comprises data storage material encoded with machine readable data defined by the structure coordinates of Gremlin-1 in Table 1 or coordinates defining homologues of the structure.

12. A method of screening for modulatory agents of Gremlin-1 activity, comprising the steps of:
(a) identifying a ligand binding site from the structural coordinates in Table 1;
(b) identifying candidate agents which interact with at least part of the ligand binding site; and
(c) obtaining or synthesising said agent.

13. The method of claim 12, wherein steps (a) and (b) are performed in silico.

14. The method of claim 12, wherein the agent is an inhibitor of Gremlin-1 activity.

15. The method of claim 12, wherein the method comprises identifying candidate agents which interact with at least one of the following residues of Gremlin-1:
Trp93;
Phe117;
Tyr119;
Phe125;
Tyr126; and/or
Phe138,
wherein the residue numbering is based on SEQ ID NO: 1.

16. The method of claim 15, wherein the agent interacts with all the following residues of Gremlin-1: Trp93, Phe117, Tyr119, Phe125, Tyr126 and Phe138.

17. The method of claim 15, wherein a candidate agent interacts with Gremlin-1 if the agent is within 6 Å of a residue as determined by x-ray crystallography.

18. The method of claim 12, wherein the ligand binding site is an allosteric site.

19. The method of claim 18, wherein the method comprises identifying candidate agents which interact with at least one of the following residues of Gremlin-1: Ile131, Lys147, Lys148, Phe149, Thr150, Thr151, Arg169, Lys174 and Gln175, wherein the residue numbering is based on SEQ ID NO: 1.

20. The method of claim 19, wherein the agent interacts with all the following residues of Gremlin-1: Ile131, Lys147, Lys148, Phe149, Thr150, Thr151, Arg169, Lys174 and Gln175.

21. The method of claim 19, wherein Lys147, Lys148, Phe149, Thr150, Thr151, Arg169, Lys174 and Gln175 are located on the same Gremlin-1 monomer and Ile131 is located on the second Gremlin-1 monomer.

22. The method of claim 18, wherein a candidate agent interacts with Gremlin-1 if the agent is within 4 Å of a residue as determined by x-ray crystallography.

23. The method of claim 14, wherein the agent is a small molecule or antibody.

24. A Gremlin-1 modulating agent identified by the method of claim 12.
